(19) United States
(12) Patent Application Publication
(10) Pub. No.: US 2010/0210680 A1
(43) Pub. Date: Aug. 19, 2010

(54) TRICYCLIC HETEROCYCLIC DERIVATIVES

(75) Inventors: Simon James Anthony Grove, Newhouse (GB); Takao Kiyoi, Newhouse (GB); Ashvinkumar Dhirubhai Mistry, Newhouse (GB); Peter Christopher Ray, Newhouse (GB); Grant Wishart, Newhouse (GB)

Correspondence Address: ORGANON USA, INC.;c/o MERCK, 2000 Galloping Hill Road, Mail Stop: K-6-1, 1990, Kenilworth, NJ, 07033, US

(73) Assignee: N.V. ORGANON

(21) Appl. No.: 12/678,184

(22) PCT Filed: Sep. 15, 2008
(86) PCT No.: PCT/EP08/62229

§ 371 Date: Apr. 22, 2010

(30) Foreign Application Priority Data
Sep. 17, 2007 (EP) 07116561.7

(51) Int. Cl. A61K 31/436; C07D 491/052; A61K 31/407; A61K 31/422; A61P 3/04; A61P 25/18

(52) U.S. Cl. 514/291; 548/430; 514/411; 546/89; 548/247; 514/378

(57) ABSTRACT

The present invention relates to a tricyclic heterocyclic derivative of Formula (I) wherein the variables are as defined in the specification. The present invention further relates to pharmaceutical compositions comprising these compounds and to their use in therapy, in particular for the treatment of serotonin-mediated disorders such as obesity, schizophrenia and cognitive dysfunction.

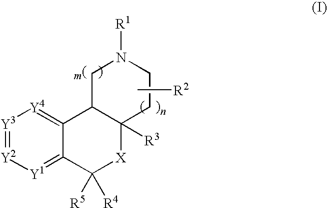

The present invention relates to tricyclic heterocyclic derivatives, to pharmaceutical compositions comprising these compounds and to their use in therapy, in particular for the treatment of serotonin-mediated disorders such as obesity, schizophrenia and cognitive dysfunction.

The 5-hydroxytryptamine-2 (5-HT₂) receptors are a family of G-protein coupled receptors comprising three members (5-HT_{2A}, 5-HT_{2B} and 5-HT_{2C}). 5-HT₂ subtypes activate the phospholipase C second messenger pathway, resulting in phosphoinositide hydrolysis and a transient increase in intracellular calcium. Certain 5-HT₂ subtypes can also activate the phospholipase A2 pathway, leading to release of arachidonic acid. The human 5-HT_{2C} receptor was cloned in 1991 and unlike the 5-HT_{2A} and 5-HT_{2B} receptors, its expression appears to be restricted to the central nervous system (CNS). The 5-HT_{2C} receptor subtype has been implicated in a wide variety of conditions including obesity, anxiety, depression, obsessive compulsive disorder, schizophrenia, migraine and erectile dysfunction. Recently, novel 5-HT_{2C} selective compounds such as WAY-163909 (Dunlop J, CNS Drug Reviews 2006, 12(3), 167-177), CP-809, 101 (Siuciak J. A, Neuropharmacology 2007, 52, 279-290) and (R)-9-ethyl-1,3,4,10b-tetrahydro-7-trifluoromethylpyrazino[2,1-a]isoindol-6(2H)-one (Wacker D. A et al, J. Med. Chem. 2007, 50(6), 1365-1379) have been reported to have robust dose-dependent positive effects on animal models of obesity, schizophrenia and cognitive dysfunction. In spite of the availability of these compounds, however, there remains a need for further 5-HT_{2c} receptor modulators which are safe and effective.

Benzo[4,5]pyrano[2,3-c]pyrrole derivatives have been disclosed in EP-A-0050387 and Loozen et al, Journal of the Royal Netherlands Chemical Society, 101/9, 1982 as dopaminergic molecules. U.S. Pat. No. 4,132,709 and U.S. Pat. No. 4,132,710 relate to certain hexahydro-benzopyranopyridine derivatives indicated to be useful as diuretic, anorexic, antidepressant, anticonvusant and antihypertensive agents.

In a first aspect, the present invention relates to a tricyclic heterocyclic derivative of Formula I

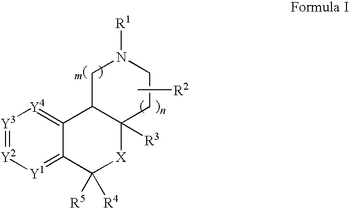

wherein,
m is 1 or 2;
n is 0 or 1;
R¹ is H, C₁₋₆alkyl, C₂₋₆alkenyl, C₂₋₆alkynyl, C₃₋₇cycloalkyl, C₃₋₇cycloalkylC₁₋₂alkyl, C₁₋₄alkyloxyC₂₋₃alkyl or C₆₋₁₀arylC₁₋₂alkyl, said C₁₋₆alkyl, C₂₋₆alkenyl, C₂₋₆alkynyl, C₃₋₇cycloalkyl, C₃₋₇cycloalkylC₁₋₂alkyl, C₁₋₄alkyloxyC₂₋₃alkyl and C₆₋₁₀arylC₁₋₂alkyl being optionally substituted with one or more halogens;
R² is H, C₁₋₆alkyl, C₃₋₇cycloalkyl or C₃₋₇cycloalkylC₁₋₂alkyl, said C₁₋₆alkyl, C₃₋₇cycloalkyl and C₃₋₇cycloalkylC₁₋₂alkyl being optionally substituted with one or more halogens;
R³ is H, C₁₋₆alkyl, C₃₋₇cycloalkyl, C₃₋₇cycloalkylC₁₋₂alkyl or C₁₋₄alkyloxyC₁₋₂alkyl, said C₁₋₆alkyl, C₃₋₇cycloalkyl, C₃₋₇cycloalkylC₁₋₂alkyl and C₁₋₄alkyloxyC₁₋₂alkyl being optionally substituted with one or more halogens;
R⁴ and R⁵ are each independently H, C₁₋₆alkyl, C₃₋₇cycloalkyl, C₃₋₇cycloalkylC₁₋₂alkyl or C₁₋₄alkyloxyC₁₋₂alkyl said C₁₋₆alkyl, C₃₋₇cycloalkyl, C₃₋₇cycloalkylC₁₋₂alkyl and C₁₋₄alkyloxyC₁₋₂alkyl being optionally independently substituted with one or more halogens or R⁴ and R⁵ together with the carbon to which they are bonded form a 3-6 membered carbocyclic ring optionally comprising a further heteroatom selected from O and S;

### X is O, S, SO, SO₂, OCR^{4′}R^{5′} or CR^{4′}R^{5′}O;

R^{4′} and R^{5′} are each independently H, C₁₋₆alkyl, C₃₋₇cycloalkyl or C₃₋₇cycloalkylC₁₋₂alkyl, said C₁₋₆alkyl, C₃₋₇cycloalkyl and C₃₋₇cycloalkylC₁₋₂alkyl being optionally independently substituted with one or more halogens;

### Y¹ is N or CR⁶;

### Y² is N or CR⁷;

### Y³ is N or CR⁸;

Y⁴ is N or CR⁹ with the proviso that no more than one of Y¹-Y⁴ can be N simultaneously;
R⁶, R⁷ and R⁸ are each independently selected from H, C₁₋₆alkyl, C₂₋₆alkenyl, C₂₋₆alkynyl, C₃₋₇cycloalkyl, C₃₋₇cycloalkylC₁₋₂alkyloxy, C₁₋₆alkyloxy, C₁₋₄alkyloxyC₁₋₂alkyl, C₁₋₆alkylSC₁₋₂alkyl, C₁₋₆alkylSO₂C₁₋₂alkyl, SC₁₋₆alkyl, SOC₁₋₆alkyl, SO₂C₁₋₆alkyl, NR¹⁰R¹¹, CO₂R¹², NR¹³SO₂R¹⁴, CONR¹⁵R¹⁶, SO₂NR¹⁷R¹⁸, C₆₋₁₀aryl, C₆₋₁₀arylC₁₋₂alkyloxy, CN, halogen and a 5-6 membered saturated or unsaturated heterocyclic ring system comprising 1-2 heteroatoms independently selected from N, O and S, wherein said C₁₋₆alkyl, C₃₋₇cycloalkyl, C₃₋₇cycloalkylC₁₋₂alkyloxy and C₁₋₆alkyloxy are optionally independently substituted with one or more halogens and wherein said C₆₋₁₀aryl, C₆₋₁₀arylC₁₋₂alkyloxy and 5-6 membered saturated or unsaturated heterocyclic ring system comprising 1-2 heteroatoms independently selected from N, O and S are optionally independently substituted with one or more substituents selected from methyl, halogen and methoxy or R⁶ and R⁷ or R⁷ and R⁸ together with the atoms to which they are bonded form a 5-7 membered unsaturated carbocyclic ring optionally comprising 1-2 heteroatoms selected from N, O and S and optionally substituted with methyl or halogen;
R⁹ is H, C₁₋₆alkyl, C₁₋₆alkyloxy, C₃₋₇cycloalkyl, CN or halogen said C₁₋₆alkyl, C₁₋₆alkyloxy and C₃₋₇cycloalkyl being optionally independently substituted with one or more halogens;
R¹⁰ and R¹¹ are each independently H, C₁₋₆alkyl, C₃₋₇cycloalkyl or COC₁₋₆alkyl said C₁₋₆alkyl being optionally substituted with one or more halogens;
R¹² is C₁₋₆alkyl;
R¹³ is H or C₁₋₆alkyl;
R¹⁴ is C₁₋₆alkyl;
R¹⁵ and R¹⁶ are each independently H or C₁₋₆alkyl and
R¹⁷ and R¹⁸ are each independently H or C₁₋₆alkyl;
with the proviso that when R⁶ and R⁹ are H, R⁷ and R⁸ cannot independently or together be H, hydroxy, methoxy or benzyloxy,
or a pharmaceutically acceptable salt or solvate thereof.

The term C₁₋₆alkyl, as used herein, represents a branched or unbranched alkyl group having 1-6 carbon atoms. Examples of such groups are methyl, ethyl, isopropyl, tertiary butyl and hexyl. Similarly the terms C₁₋₂alkyl, C₁₋₄alkyl and C₂₋₃alkyl, as used herein, represent a branched or unbranched alkyl group having 1-2, 1-4 and 2-3 carbon atoms respectively.

The term C₂₋₆alkenyl, as used herein, represents a branched or unbranched alkenyl group having 2-6 carbon atoms. Examples of such groups are ethenyl and isopropenyl.

The term C₂₋₆alkynyl, as used herein, represents a branched or unbranched alkynyl group having 2-6 carbon atoms. Examples of such groups are ethynyl and propynyl.

The term C₁₋₆alkyloxy, as used herein, represents a branched or unbranched alkyloxy group having 1-6 carbon atoms. Examples of such groups are methoxy, ethoxy, isopropyloxy and tertiary butyloxy. Likewise the terms C₁₋₂alkyloxy and C₁₋₄alkyloxy as used herein, represent a branched or unbranched alkyloxy group having 1-2 and 1-4 carbon atoms respectively.

The term C₁₋₄alkyloxyC₂₋₃alkyl, as used herein, represents a C₂₋₃alkyl group which is substituted with a C₁₋₄alkyloxy group. Examples of such groups are methoxyethyl and ethoxyethyl. Similarly, the term C₁₋₄alkyloxyC₁₋₂alkyl, as used herein, represents a C₁₋₂alkyl group which is substituted with a C₁₋₄alkyloxy group.

The term C₆₋₁₀aryl, as used herein, represents an aromatic group having 6-10 carbon atoms, said aromatic group comprising a single ring or two rings fused together at adjacent carbon atoms. Examples of such groups include phenyl and naphthyl.

The term C₆₋₁₀arylC₁₋₂alkyl, as used herein, represents a C₁₋₂alkyl group which is substituted with a C₆₋₁₀aryl group. Examples of such groups are benzyl and phenylethyl.

The term C₆₋₁₀arylC₁₋₂alkyloxy, as used herein, represents a C₁₋₂alkyloxy group which is substituted with a C₆₋₁₀aryl group. Examples of such groups are benzyloxy and phenylethyloxy.

The term C₃₋₇cycloalkyl, as used herein, represents a branched or unbranched cyclic alkyl group having 3-7 carbon atoms. Examples of such groups are cyclopropyl, cyclopentyl and 2-methylcyclopentyl.

The term C₃₋₇cycloalkylC₁₋₂alkyl, as used herein, represents a C₁₋₂alkyl group which is substituted with a C₃₋₇cycloalkyl group. Examples of such groups are cyclopropylmethyl, and 2-cyclobutylethyl.

The term C₃₋₇cycloalkylC₁₋₂alkyloxy, as used herein, represents a C₁₋₂alkyloxy group which is substituted with a C₃₋₇cycloalkyl group. Examples of such groups are cyclopropylmethyl, and 2-cyclobutylethyl.

The term SC₁₋₆alkyl, as used herein represents a thioalkyl group, for example a SCH₃ or SCH₂CH₃ group. Similarly the term SOC₁₋₆alkyl, as used herein represents an alkylsulphinyl group, for example a SOCH₃ or SOCH₂CH₃ group and the term SO₂C₁₋₆alkyl, as used herein represents an alkylsulphonyl group, for example a SO₂CH₃ or SO₂CH₂CH₃ group.

The term C₁₋₆alkylSC₁₋₂alkyl, as used herein, represents a C₁₋₂alkyl group which is substituted with a SC₁₋₆alkyl group. Examples of such groups are CH₂SCH₃ and CH₂SCH₂CH₃. Similarly term C₁₋₆alkylSO₂C₁₋₂alkyl, as used herein, represents a C₁₋₂alkyl group which is substituted with a SO₂C₁₋₆alkyl group. Examples of such groups are CH₂SO₂CH₃ and CH₂SO₂CH₂CH₃.

The term halogen, as used herein, represents a F, Cl, Br or I atom.

The term solvate, as used herein, refers to a complex of variable stoichiometry formed by a solvent and a solute (in this invention, a compound of Formula I). Such solvents may not interfere with the biological activity of the solute. Examples of suitable solvents include water, methanol, ethanol and acetic acid.

Examples of 5 to 6 membered saturated or unsaturated heterocyclic ring systems comprising 1-2 heteroatoms selected from O, S and N include furan, pyrrole, thiophene, imidazole, pyrazole, thiazole, pyridine, pyrimidine, piperidine, pyrrolidine and tetrahydropyridine.

In one embodiment of the present invention, m is 1. In another embodiment, m is 2.

In a further embodiment of the present invention, n is 0. In another embodiment, n is 1.

In a further embodiment of the present invention, R¹ is H or C₁₋₆alkyl optionally substituted with one or more halogens. In another embodiment, R¹ is H, methyl or ethyl. In another embodiment, R¹ is H.

In a further embodiment of the present invention, R¹ is C₃₋₇cycloalkylC₁₋₂alkyl optionally substituted with one or more halogens. In another embodiment, R¹ is cyclopropylmethyl optionally substituted with one or more halogens.

In a further embodiment of the present invention, R¹ is C₆₋₁₀arylC₁₋₂alkyl, optionally substituted with one or more halogens. In another embodiment, R¹ is benzyl optionally substituted with one or more halogens.

In a further embodiment of the present invention, R² is H or C₁₋₆alkyl optionally substituted with one or more halogens. In another embodiment, R² is H or methyl, optionally substituted with 1-3 halogens. In another embodiment, R² is H.

In a further embodiment of the present invention, R³ is H or C₁₋₆alkyl optionally substituted with one or more halogens. In another embodiment, R³ is H, methyl or ethyl optionally substituted with 1-3 halogens. In another embodiment R³ is H, methyl, fluoromethyl, trifluoromethyl or ethyl.

In a further embodiment of the present invention R⁴, R^{4′}, R⁵ and R^{5′} are each independently H or C₁₋₆alkyl optionally substituted with one or more halogens. In another embodiment R⁴, R^{4′}, R⁵ and R^{5′} are each independently H or methyl, optionally substituted with 1-3 halogens. In another embodiment, R⁴, R^{4′}, R⁵ and R^{5′} are independently H or methyl. In another embodiment, R⁴, R^{4′}, R⁵ and R^{5′} are H.

In a further embodiment of the present invention, X is O;

In a further embodiment of the present invention, X is S, SO or SO₂;

In a further embodiment of the present invention, X is OCR^{4′}R^{5′} or CR^{4′}R^{5′}O, wherein R^{4′} and R^{5′} have the previously defined meanings;

In a further embodiment of the present invention, Y¹ is CR⁶, wherein R⁶ has the previously defined meanings;

In a further embodiment of the present invention, Y² is CR⁷, wherein R⁷ has the previously defined meanings;

In a further embodiment of the present invention, Y³ is CR⁸, wherein R⁸ has the previously defined meanings;

In a further embodiment of the present invention, Y⁴ is CR⁹, wherein R⁹ has the previously defined meanings;

In a further embodiment of the present invention, R⁶ is H, C₁₋₆alkyl, C₂₋₆alkenyl, C₃₋₇cycloalkyl, C₁₋₆alkyloxy, C₆₋₁₀aryl, SC₁₋₆alkyl, NR¹⁰R¹¹ or halogen, said C₁₋₆alkyl and C₁₋₆alkyloxy being optionally substituted with one or more halogens, wherein R¹⁰ and R¹¹ have the previously defined meanings. In another embodiment, R⁶ is H, chloro, bromo, methyl, trifluoromethyl, ethyl, isopropenyl, (Z)-2-propenyl, n-propyl, isopropyl, cyclopropyl, 2-methylpropyl, cyclopentyl, N-methyl-N-ethylamino, N-methyl-N-isopropylamino, methoxy, ethoxy, isopropyloxy, cyclopropyloxy, phenyl, methylthio or N,N-dimethylamino.

In a further embodiment of the present invention, R⁶ is a 5-6 membered saturated or unsaturated heterocyclic ring system comprising 1-2 heteroatoms independently selected from N, O and S.

In a further embodiment of the present invention, R⁷ is H, C₁₋₆alkyl, C₃₋₇cycloalkyl, C₁₋₆alkyloxy, C₆₋₁₀arylC₁₋₂alkyloxy or halogen, said C₁₋₆alkyl, C₁₋₆alkyloxy and C₆₋₁₀arylC₁₋₂alkyloxy being optionally substituted with one or more halogens. In another embodiment, R⁷ is H, methyl, trifluoromethyl, ethyl, cyclopropyl, 2-methylpropyl, methoxy, bromo or chloro.

In a further embodiment of the present invention, R⁸ is H, C₁₋₆alkyl, C₃₋₇cycloalkyl, C₁₋₆alkyloxy, C₆₋₁₀arylC₁₋₂alkyloxy, NR¹⁰R¹¹ or halogen, said C₁₋₆alkyl, C₁₋₆alkyloxy and C₆₋₁₀arylC₁₋₂alkyloxy being optionally substituted with one or more halogens, wherein R¹⁰ and R¹¹ have the previously defined meanings. In another embodiment, R⁸ is H, methyl, trifluoromethyl, ethyl, cyclopropyl or N,N-dimethylamino.

In a further embodiment of the present invention, R⁹ is H, C₁₋₆alkyl, C₁₋₆alkyloxy or halogen, said C₁₋₆alkyl and C₁₋₆alkyloxy being optionally substituted with one or more halogens. In another embodiment, R⁹ is H, methyl, ethyl, methoxy, bromo or chloro.

In a further embodiment of the present invention is a tricyclic heterocyclic derivative having the Formula II

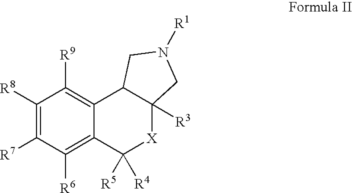

wherein X and R¹ and R³-R⁹ have the previously defined meanings.

In a further embodiment of the present invention is a tricyclic heterocyclic derivative having the Formula III

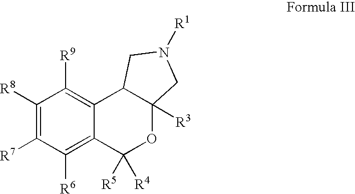

wherein R¹ and R³-R⁹ have the previously defined meanings.

In a further embodiment of the present invention is a tricyclic heterocyclic derivative having the Formula IV

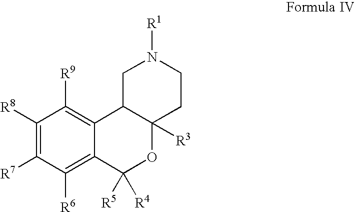

wherein R¹ and R³-R⁹ have the previously defined meanings.

In a further embodiment of the present invention is a tricyclic heterocyclic derivative having the Formula V

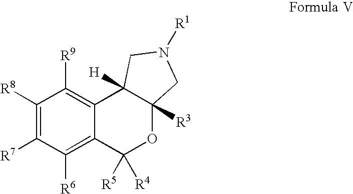

wherein R¹ and R³-R⁹ have the previously defined meanings.

In a further embodiment of the present invention is a tricyclic heterocyclic derivative having the Formula VI

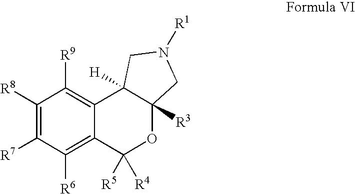

wherein R¹ and R³-R⁹ have the previously defined meanings.

In a further embodiment of the present invention is a tricyclic heterocyclic derivative selected from:

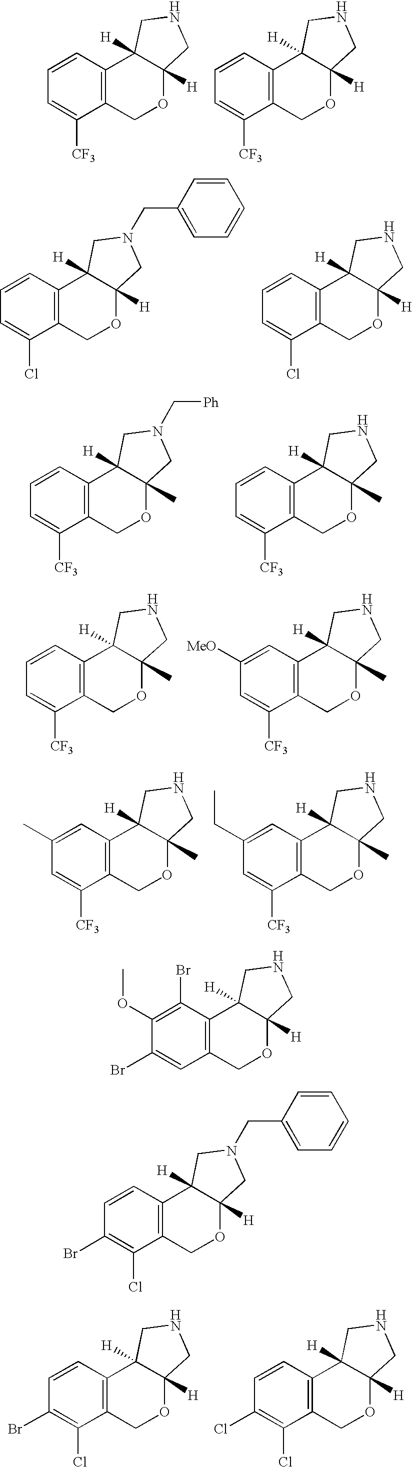

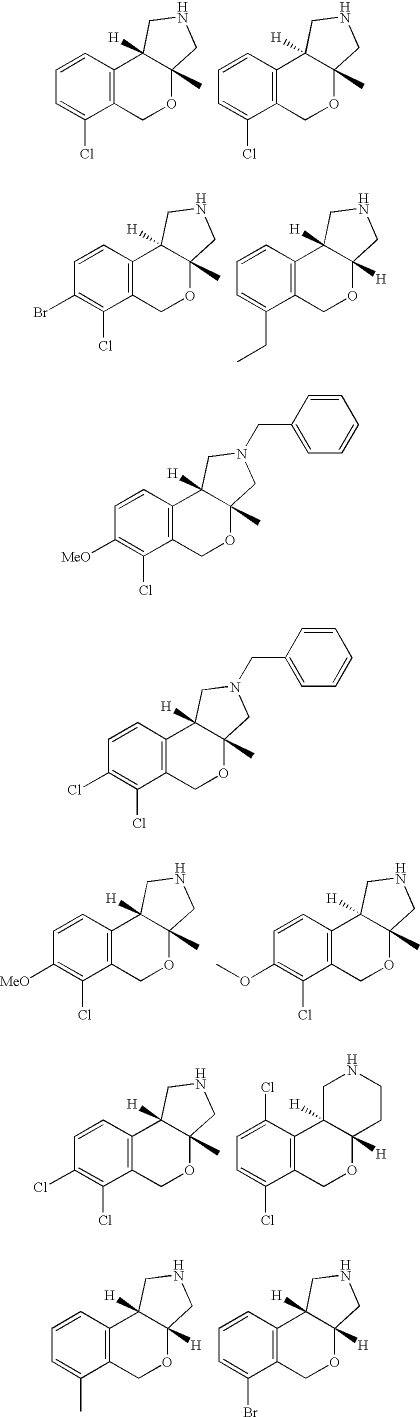

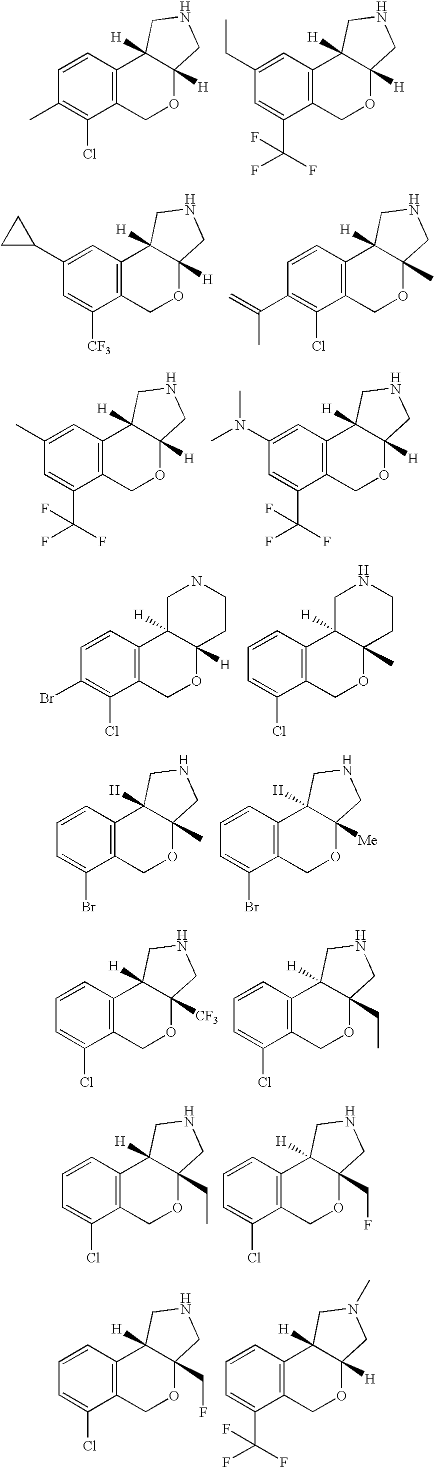

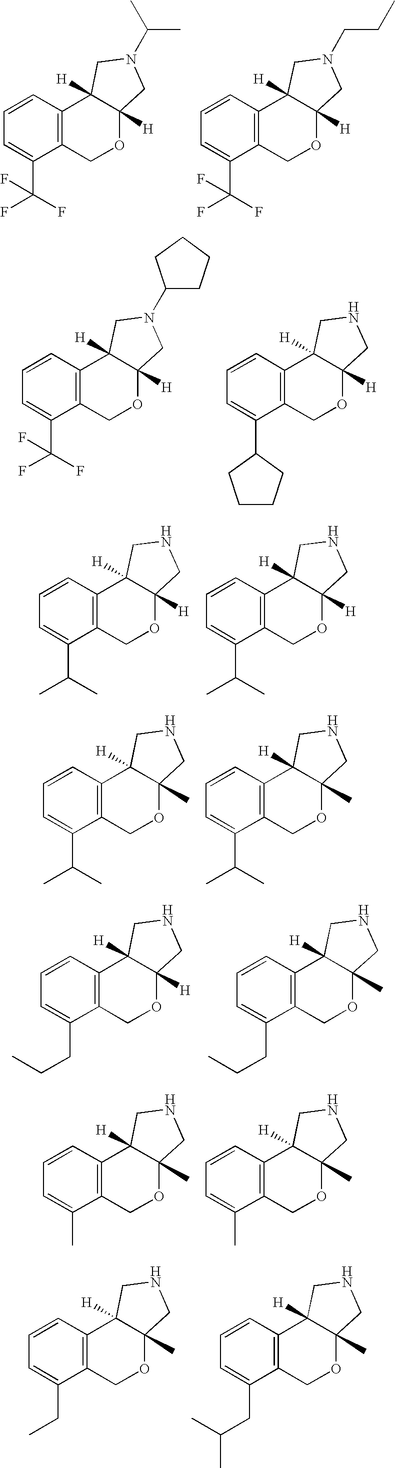

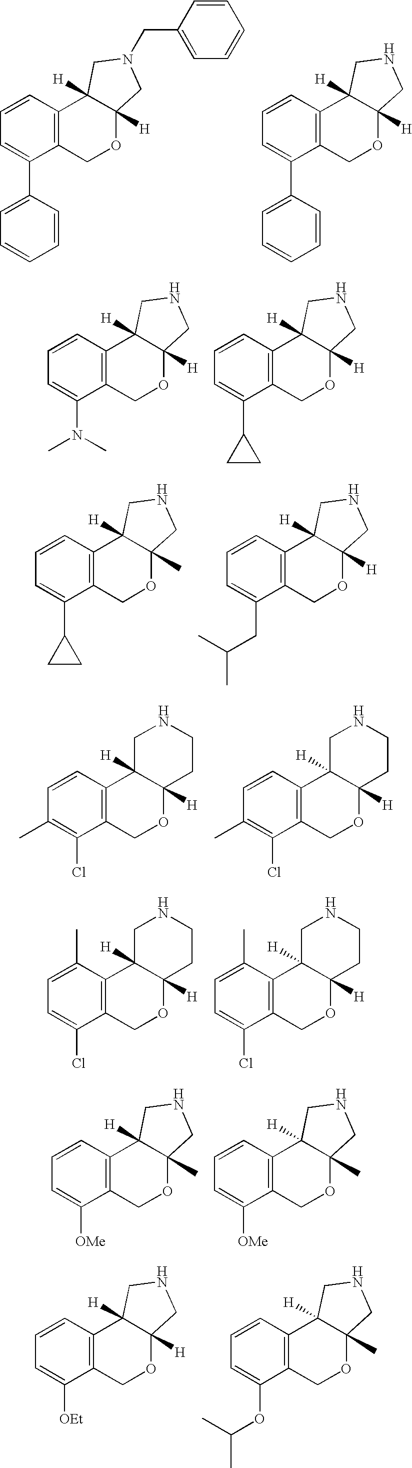

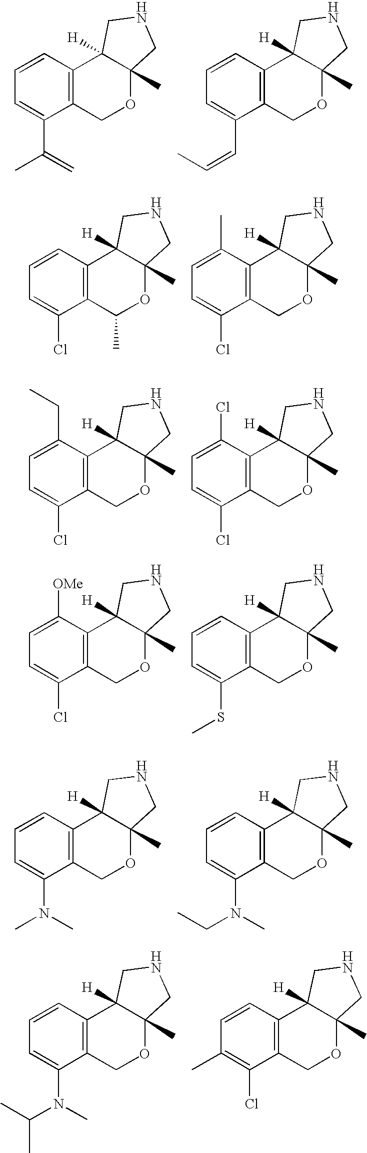

or a pharmaceutically acceptable salt or solvate thereof.

The tricyclic heterocyclic derivatives of Formula I-VI are prepared by methods well known in the art of organic chemistry, see for example, J. March, ‘Advanced Organic Chemistry’ 4th Edition, John Wiley and Sons. During synthetic sequences it may be necessary and/or desirable to protect sensitive or reactive groups on any of the molecules concerned. This is achieved by means of conventional protecting groups, such as those described in T. W. Greene and P. G. M. Wutts ‘Protective Groups in Organic Synthesis’ 3^{rd} Edition, John Wiley and Sons, 1999. The protective groups are optionally removed at a convenient subsequent stage using methods well known in the art.

The tricyclic heterocyclic derivatives (8) and (9) may be prepared, as shown in Scheme 1, from the appropriately substituted nitrile (1), wherein Y¹-Y⁴ and R⁴ and R⁵ have the previously defined meanings. The nitrile (1) can be readily hydrolysed using a suitable base, for example, potassium hydroxide in ethanol and water, to afford the acid (2). The substitution of the acid (2) can be modified by substituent directed halogenation. For example bromination of the acid (2) (wherein Y¹═CCl and Y²═Y³═Y⁴═CH) affords the acid (2) (wherein Y¹═CCl and Y² or Y⁴ can be mono-brominated, i.e. CBr). The bromine may then be converted to an alternative functional group or maintained for manipulation later in the synthesis. Coupling of acid (2) with a suitable protected amino alcohol (3) (wherein, for example R¹=Bn or alternative R¹ substituted amino alcohols, for example, wherein R¹═CH₃) affords the amide (4). The coupling reaction can be carried out using appropriate coupling reagents and conditions, for example cyclophos or 1-hydroxybezotriazole hydrate and N,N′-methanediylidenedipropan-2-amine. The relevant amino alcohols (3) (wherein R¹, R² and R³ have the previously defined meanings) are either commercially available or can readily be prepared using standard techniques well known in the art of organic chemistry. For example, 3-(benzylamino)propan-1-ol can be prepared by reductive amination of 3-aminopropan-1-ol with benzaldehyde using sodium triacetoxyborohydride. The alcohol (4) can be readily oxidized with the appropriate oxidation reagent (for example Dess-Martin periodinane) to afford the aldehyde (wherein R³═H) or ketone (wherein for example R³=alkyl) (5). Thermolysis of the benzocyclobutene aldehyde (wherein R³═H) or ketone (wherein, for example, R³=alkyl) (5) for example by heating in 1,2-dichlorobenzene, bromobenzene or 1,4-dioxane provides a mixture of the cis- and trans-intramolecular Diels-Alder products (6) and (7). For n=0, the trans-lactam (7) can be readily converted into the related cis-product (6) by treatment with the appropriate base (for example refluxing with DBN in toluene). The amines (8) and (9) can be obtained by reduction of the amides (6) and (7) using a suitable reducing agent (for example: LiAlH₄ or BH₃ DMS complex). The amines (8) and (9), wherein R¹═H, can be prepared by deprotection of the amines (8) and (9), wherein R¹ is benzyl. For example, such a benzyl protecting group can be removed by hydrogenation with palladium on carbon or by heating with 1-chloroethyl chloroformate and quenching with methanol. Alternatively, N-alkylation of the amines (8) and (9), wherein R¹ is H, with an appropriate alkylhalide (for example benzylbromide), or reductive amination with an appropriate aldehyde (for example formaldehyde) or ketone is also possible.

Derivatisation of compounds of Formula (6) and (7) substituted at positions Y¹, Y², Y³ or Y⁴ can be achieved by methods well known in the art of organic chemistry. For example, conversion of the lactams (6) and (7), wherein Y¹═CCl and R¹=Bn and Y²═Y³═Y⁴═CH into the corresponding lactams (6) and (7), wherein Y¹═CBr and R¹=Bn and Y²═Y³═Y⁴═CH can be achieved using nickel(II) bromide in DMF with heating. Similarly derivatisation of the amines (8) and (9) can be readily achieved using methods well known in the art of organic chemistry. For example, bromination of the amine (8) or (9) wherein Y¹═CCl and Y²═Y³═Y⁴═CH, using N-bromosuccinimide affords the amine (8) or (9), wherein Y¹═CCl, Y²═Y³═CH and Y⁴═CBr. Similarly, bromination of the amine (8) or (9) wherein Y¹═CCF₃ and Y²═Y³═Y⁴═CH using N-bromosuccinimide affords the amine (8) or (9), wherein Y¹═CCF₃, Y³═CBr and Y²═Y⁴═CH. The bromine may then be converted to an alternative functional group when R¹═H or when R¹ is suitably protected (for example Boc-protection).

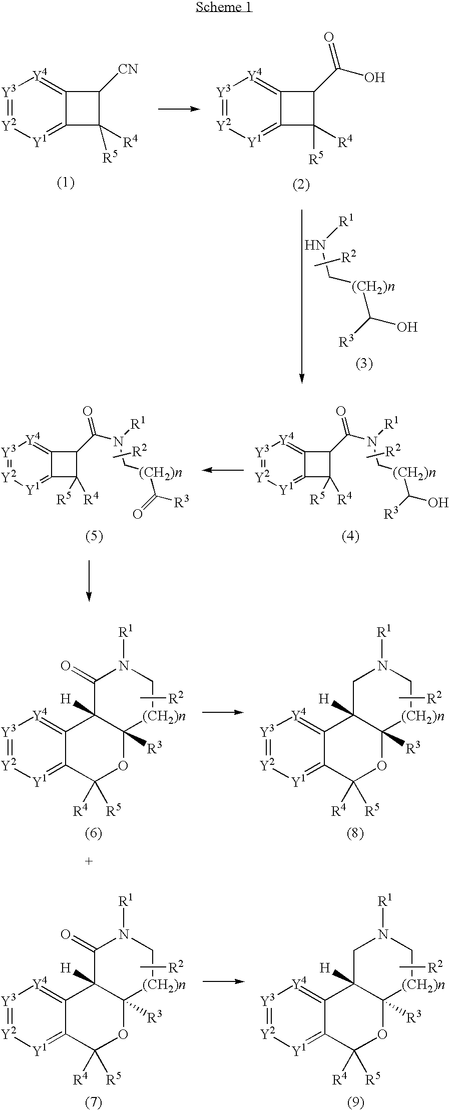

The cyclobutanenitrile (1), wherein R⁴ and R⁵ are H may be prepared, as shown in Scheme 2 from the appropriately substituted o-halo-benzaldehyde (10) (preferably wherein Hal=Br or Cl). For example, treatment of the o-halobenzaldehyde (10) with cyanoacetic acid, pyridine and ammonium acetate in toluene provides the corresponding cinnamonitrile (11), which is subsequently reduced (for example using NaBH₄) to give the dihydrocinnamonitrile (12). Decarboxylation by heating in the appropriate solvent (for example dimethyl acetamide) provides the nitrile (13). Subsequent ring closure of the nitrile (13) using sodium amide in ammonia affords the cyclobutane nitrile (1). Alternatively, the cyclobutane nitrile (1) can be readily prepared from the appropriately substituted m-halo-benzaldehyde (14) (preferably where Hal=Br or Cl). Treatment of the m-halobenzaldehyde (14) with cyanoacetic acid, pyridine and ammonium acetate in toluene provides the corresponding cinnamonitrile (15), which can subsequently be reduced (for example using NaBH₄) to give the dihydrocinnamonitrile (16). Decarboxylation by heating in the appropriate solvent (for example dimethyl acetamide) provides the nitrile (17) which in turn can be cyclised by, for example, treatment with sodium amide in ammonia to afford the cyclobutane nitrile (1).

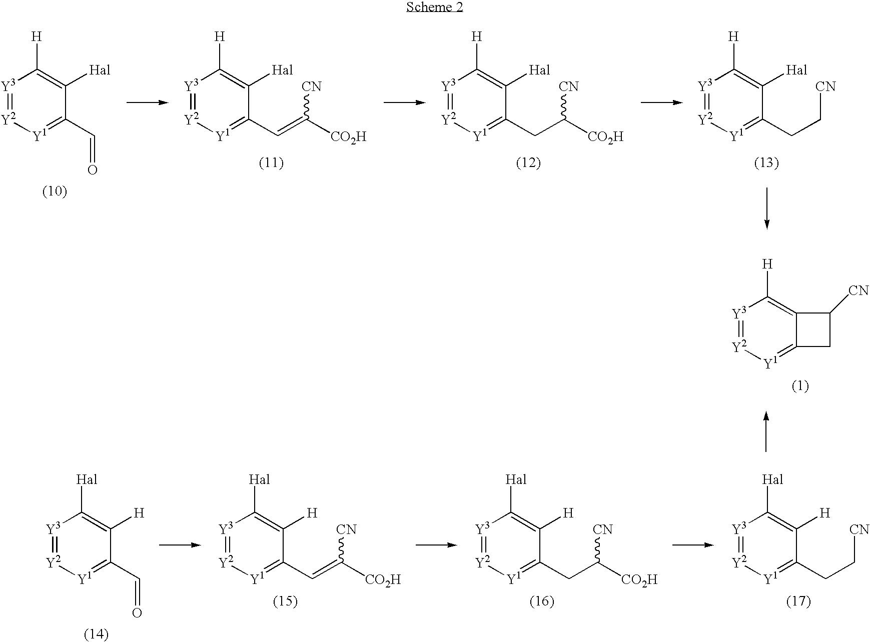

The nitrile (17) can be prepared from the nitrile (18), by direct halogenation. For example, when Y³═COMe, Y²═CH and Y¹═CMe halogenation with bromine in chloroform gives the brominated product (17) (see Scheme 3 below). Similarly, with the appropriate Y¹, Y² and Y³ substitutions, nitrile (13) can be prepared from the nitrile (18), by direct halogenation.

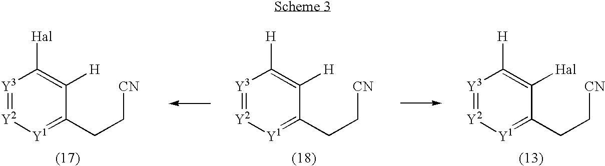

Alternatively, the nitrile (1) (wherein Y⁴≠CH), can be prepared by employing a [2+2] cycloaddition of the appropriate ketene (for example 1,1-dimethoxyethylene) with the appropriate benzyne generated by base (for example NaNH₂) induced dehalogenation (wherein Hal=Br or I) of halobenzene (19). The intermediated benzocyclobutenone ketal (20) can be hydrolysed under acidic conditions, for example, using aqueous hydrochloric acid in methanol, to afford benzocyclobutenone (21). The benzocyclobutenone (21) can subsequently be transformed into the cyclobutane nitrile (1) by, for example, reduction (for example using NaBH₄) to the intermediated alcohol which can be activated (for example by conversion to the corresponding mesylate) and treated with a suitable nitrile source (for example NaCN) to afford nitrile (1) (see Scheme 4 below).

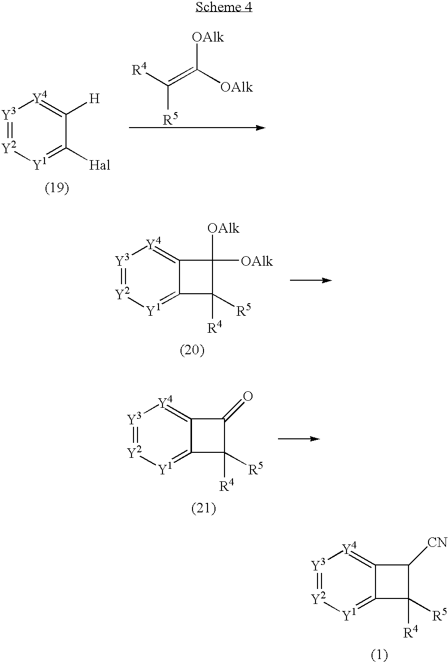

Compounds of Formula I (wherein, R⁴ or R⁵≠H) may be prepared by treatment of the protected amine (22) (for example wherein R¹═CO₂Et) with a suitable oxidising agent (for example Jones oxidation) to afford the intermediate ester (23). The alkylated products (24) are obtained by treatment of (23) with an excess or with one equivalent of a suitable alkylating reagent (for example R⁴Li or R⁵Li). Treatment with one equivalent of alkylating reagent affords an intermediate lactol which can be reduced (using for example trifluoroacetic acid and triethylsilane) then deprotected (using for example potassium hydroxide) to afford compounds of Formula I (wherein, R⁴ or R⁵≠H) (see Scheme 5 below).

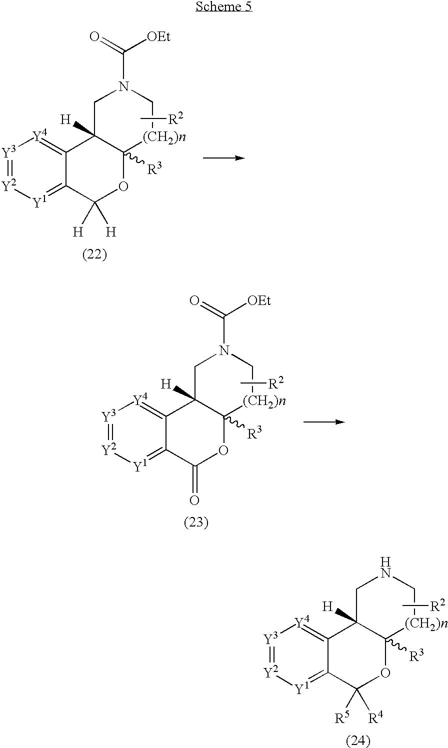

Compounds of Formula I, wherein X is O and m is 2, for example (27) and (28) may be prepared as shown in Scheme 6 from the appropriately substituted acid (2). Homologation of (2) can be achieved by conversion to the acid chloride using thionyl chloride and subsequent treatment with diazomethane to generate the alpha-diazoketone for subsequent Arndt-Eistert homologation using Ag(OBz)₂ and methanol to afford the ester (25). The ester (25) can then be hydrolysed to the homologated acid (26) using, for example, aqueous sodium hydroxide in ethanol. The synthetic protocol of scheme 1 is then followed to yield the products (27) and (28).

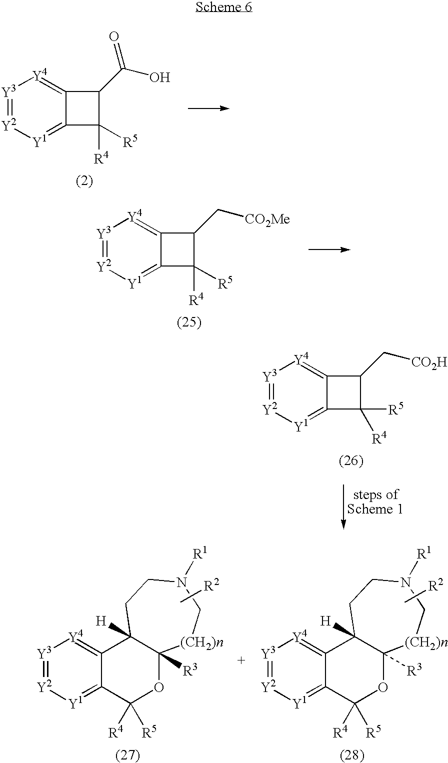

Compounds of Formula I wherein, X═OCR^{4′}R^{5′} for example (36) may be prepared from appropriate 2-methyl benzaldehyde derivatives (29) as shown is Scheme 7 for the trans-compounds and Scheme 8 for the cis-compounds. Reaction of the benzaldehyde (29) with the Wittig reagent (30) affords the alkene (31). Subsequent [3+2] cyclisation using, for example, the protected iminium (32) in the presence of trifluioroacetic acid affords the protected pyrollidine (33). Benzylic bromination, using for example N-bromosuccinimide provides the bromide (34). Ester reduction (using, for example, LiBH₄) and cyclisation (using for example sodium hydride in DMF) then affords the tricyclic product (35). N-deprotection (using for example 1-chloroethyl chloroformate, followed by methanol treatment) gives amine (36, R¹═H).

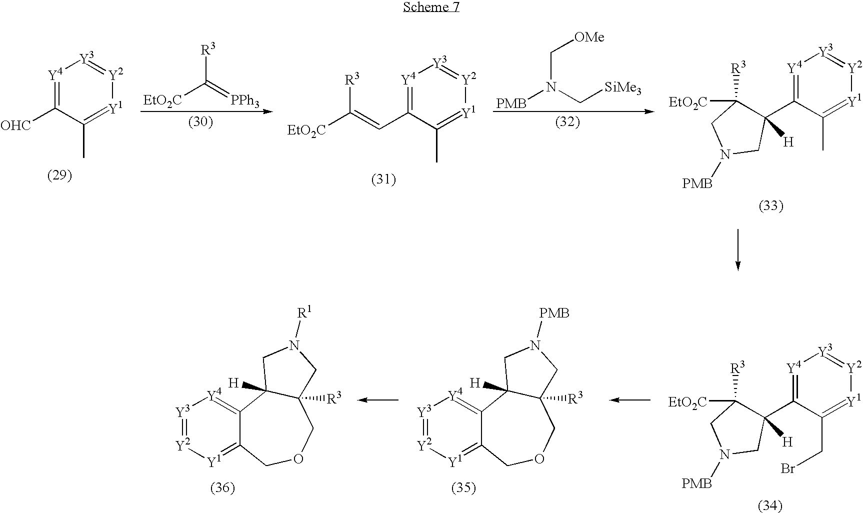

Alternatively reaction of (29) with Wittig reagent (37) generates the alkene (38). Subsequent [3+2] cyclisation using, for example, the protected iminium (32) in the presence of trifluioroacetic acid affords the protected pyrollidine (39). Alkylation of (39) using, for example, lithium diisopropyl amide in a suitable solvent, such as THF followed by treatment with the appropriate alkyl halide (R³-halide) provides the product (40). This is then brominated, the ester group reduced and the resulting alcohol cyclised as described above for conversion of (33) to (35). N-deprotection of the resulting tricyclic product (41) (using for example α-chloroethylchloroformate, followed by methanol treatment) gives amine (42, R¹═H) (Scheme 8).

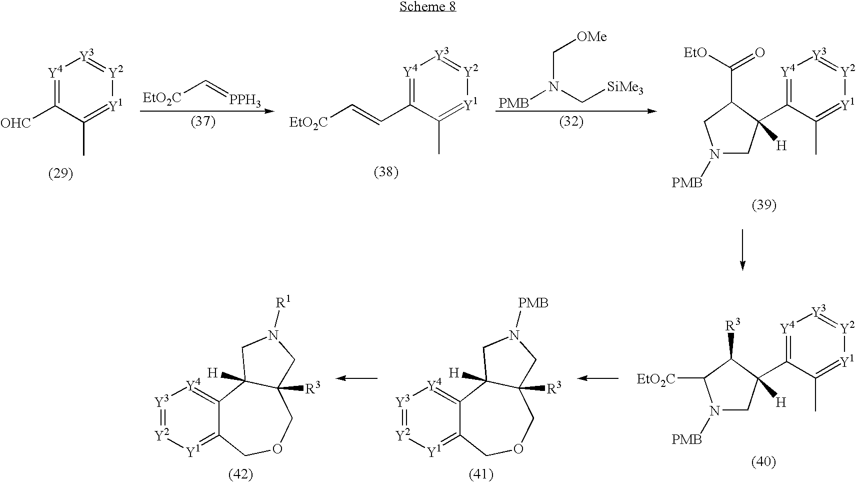

The present invention also includes within its scope all stereoisomeric forms of the tricyclic heterocyclic derivatives of Formula I resulting, for example, because of configurational isomerism. Such stereoisomeric forms are enantiomers, or diastereoisomers. For example, in the case where R² and R⁴ are H and R⁵ is methyl, the compound exists as diastereoisomers with three chiral centres. In the case of the individual enantiomers of tricyclic pyrrolidine or piperidine derivative of Formula I or salts or solvates thereof, the present invention includes the aforementioned stereoisomers substantially free, i.e., associated with less than 5%, preferably less than 2% and in particular less than 1% of the other enantiomer. Mixtures of stereoisomers in any proportion, for example a racemic mixture comprising substantially equal amounts of two enantiomers are also included within the scope of the present invention.

For chiral compounds, methods for asymmetric synthesis whereby the pure stereoisomers are obtained are well known in the art, e.g., synthesis with chiral induction, synthesis starting from chiral intermediates, enantioselective enzymatic conversions, separation of stereoisomers using chromatography on chiral media. Such methods are described in Chirality In Industry (edited by A. N. Collins, G. N. Sheldrake and J. Crosby, 1992; John Wiley). Likewise methods for synthesis of geometrical isomers are also well known in the art.

The present invention also includes within its scope a tricyclic heterocyclic derivative of Formula I in the form as a free base as well as in the form of a pharmaceutically acceptable salt. These salts are also obtained by treatment of said free base with an organic or inorganic acid such as hydrogen chloride, hydrogen bromide, hydrogen iodide, sulfuric acid, phosphoric acid, acetic acid, trifluoroacetic acid, propionic acid, glycolic acid, maleic acid, malonic acid, methanesulphonic acid, fumaric acid, succinic acid, tartaric acid, citric acid, benzoic acid and ascorbic acid. All salts, whether pharmaceutically acceptable or not are included within the scope of the present invention.

The tricyclic heterocyclic derivatives of the present invention also exists in amorphous forms. Multiple crystalline forms are also possible. All these physical forms are also included within the scope of the present invention.

Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS Pharm Sci Tech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The present invention also embraces isotopically-labelled compounds of the compounds described and claimed herein which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ¹⁸F, and ³⁶Cl, respectively.

Certain isotopically-labelled compounds of Formula I (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula I can generally be prepared by following procedures analogous to those disclosed in the Schemes and/or in the Examples hereinbelow, by substituting an appropriate isotopically labelled reagent for a non-isotopically labelled reagent.

Prodrugs of the compounds of the invention are also contemplated within the scope of the invention. A prodrug is a compound which acts as a drug precursor which, upon administration to a subject, undergoes conversion by metabolic or other chemical processes to yield a heterocyclic derivative of Formula I or a solvate or salt thereof. For example, where R¹ is H the nitrogen group may be capped as, for example, an amide or carbamate which upon administration to a subject will undergo conversion back to the free hydroxyl group. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

In a further aspect, the tricyclic heterocyclic derivatives of the present invention are useful in therapy. In particular, the tricyclic heterocyclic derivatives of the present invention are useful in therapy in humans or animals. As such, the tricyclic heterocyclic derivatives of the present invention are useful in the manufacture of a medicament for the treatment or prevention of diseases or disorders mediated by serotonin. In particular, the tricyclic heterocyclic derivatives of the present invention are useful in the manufacture of a medicament for the treatment or prevention of obesity, diabetes, diabetic complications, atherosclerosis, impared glucose tolerance and dyslipidemia, anxiety, depression, obsessive compulsive disorder, panic disorder, psychosis, schizophrenia, sleep disorder, sexual disorder and social phobias; cephalic pain; migraine and gastrointestinal disorders.

The present invention further includes a method for the treatment of a mammal, including a human, suffering from or liable to suffer from any of the aforementioned diseases or disorders, which method comprises administering an effective amount of a tricyclic heterocyclic derivative according to the present invention or a pharmaceutically acceptable salt or solvate thereof. By effective amount or therapeutically effective amount is meant an amount of compound or a composition of the present invention effective in inhibiting the above-noted diseases and thus producing the desired therapeutic, ameliorative, inhibitory or preventative effect.

The amount of a tricyclic heterocyclic derivative of the present invention or a pharmaceutically acceptable salt or solvate thereof, also referred to herein as the active ingredient, which is required to achieve a therapeutic effect will, of course, vary with the particular compound, the route of administration, the age and condition of the recipient, and the particular disorder or disease being treated.

A suitable daily dose for any of the above mentioned disorders will be in the range of 0.001 to 50 mg per kilogram body weight of the recipient (e.g. a human) per day, preferably in the range of 0.01 to 20 mg per kilogram body weight per day. The desired dose may be presented as multiple sub-doses administered at appropriate intervals throughout the day.

Whilst it is possible for the active ingredient to be administered alone, it is preferable to present it as a pharmaceutical formulation. The present invention therefore also provides a pharmaceutical composition comprising a tricyclic heterocyclic derivative according to the present invention in admixture with one or more pharmaceutically acceptable excipients, such as the ones described in Gennaro et. al., Remmington: The Science and Practice of Pharmacy, 20^{th} Edition, Lippincott, Williams and Wilkins, 2000; see especially part 5: pharmaceutical manufacturing. The term “acceptable” means being compatible with the other ingredients of the composition and not deleterious to the recipients thereof. Suitable excipients are described e.g., in the Handbook of Pharmaceutical Excipients, 2^{nd} Edition; Editors A. Wade and P. J. Weller, American Pharmaceutical Association, Washington, The Pharmaceutical Press, London, 1994. Compositions include those suitable for oral, nasal, topical (including buccal, sublingual and transdermal), parenteral (including subcutaneous, intravenous and intramuscular) or rectal administration.

The mixtures of a tricyclic heterocyclic derivative according to the present invention and one or more pharmaceutically acceptable excipient or excipients may be compressed into solid dosage units, such as tablets, or be processed into capsules or suppositories. By means of pharmaceutically suitable liquids the tricyclic heterocyclic derivatives of the present invention can also be applied as an injection preparation in the form of a solution, suspension, emulsion, or as a spray, e.g., a nasal or buccal spray. For making dosage units e.g., tablets, the use of conventional additives such as fillers, colorants, polymeric binders and the like is contemplated. In general, any pharmaceutically acceptable additive can be used. The tricyclic heterocyclic derivatives of the present invention are also suitable for use in an implant, a patch, a gel or any other preparation for immediate and/or sustained release.

Suitable fillers with which the pharmaceutical compositions can be prepared and administered include lactose, starch, cellulose and derivatives thereof, and the like, or mixtures thereof used in suitable amounts. For parenteral administration, aqueous suspensions, isotonic saline solutions and sterile injectable solutions may be used, containing pharmaceutically acceptable dispersing agents and/or wetting agents, such as propylene glycol or butylene glycol.

The present invention further includes a pharmaceutical composition, as hereinbefore described, in combination with packaging material suitable for said composition, said packaging material including instructions for the use of the composition for the use as hereinbefore described.

The present invention is further illustrated by the following examples which are not intended to limit the scope thereof. Unless indicated otherwise, percent is percent by weight given the component and the total weight of the composition, temperature is in ° C. or is at ambient temperature, and pressure is at or near atmospheric. Commercial reagents were used without further purification.

### Methods

General Chemical Procedures. All reagents were either purchased from common commercial sources or synthesised according to literature procedures using commercial sources.

All NMR spectra were recorded using a Bruker AC400 spectrometer. Chemical shifts were recorded in parts per million using TMS as a standard. Mass spectra were recorded on a Shimadzu LC-8A (HPLC) PE Sciex API 150EX LCMS. Analytical reversed-phase LCMS analysis was carried out on LUNA C18 column (5μ; 30×4.6 mm) under gradient conditions (100% water/0.1% formic acid to 100% acetonitrile/0.1% formic acid) at a flow rate of 3 mL/min.

For chromatography eluent: x-y % solvent A in solvent B means that a gradient of the eluent of x % (v/v) of solvent A in solvent B to y % (v/v) of solvent A in solvent B was used.

### Abbreviations

Dimethylformamide (DMF), dimethylacetamide (DMA), 1,2-dimethoxyethane (DME), dichloromethane (DCM), dimethylsuphoxide (DMSO), tetrahydrofuran (THF), high pressure liquid chromatography (HPLC), diisopropylethylamine (DIPEA), triethylamine (TEA), trifluoroacetic acid (TFA), tert-butyloxycarbonyl (Boc), dimethylsulphide (DMS), diaza-1,5-bicyclo[4,3,0]non-5-ene (DBN), p-methoxybenzyl (PMB), N-methylpyrrolidinone (NMP), 50 wt. % solution of propylphosphonic anhydride in EtOAc (cyclophos), diethylamine (DEA), iso-propylamine (IPam) and benzyl (Bn).

### EXAMPLE 1

### cis-2-Benzyl-6-(trifluoromethyl)-1,2,3,3a,5,9b-hexahydroisochromeno[3,4-c]pyrrole & trans-2-benzyl-6-(trifluoromethyl)-1,2,3,3a,5,9b-hexahydroisochromeno[3,4-c]pyrrole

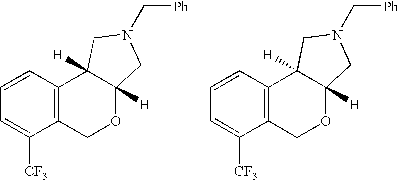

### 1.1 Preparation of 3-(2-chloro-6-(trifluoromethyl)phenyl)-2-cyanoacrylic acid

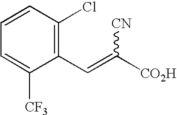

A stirred mixture of 2-chloro-6-(trifluoromethyl)benzaldehyde (240 mmol, 50 g), cyanoacetic acid (240 mmol, 20.39 g), ammonium acetate (47.9 mmol, 3.70 g), pyridine (420 mmol, 33.2 g) and toluene (184 ml) was refluxed using a Dean Stark apparatus until one molar equivalent of water was collected. The reaction mixture was allowed to cool and then concentrated to form a residue that was then stirred with 10% aqueous HCl. The product was extracted with ethyl acetate, dried (Na₂SO₄) and concentrated to afford a crude solid on standing. Recrystallisation from toluene afforded 3-(2-chloro-6-(trifluoromethyl)phenyl)-2-cyanoacrylic acid (77% yield), ¹H NMR (400 MHz, CDCl₃) ppm 8.75 (1H, s (br), CO₂H), 8.47 (1H, s, CHCCO₂H), 7.74 (1H, d, ArH), 7.72 (1H, d, ArH), 7.56 (1H, t, ArH).

### 1.2 Preparation of 3-(2-chloro-6-(trifluoromethyl)phenyl)-2-cyanopropanoic acid

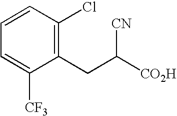

Sodium borohydride (805 mmol, 30.5 g) was added dropwise over a period of two hours to a stirred mixture of 3-(2-chloro-6-(trifluoromethyl)phenyl)-2-cyanoacrylic acid (218 mmol, 59.99 g) in aqueous saturated sodium hydrogen carbonate (200 ml) and methanol (605 ml) cooled to about 15° C. The reaction mixture was allowed to warm up to room temperature and it was stirred at room temperature for 30 minutes then concentrated under reduced pressure. The residue was partitioned between water and ether. The aqueous layer was acidified and extracted with ether. The organic phase was dried over sodium sulphate, filtered and the filtrate was evaporated under reduced pressure to afford 3-(2-chloro-6-(trifluoromethyl)phenyl)-2-cyanopropanoic acid (87% yield), ¹H NMR (400 MHz, CD₃OD) ppm 7.76 (1H, d, ArH), 7.74 (1H, d, ArH), 7.51 (1H, t, ArH), 4.33 (1H, t, CNCHCO₂H), 3.57 (2H, d, CH₂).

### 1.3 Preparation of 3-(2-chloro-6-(trifluoromethyl)phenyl)propanenitrile

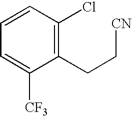

3-(2-Chloro-6-(trifluoromethyl)phenyl)-2-cyanopropanoic acid (189 mmol, 52.41 g) was dissolved in DMA (79 ml) and heated for an hour and a half at 140-150° C. After cooling, the reaction mixture was poured into water and extracted with ether. The ether layer was washed with a saturated solution of sodium hydrogen carbonate and then with brine. The organic phase was dried (Na₂SO₄) and concentrated in vacuo to afford 3-(2-chloro-6-trifluoromethyl)phenyl)propanenitrile (92% yield), ¹H NMR (400 MHz, CD₃OD) ppm 7.73 (1H, d, ArH), 7.70 (1H, d, ArH), 7.47 (1H, t, ArH), 3.30 (2H, t, ArCH₂CH₂CN), 2.76 (2H, t, ArCH₂CH₂CN).

### 1.4 Preparation of 3-(trifluoromethyl)-1,2-dihydrocyclobutabenzene-1-carbonitrile

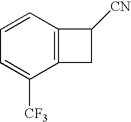

Ammonia gas was condensed to the required volume (˜260 mL). Commercial NaNH₂ (252 mmol, 9.84 g) was added to the ammonia at −78° C. and after stirring for 10 minutes 3-(2-chloro-6-(trifluoromethyl)phenyl)propanenitrile (64.2 mmol, 15 g) was added over 5 minutes. The mixture was allowed to warm such that the resultant mixture was stirred at reflux for 3 h before being neutralised with solid ammonium nitrate (278 mmol, 22.25 g) and allowed to stand overnight under a flow of nitrogen. All the ammonia evaporated and water was added to the solid residue and the products was extracted with dichloromethane (×3). The combined organics were washed with dilute hydrochloric acid (5%), followed by water. The organics were dried (Na₂SO₄) and concentrated to afford a residue. Flash chromatography of the residue using ethyl acetate in heptane (5% to 40%) as the eluent to afford 3-(trifluoromethyl)-1,2-dihydrocyclobutabenzene-1-carbonitrile (57.9% yield), ¹H NMR (400 MHz, CD₃OD) ppm 7.61-7.49 (3H, m, 3×ArH), 4.57 (1H, dd, CHCN), 3.86 (1H, dd, CH₂), 3.62 (1H, dd, CH₂).

### 1.5 Preparation of 3-(trifluoromethyl)-1,2-dihydrocyclobutabenzene-1-carboxylic acid

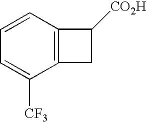

A solution of 3-(trifluoromethyl)-1,2-dihydrocyclobutabenzene-1-carbonitrile (2.86 mmol, 563 mg) and potassium hydroxide (14.28 mmol, 801 mg) in ethanol (9.33 ml) and water (1.87 ml) was refluxed for 2 h. After evaporation of the solvent under reduced pressure, the aqueous residue was washed with ether. The organic layer was extracted with 2N NaOH (aq) and the combined aqueous layers were acidified with 5 N HCl then extracted with ether. The extracts were washed with brine, dried (Na₂SO₄) and concentrated in vacuo. The residue was washed with heptane and ether (5:1) to afford 3-(trifluoromethyl)-1,2-dihydrocyclobutabenzene-1-carboxylic acid (83% yield), ¹H NMR (400 MHz, CD₃OD) ppm 7.48-7.37 (3H, m, 3×ArH), 4.42 (1H, dd, CHCO₂H), 3.62-3.49 (2H, m, CH₂).

### 1.6 Preparation of N-benzyl-N-(2-hydroxyethyl)-3-(trifluoromethyl)-1,2-dihydrocyclobutabenzene-1-carboxamide

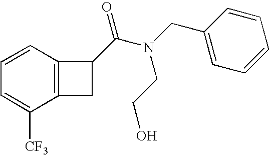

A mixture of 2-(benzylamino)ethanol (45.3 mmol, 6.44 ml, 6.85 g), triethylamine (60.4 mmol, 8.42 ml, 6.11 g), cyclophos (36.2 mmol, 21.57 ml in ethyl acetate) and 3-(trifluoromethyl)-1,2-dihydrocyclobutabenzene-1-carboxylic acid (30.2 mmol, 6.527 g) in dichloromethane was stirred at rt for 2 h. The reaction mixture was partitioned between dichloromethane and 2 N HCl. The organic layer was washed with water, then brine, dried (Na₂SO₄) and concentrated in vacuo. Flash chromatography of the residue using ethyl acetate in heptane (20% to 100%) afforded N-benzyl-N-(2-hydroxyethyl)-3-(trifluoromethyl)-1,2-dihydrocyclobutabenzene-1-carboxamide (60% yield), EI-MS: m/z=350.5 [M+H]⁺.

### 1.7 Preparation of N-benzyl-N-(2-oxoethyl)-3-(trifluoromethyl)-1,2-dihydrocyclobutabenzene-1-carboxamide

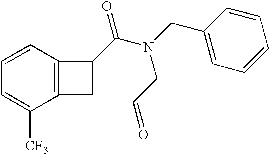

To a solution of N-benzyl-N-(2-hydroxyethyl)-3-(trifluoromethyl)-1,2-dihydrocyclobutabenzene-1-carboxamide (1.717 mmol, 600 mg) in dichloromethane (2 ml) was added a solution of Dess-Martin periodinane (1.717 mmol, 728 mg, 4.86 ml) 15 wt % in dichloromethane. The mixture was stirred at room temperature for 2 h and then saturated aqueous NaHCO₃ was added and the mixture stirred for a further 30 min. The mixture was then extracted with dichloromethane (×3), washed with brine, dried (MgSO₄) and concentrated under reduced pressure to afford a residue. Immediate flash chromatography using ethyl acetate in heptane (50%) as the eluent afforded N-benzyl-N-(2-oxoethyl)-3-(trifluoromethyl)-1,2-dihydrocyclobutabenzene-1-carboxamide (75% yield), EI-MS: m/z=348.3 [M+H]⁺.

### 1.8 Preparation of trans-2-benzyl-6-(trifluoromethyl)-3,3a,5,9b-tetrahydroisochromeno[3,4-c]pyrrol-1(2H)-one & cis-2-benzyl-6-(trifluoromethyl)-3,3a,5,9b-tetrahydroisochromeno[3,4-c]pyrrol-1(2H)-one

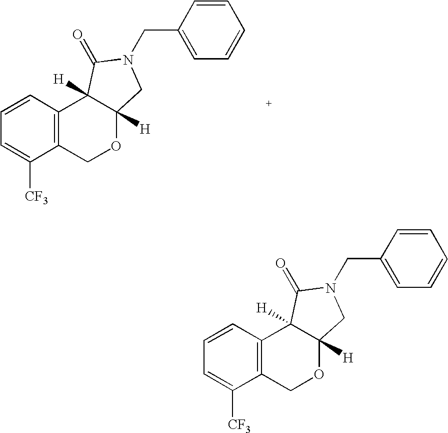

N-Benzyl-N-(2-oxoethyl)-3-(trifluoromethyl)-1,2-dihydrocyclobutabenzene-1-carboxamide (1.296 mmol, 450 mg) was placed into a microwave vial together with o-dichlorobenzene (6 ml). The mixture was microwaved at 210° C. for 30 min. The mixture was flash chromatographed using heptane, then ethyl acetate in heptane (10%, 20% & 50%) to afford trans-2-benzyl-6-(trifluoromethyl)-3,3a,5,9b-tetrahydroisochromeno[3,4-c]pyrrol-1(2H)-one (41% yield), EI-MS: m/z=348.1 [M+H]⁺, followed by cis-2-benzyl-6-(trifluoromethyl)-3,3a,5,9b-tetrahydroisochromeno[3,4-c]pyrrol-1(2H)-one (28% yield), EI-MS: m/z=348.4 [M+H]⁺.

### 1.9 Preparation of cis-2-benzyl-6-(trifluoromethyl)-1,2,3,3a,5,9b-hexahydroisochromeno[3,4-c]pyrrole

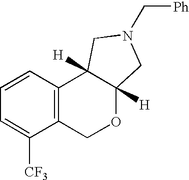

Borane-dimethyl sulfide complex (25.4 mmol, 2.442 ml, 1.929 g) was added to a solution of cis-2-benzyl-6-(trifluoromethyl)-3,3a,5,9b-tetrahydroisochromeno[3,4-c]pyrrol-1(2H)-one (1.26 g, 3.63 mmol) in tetrahydrofuran (40 ml). The mixture was refluxed under nitrogen for 6 h then cooled to 5° C. and 6N aqueous HCl (12 ml) was added. The mixture was refluxed for an additional 1.5 h then cooled to room temperature. The mixture was concentrated in vacuo and the residue was loaded onto a pre-acidified SCX column and flushed with MeOH. The SCX column was then flushed with 2M NH₃ in methanol and the eluent concentrated in vacuo to afford cis-2-benzyl-6-(trifluoromethyl)-1,2,3,3a,5,9b-hexahydroisochromeno[3,4-c]pyrrole (590 mg), EI-MS: m/z=334.0 [M+H]⁺.

### 1.10 Preparation of trans-2-benzyl-6-(trifluoromethyl)-1,2,3,3a,5,9b-hexahydroisochromeno[3,4-c]pyrrole

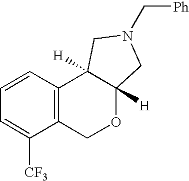

Similar reaction conditions to that in procedure 1.9 were repeated for trans-2-benzyl-6-(trifluoromethyl)-3,3a,5,9b-tetrahydroisochromeno[3,4-c]pyrrol-1(2H)-one to afford trans-2-benzyl-6-(trifluoromethyl)-1,2,3,3a,5,9b-hexahydroisochromeno[3,4-c]pyrrole, EI-MS: m/z=334.0 [M+H]⁺.

### EXAMPLE 2

### cis-6-(Trifluoromethyl)-1,2,3,3a,5,9b-hexahydroisochromeno[3,4-c]pyrrole

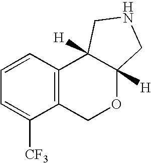

A solution of cis-2-benzyl-6-(trifluoromethyl)-1,2,3,3a,5,9b-hexahydroisochromeno[3,4-c]pyrrole (1.800 mmol, 600 mg) and 1-chloroethyl chloroformate (9.00 mmol, 971 μl, 1287 mg) in toluene (2 ml) was subjected to microwave irradiation at 160° C. for 15 minutes, then methanol (2 ml) was added to the mixture and the mixture was subjected to microwave irradiation at 160° C. for 5.5 minutes. The mixture was then concentrated and loaded onto a pre-acidified SCX column using methanol. The product was eluted with 2M ammonia in methanol and then concentrated to afford the desired product and starting material (400 mg). Flash chromatography using 5 to 10% MeOH in DCM (1% ammonium hydroxide was added) as the eluent afforded cis-2-benzyl-6-(trifluoromethyl)-1,2,3,3a,5,9b-hexahydroisochromeno[3,4-c]pyrrole (50 mg), followed by cis-6-(trifluoromethyl)-1,2,3,3a,5,9b-hexahydroisochromeno[3,4-c]pyrrole (80% yield), EI-MS: m/z=244.4 [M+H]⁺.

### EXAMPLE 3

### trans-6-(Trifluoromethyl)-1,2,3,3a,5,9b-hexahydroisochromeno[3,4-c]pyrrole

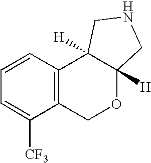

Similar reaction conditions to that in the procedure of Example 2 were repeated for trans-2-benzyl-6-(trifluoromethyl)-1,2,3,3a,5,9b-hexahydroisochromeno[3,4-c]pyrrole (63 mg) to afford trans-6-(trifluoromethyl)-1,2,3,3a,5,9b-hexahydroisochromeno[3,4-c]pyrrole (13% yield), EI-MS: m/z=244.4 [M+H]⁺.

### EXAMPLE 4

### cis-2-Benzyl-6-chloro-1,2,3,3a,5,9b-hexahydroisochromeno[3,4-c]pyrrole

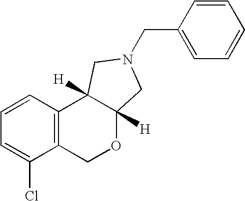

### 4.1 Preparation of N-benzyl-3-chloro-N-(2-oxoethyl)-1,2-dihydrocyclobutabenzene-1-carboxamide

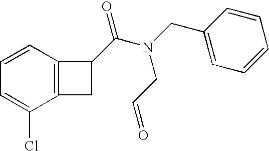

N-Benzyl-3-chloro-N-(2-oxoethyl)-1,2-dihydrocyclobutabenzene-1-carboxamide was prepared following a similar protocol to procedure 1.1 to 1.7, starting with 2,6-dichlorobenzaldehyde.

### 4.2 Preparation of cis-2-benzyl-6-chloro-3,3a,5,9b-tetrahydroisochromeno[3,4-c]pyrrol-1(2H)-one and trans-2-benzyl-6-chloro-3,3a,5,9b-tetrahydro isochromeno[3,4-c]pyrrol-1(2H)-one

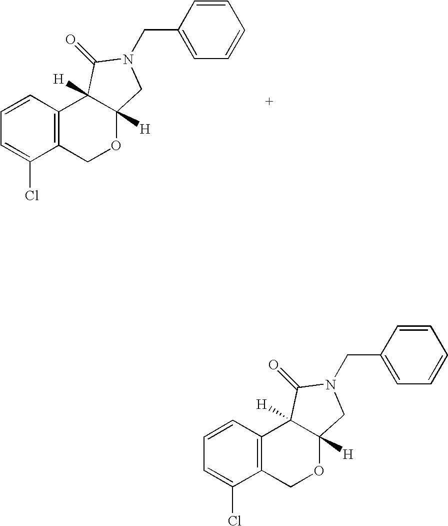

The title compounds were prepared following a similar protocol to procedure 1.8 using N-benzyl-3-chloro-N-(2-oxoethyl)-1,2-dihydrocyclobutabenzene-1-carboxamide (7.01 mmol, 2.2 g) affording trans-2-benzyl-6-chloro-3,3a,5,9b-tetrahydroisochromeno[3,4-c]pyrrol-1(2H)-one (39% yield), EI-MS: m/z=314.3 [M+H]⁺; followed by cis-2-benzyl-6-chloro-3,3a,5,9b-tetrahydroisochromeno[3,4-c]pyrrol-1(2H)-one (47% yield), EI-MS: m/z=314.0 [M+H]⁺.

### 4.3 Preparation of cis-2-benzyl-6-chloro-1,2,3,3a,5,9b-hexahydroisochromeno[3,4-c]pyrrole

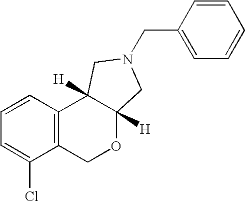

The title compound was prepared according to procedure 1.9 using cis-2-benzyl-6-chloro-3,3a,5,9b-tetrahydroisochromeno[3,4-c]pyrrol-1(2H)-one (1.593 mmol, 500 mg) to afford cis-2-benzyl-6-chloro-1,2,3,3a,5,9b-hexahydroisochromeno[3,4-c]pyrrole (72% yield), EI-MS: m/z=300.4 [M+H]⁺.

### EXAMPLE 5

### cis-6-Chloro-1,2,3,3a,5,9b-hexahydroisochromeno[3,4-c]pyrrole hydrochloride

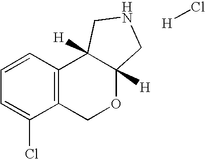

Similar reaction conditions to the procedure of Example 2 were repeated for cis-2-benzyl-6-chloro-1,2,3,3a,5,9b-hexahydroisochromeno[3,4-c]pyrrole (0.133 mmol, 40 mg) to afford cis-6-chloro-1,2,3,3a,5,9b-hexahydroisochromeno[3,4-c]pyrrole hydrochloride (41%), EI-MS: m/z=210.1 [M+H]⁺.

### EXAMPLE 6

### trans-6-Chloro-1,2,3,3a,5,9b-hexahydroisochromeno[3,4-c]pyrrole hydrochloride

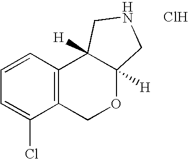

Similar procedures to those described in Examples 1 and 2 were employed, using trans-2-benzyl-6-chloro-3,3a,5,9b-tetrahydroisochromeno[3,4-c]pyrrol-1(2H)-one to afford trans-6-chloro-1,2,3,3a,5,9b-hexahydroisochromeno[3,4-c]pyrrole hydrochloride, EI-MS: m/z=210.1 [M+H]⁺.

### EXAMPLE 7

### cis-2-Benzyl-3a-methyl-6-(trifluoromethyl)-1,2,3,3a,5,9b-hexahydroisochromeno[3,4-c]pyrrole & trans-2-benzyl-3a-methyl-6-(trifluoromethyl)-1,2,3,3a,5,9b-hexahydro isochromeno[3,4-c]pyrrole

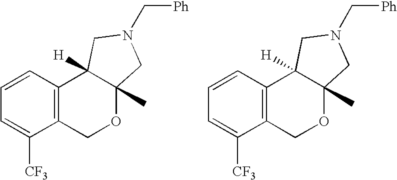

### 7.1 Preparation of N-benzyl-N-(2-hydroxypropyl)-3-(trifluoromethyl)-1,2-dihydrocyclobutabenzene-1-carboxamide

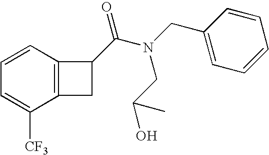

Methylmagnesium bromide (2.344 mmol) was added to a solution of N-benzyl-N-(2-oxoethyl)-3-(trifluoromethyl)-1,2-dihydrocyclobutabenzene-1-carboxamide (740 mg, 2.13 mmol) in tetrahydrofuran (8 ml) at 0° C. The reaction was stirred for 2 h then quenched with water and NH₄Cl (aq). The product was extracted with ethyl acetate (×2). The organic layers were dried (Na₂SO₄) and concentrated in vacuo. Flash chromatography of the residue using ethyl acetate in heptane (10% to 40%) as the eluent gave N-benzyl-N-(2-hydroxypropyl)-3-(trifluoromethyl)-1,2-dihydrocyclobutabenzene-1-carboxamide (51.7% yield), EI-MS: m/z=364.6 [M+H]⁺.

### 7.2 Preparation of N-benzyl-N-(2-oxopropyl)-3-(trifluoromethyl)-1,2-dihydrocyclobutabenzene-1-carboxamide

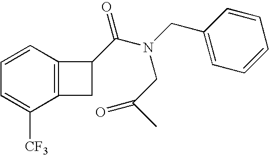

Dess-Martin periodinane (9.91 mmol, 3.09 ml, 4202 mg) 15 wt % in dichloromethane was added to a solution of N-benzyl-N-(2-hydroxypropyl)-3-(trifluoromethyl)-1,2-dihydrocyclobutabenzene-1-carboxamide (1.101 mmol, 400 mg) in dichloromethane (10 ml). The mixture was stirred at room temperature for 2 h and then saturated aqueous NaHCO₃ was added and the mixture stirred for a further 30 min. The mixture was then filtered, extracted with dichloromethane (×3), washed with brine, dried (MgSO₄) and concentrated under reduced pressure to afford a residue. Flash chromatography using ethyl acetate in heptane (10% to 30%) as the eluent afforded N-benzyl-N-(2-oxopropyl)-3-(trifluoromethyl)-1,2-dihydrocyclobutabenzene-1-carboxamide (78% yield), EI-MS: m/z=362.4 [M+H]⁺.

### 7.3 Preparation of trans-2-benzyl-3a-methyl-6-(trifluoromethyl)-3,3a,5,9b-tetrahydroisochromeno[3,4-c]pyrrol-1(2H)-one & cis-2-benzyl-3a-methyl-6-(trifluoromethyl)-3,3a,5,9b-tetrahydroisochromeno[3,4-c]pyrrol-1(2H)-one

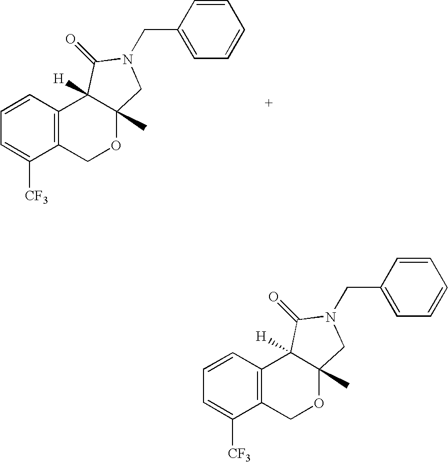

N-Benzyl-N-(2-oxopropyl)-3-(trifluoromethyl)-1,2-dihydrocyclobutabenzene-1-carboxamide (0.858 mmol, 310 mg) was placed into a microwave vial together with o-dichlorobenzene (3 ml). The mixture was microwaved at 220° C. for 110 min. The mixture was loaded onto a column and flash chromatographed using heptane, then ethyl acetate in heptane (10% to 50%) as the eluent to afford trans-2-benzyl-3a-methyl-6-(trifluoromethyl)-3,3a,5,9b-tetrahydroisochromeno[3,4-c]pyrrol-1(2H)-one (60 mg); ¹H NMR (400 MHz, CDCl₃) ppm 8.45 (1H, d, ArH), 7.59 (1H, d, ArH), 7.20-7.45 (6H, m, ArH), 5.16 (1H, d, CH₂O), 5.05 (1H, d, CH₂O), 4.56 (1H, d, CH₂N), 4.48 (1H, d, CH₂N), 3.72 (1H, s, CHCON), 3.40 (1H, d, CH₂N), 3.12 (1H, d, CH₂N) and 0.98 (3H, s, CH₃), followed by cis-2-benzyl-3a-methyl-6-(trifluoromethyl)-3,3a,5,9b-tetrahydroisochromeno[3,4-c]pyrrol-1(2H)-one (45 mg); ¹H NMR (400 MHz, CDCl₃) ppm 7.75 (1H, d, ArH), 7.58 (1H, d, ArH), 7.41 (1H, t, ArH), 7.20-7.39 (5H, m, ArH), 4.95 (1H, d, CH₂O), 4.88 (1H, d, CH₂O), 4.59 (1H, d, CH₂N), 4.45 (1H, d, CH₂N), 3-30-3.40 (3H, m, CHCON & CH₂N) and 1.44 (3H, s, CH₃).

### 7.4 Preparation of cis-2-benzyl-3a-methyl-6-(trifluoromethyl)-1,2,3,3a,5,9b-hexahydroisochromeno[3,4-c]pyrrole

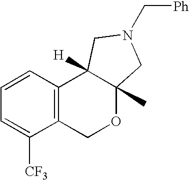

The title compound was prepared using a similar protocol to procedure 1.9 using cis-2-benzyl-3a-methyl-6-(trifluoromethyl)-3,3a,5,9b-tetrahydroisochromeno[3,4-c]pyrrol-1(2H)-one (60 mg) to afford cis-2-benzyl-3a-methyl-6-(trifluoromethyl)-1,2,3,3a,5,9b-hexahydroisochromeno[3,4-c]pyrrole (49.4% yield), EI-MS: m/z=347.9 [M+H]⁺.

### 7.5 Preparation of trans-2-benzyl-3a-methyl-6-(trifluoromethyl)-1,2,3,3a,5,9b-hexahydroisochromeno[3,4-c]pyrrole

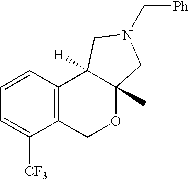

The title compound was prepared using a similar protocol to procedure 1.9 using trans-2-benzyl-3a-methyl-6-(trifluoromethyl)-3,3a,5,9b-tetrahydroisochromeno[3,4-c]pyrrol-1(2H)-one (60 mg) to afford trans-2-benzyl-3a-methyl-6-(trifluoromethyl)-1,2,3,3a,5,9b-hexahydroisochromeno[3,4-c]pyrrole (30% yield), EI-MS: m/z=348.1 [M+H]⁺.

### EXAMPLE 8

### cis-3a-Methyl-6-(trifluoromethyl)-1,2,3,3a,5,9b-hexahydroisochromeno[3,4-c]pyrrole hydrochloride

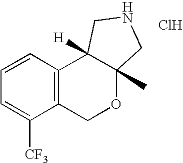

Similar reaction conditions to the protocol used in the procedure of Example 2 were repeated followed by treatment with HCl for cis-2-benzyl-3a-methyl-6-(trifluoromethyl)-1,2,3,3a,5,9b-hexahydroisochromeno[3,4-c]pyrrole (0.082 mmol, 28.5 mg) to afford cis-3a-methyl-6-(trifluoromethyl)-1,2,3,3a,5,9b-hexahydroisochromeno[3,4-c]pyrrole hydrochloride (4.98% yield), EI-MS: m/z=258.5 [M+H]⁺.

### EXAMPLE 9

### trans-3a-Methyl-6-(trifluoromethyl)-1,2,3,3a,5,9b-hexahydroisochromeno[3,4-c]pyrrole hydrochloride

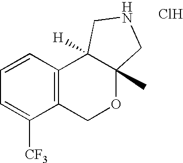

Similar reaction conditions to the protocol used in the procedure of Example 2 were repeated followed by treatment with HCl for trans-2-benzyl-3a-methyl-6-(trifluoromethyl)-1,2,3,3a,5,9b-hexahydroisochromeno[3,4-c]pyrrole (0.082 mmol, 17 mg) to afford trans-3a-methyl-6-(trifluoromethyl)-1,2,3,3a,5,9b-hexahydroisochromeno[3,4-c]pyrrole hydrochloride (9% yield), EI-MS: m/z=258.5 [M+H]⁺.

### EXAMPLE 10

### cis-8-Bromo-3a-methyl-6-(trifluoromethyl)-1,2,3,3a,5,9b-hexahydroisochromeno[3,4-c]pyrrole

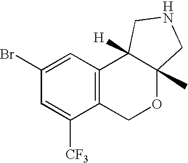

N-Bromosuccinimide (0.834 mmol, 148 mg) was added to a solution of cis-3a-methyl-6-(trifluoromethyl)-1,2,3,3a,5,9b-hexahydroisochromeno[3,4-c]pyrrole (0.758 mmol, 195 mg) in conc degassed (N₂) H₂SO₄ (1.8 ml). The reaction vessel was covered in tinfoil and the mixture was stirred overnight (16 h) then poured onto ice. The mixture was basified with 4M NaOH. The product was extracted with ethyl acetate (×3), dried (Na₂SO₄) and concentrated to afford cis-8-bromo-3a-methyl-6-(trifluoromethyl)-1,2,3,3a,5,9b-hexahydroisochromeno[3,4-c]pyrrole (92% yield), EI-MS: m/z=336.1 and 340.1 [M+H]⁺.

### EXAMPLE 11

### trans-7,10-Dichloro-2,3,4,4a,6,10b-hexahydro-1H-isochromeno[4,3-c]pyridine

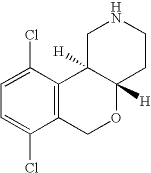

A mixture of trans-10-bromo-7-chloro-2,3,4,4a,6,10b-hexahydro-1H-isochromeno[4,3-c]pyridine (0.075 mmol, 22.7 mg) and nickel (II) chloride (0.150 mmol, 19.84 mg) in NMP (1 ml) was heated in a microwave at 210° C. for 0.5 h. Water was added and the resulting mixture was extracted with DCM, then passed through SCX column (1 g) to give a crude brown solid that was purified by prep-LCMS (basic) to give trans-7,10-dichloro-2,3,4,4a,6,10b-hexahydro-1H-isochromeno[4,3-c]pyridine (5 mg, 29%) as a white solid, EI-MS: m/z=258.0, 260.0 [M+H]⁺.

### Preparation of cis-tert-Butyl 8-bromo-3a-methyl-6-(trifluoromethyl)-1,3,3a,9b-tetrahydro isochromeno[3,4-c]pyrrole-2(5H)-carboxylate

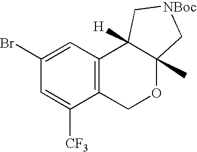

Di-tert-butyl dicarbonate (0.769 mmol, 0.168 g) was added to a suspension of cis-8-bromo-3a-methyl-6-(trifluoromethyl)-1,2,3,3a,5,9b-hexahydroisochromeno[3,4-c]pyrrole (0.699 mmol, 0.235 g) and sodium hydrogen carbonate (352 mg) in methanol (5.98 ml). Following addition, the reaction was sonicated at ambient temperature for 2.5 h during which time the temperature reached 40° C. The solvent was removed under reduced pressure and the crude material was partitioned between ethyl acetate and water. The organic phase was dried (Na₂SO₄) and evaporated to dryness under reduced pressure. Flash chromatography using ethyl acetate in heptane (10, 15 & 20%) afforded cis-tert-butyl 8-bromo-3a-methyl-6-(trifluoromethyl)-1,3,3a,9b-tetrahydroisochromeno[3,4-c]pyrrole-2(5H)-carboxylate (46% yield).

### EXAMPLE 12

### cis-8-Methoxy-3a-methyl-6-(trifluoromethyl)-1,2,3,3a,5,9b-hexahydro isochromeno[3,4-c]pyrrole hydrochloride

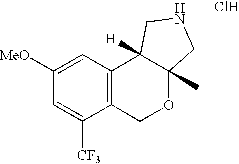

A mixture of cis-tert-butyl 8-bromo-3a-methyl-6-(trifluoromethyl)-1,3,3a,9b-tetrahydroisochromeno[3,4-c]pyrrole-2(5H)-carboxylate (0.092 mmol, 40 mg), sodium methoxide (25 wt % in MeOH, 9.17 mmol, 2.097 mL, 1982 mg) and copper(I) bromide (9.17 μmol, 1.315 mg) in DMF (1 ml) was irradiated in a microwave vial at 120° C. for 10 minutes. The mixture was partitioned between ethyl acetate and 2M NaOH. The organic phase was dried (Na₂SO₄) and concentrated to give crude residue. The residue was purified by flash chromatography using ethyl acetate in heptane (0 to 20%) as the eluent to afford a colourless residue that was dissolved in dioxane (1 ml) then methanol (0.5 ml) and HCl (5 N, 0.5 ml) were added. The mixture was heated at 100° C. for 1 hour and then concentrated by nitrogen blow down. Purification by basic prep-HPLC afforded pure product which was concentrated and converted to hydrochloride salt to give cis-8-methoxy-3a-methyl-6-(trifluoromethyl)-1,2,3,3a,5,9b-hexahydroisochromeno[3,4-c]pyrrole hydrochloride (7% yield), EI-MS: m/z=288.0 [M+H]⁺.

### EXAMPLE 13

### cis-3a,8-Dimethyl-6-(trifluoromethyl)-1,2,3,3a,5,9b-hexahydroisochromeno[3,4-c]pyrrole hydrochloride

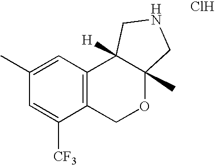

A mixture of cis-tert-butyl 8-bromo-3a-methyl-6-(trifluoromethyl)-1,3,3a,9b-tetrahydroisochromeno[3,4-c]pyrrole-2(5H)-carboxylate (0.092 mmol, 40 mg), trimethylboroxine (0.183 mmol, 0.026 ml, 23.02 mg), tetrakis(triphenylphosphine)palladium (0) (9.17 μmol, 10.60 mg) and potassium carbonate (0.183 mmol, 25.3 mg) in degassed dioxane (2 ml) were subjected to microwave irradiation at 120° C. for 20 minutes. The mixture was partitioned between ethyl acetate and water, dried (MgSO₄) and concentrated under reduced pressure to afford a crude residue. Purification by flash chromatography using ethyl acetate in heptane (10 to 20%) as the eluent afforded Boc-protected intermediate. HCl (5N, 2.86 mmol, 0.571 ml) was added to a solution of the Boc-protected intermediate (15 mg) in dioxane (1 ml) and methanol (0.5 ml). The mixture was stirred at 100° C. for 0.5 h, concentrated under reduced pressure then purified by SCX chromatography followed by basic prep-HPLC. The product was converted to the HCl salt to give cis-3a,8-dimethyl-6-(trifluoromethyl)-1,2,3,3a,5,9b-hexahydroisochromeno[3,4-c]pyrrole hydrochloride (4% yield), EI-MS: m/z=272.4 [M+H]⁺.

### EXAMPLE 14

### cis-8-Ethyl-3a-methyl-6-(trifluoromethyl)-1,2,3,3a,5,9b-hexahydroisochromeno[3,4-c]pyrrole hydrochloride

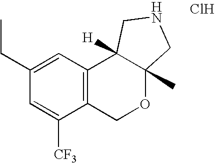

Tetrakis(triphenylphosphine)palladium (0) (9.17 μmol, 10.60 mg) was added in one portion to a mixture of cis-tert-butyl 8-bromo-3a-methyl-6-(trifluoromethyl)-1,3,3a,9b-tetrahydroisochromeno[3,4-c]pyrrole-2(5H)-carboxylate (0.092 mmol, 40 mg), 2,4,6-trivinylcyclotriboroxane pyridine complex (0.092 mmol, 22.07 mg), potassium carbonate (0.183 mmol, 25.3 mg), degassed 1,2-dimethoxyethane (0.860 ml) and water (0.287 ml). The mixture was heated at 100° C. for 1.5 hours and then allowed to cool to room temperature, diluted with brine and extracted with ethyl acetate (×3). The combined ethyl acetate extracts were dried (MgSO₄), filtered and concentrated under reduced pressure to afford the crude residue. Purification by flash chromatography, eluting with ethyl acetate in heptane (5 to 30%) afforded the intermediate alkene (26 mg). To the intermediate alkene (26 mg) and 10% palladium on carbon (˜4 mg), under an inert atmosphere, was added ethanol (5 ml). The reaction mixture was stirred under an atmosphere of hydrogen (balloon) for 4 hours. The spent catalyst was removed by filtering through dicalite and washed with methanol. Concentration of the filtrate afforded the Boc-protected ethyl derivative (24 mg). 5N HCl (2.86 mmol, 0.571 ml) was added to a solution of the Boc-protected ethyl derivative (24 mg) in dioxane (1 ml) and methanol (0.5 ml). The mixture was stirred at 100° C. for 0.5 h then the solvent was removed and the residue purified by SCX, followed by basic prep-HPLC to afford the desired product which was converted to the HCl salt, using HCl in ether, to afford cis-8-ethyl-3a-methyl-6-(trifluoromethyl)-1,2,3,3a,5,9b-hexahydroisochromeno[3,4-c]pyrrole hydrochloride (30% yield), EI-MS: m/z=286.0 [M+H]⁺

### EXAMPLE 15

### cis-8-Chloro-3a-methyl-6-(trifluoromethyl)-1,2,3,3a,5,9b-hexahydroisochromeno[3,4-c]pyrrole hydrochloride

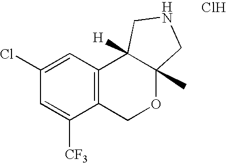

A mixture of cis-tert-butyl 8-bromo-3a-methyl-6-(trifluoromethyl)-1,3,3a,9b-tetrahydroisochromeno[3,4-c]pyrrole-2(5H)-carboxylate (0.092 mmol, 40 mg) and nickel (II) chloride (0.367 mmol, 47.5 mg) in N-methyl-2-pyrrolidinone (2 mL) was subjected to microwave irradiation at 210° C. for 30 minutes. The reaction mixture was partitioned between ethyl acetate and 2M NaOH. The organic phase was dried (Na₂SO₄) and concentrated under reduced pressure to give a crude residue. Analysis by LC-MS indicated that the Boc-protecting group had been removed during the synthesis. Purification by basic prep-HPLC afforded the desired product which was readily converted to the HCl salt (using HCl in ether) to afford cis-8-chloro-3a-methyl-6-(trifluoromethyl)-1,2,3,3a,5,9b-hexahydroisochromeno[3,4-c]pyrrole hydrochloride (28% yield), EI-MS: m/z=286.0 [M+H]⁺.

### EXAMPLE 16

### trans-8-Benzyloxy-1,2,3,3a,5,9b-hexahydroisochromeno[3,4-c]pyrrole

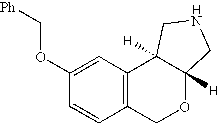

### 16.1 Preparation of N-benzyl-5-(benzyloxy)-N-(2-hydroxyethyl)-1,2-dihydro cyclobutabenzene-1-carboxamide

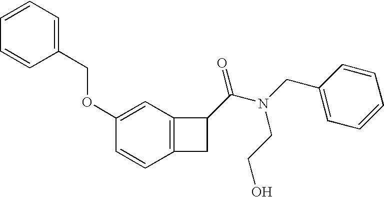

A solution of 5-(benzyloxy)-1,2-dihydrocyclobutabenzene-1-carboxylic acid (4 g, 15.73 mmol, prepared as described by Loozen et al, Journal of the Royal Netherlands Chemical Society, 101/9, 1982) in thionyl chloride (361 mmol, 26.4 ml, 43 g) was stirred for 1 h at ambient temperature (several drops of DMF were added to promote the reaction) and then the reaction was stirred at reflux for 0.5 h. Excess thionyl chloride was removed using a rotary evaporator. The acid chloride was dissolved in dichloromethane (26 ml) and added dropwise to a cooled solution (−50° C.) of 2-(benzylamino)ethanol (23.61 mmol, 3.57 g) and triethylamine (37.0 mmol, 5.19 ml, 3.74 g) in dichloromethane (30.00 ml). The mixture was stirred at −50° C. for 1 h and then at ambient temperature for a further 1 h. Water was then added and the layers separated. The organic layer was washed with 0.5 M aqueous hydrochloride (×2), dried (MgSO₄) and concentrated to give a residue. The residue was flash chromatographed using ethyl acetate in heptane (10% to 50%) as the eluent to give N-benzyl-5-(benzyloxy)-N-(2-hydroxyethyl)-1,2-dihydrocyclobutabenzene-1-carboxamide (89% yield), EI-MS: m/z=388.4 [M+H]⁺.

### 16.2 Preparation of N-benzyl-5-(benzyloxy)-N-(2-oxoethyl)-1,2-dihydro cyclobutabenzene-1-carboxamide

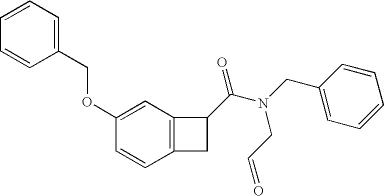

Dess-Martin periodinane (21.10 mmol, 8.95 g) in dichloromethane (260 ml) was added to a solution of N-benzyl-5-(benzyloxy)-N-(2-hydroxyethyl)-1,2-dihydrocyclobutabenzene-1-carboxamide (14.07 mmol, 5.45 g) in dichloromethane (132 ml). The mixture was stirred at room temperature for 2 h. Saturated aqueous NaHCO₃ (150 ml) was added and the mixture was stirred for 30 minutes. The mixture was diluted with additional dichloromethane (300 ml) and water (200 ml). The organic phase was separated, washed with water, dried (MgSO₄) and concentrated under reduced pressure to afford a crude residue that was flash chromatographed using ethyl acetate in toluene (5-10%) as the eluent to afford N-benzyl-5-(benzyloxy)-N-(2-oxoethyl)-1,2-dihydrocyclobutabenzene-1-carboxamide (45% yield), EI-MS: m/z=386.4 [M+H]⁺.

### 16.3 Preparation of trans-2-benzyl-8-(benzyloxy)-3,3a,5,9b-tetrahydro isochromeno[3,4-c]pyrrol-1(2H)-one & cis-2-benzyl-8-(benzyloxy)-3,3a,5,9b-tetrahydroisochromeno[3,4-c]pyrrol-1(2H)-one

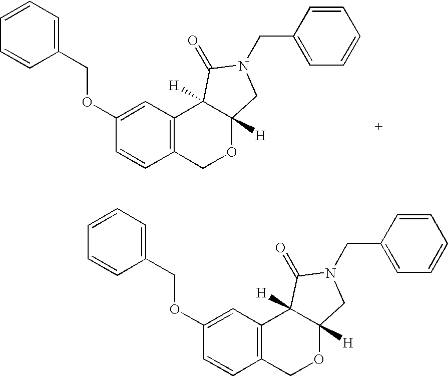

A solution of N-benzyl-5-(benzyloxy)-N-(2-oxoethyl)-1,2-dihydrocyclobutabenzene-1-carboxamide (18.76 mmol, 7.23 g) in bromobenzene (188 ml) was heated at reflux for 16 hours. The solvent was removed in vacuo to afford a crude residue that was purified by flash chromatography using toluene followed by ethyl acetate in toluene (2%) as the eluent to afford trans-2-benzyl-8-(benzyloxy)-3,3a,5,9b-tetrahydroisochromeno[3,4-c]pyrrol-1(2H)-one (46% yield), EI-MS: m/z=386.5 [M+H]⁺, followed by cis-2-benzyl-8-(benzyloxy)-3,3a,5,9b-tetrahydroisochromeno[3,4-c]pyrrol-1(2H)-one (30% yield), EI-MS: m/z=386.5 [M+H]⁺.

### 16.4 Preparation of trans-2-benzyl-8-(benzyloxy)-1,2,3,3a,5,9b-hexahydro isochromeno[3,4-c]pyrrole

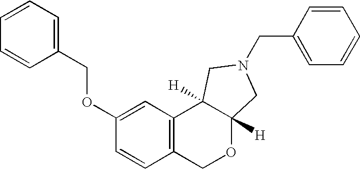

Borane-dimethyl sulfide complex (122 mmol, 11.69 ml, 9.24 g) was added to a solution of trans-2-benzyl-8-(benzyloxy)-3,3a,5,9b-tetrahydroisochromeno[3,4-c]pyrrol-1(2H)-one (16.21 mmol, 6.25 g) in tetrahydrofuran (162 ml). The mixture stirred at reflux under nitrogen for 5 h then cooled to 5° C. and 5 N aqueous HCl (50 ml) was carefully added. The mixture stirred at reflux for an additional 1.5 h and then left to stand overnight (16 h). After addition of excess saturated aqueous NaHCO₃ (1000 ml), the mixture was extracted with EtOAc (750 ml, ×3), the organics combined, dried (MgSO₄) and concentrated under reduced pressure to give a residue. The crude residue was flash chromatographed using toluene then EtOAc in toluene (5%, 10%, 15% and 20%) to afford trans-2-benzyl-8-(benzyloxy)-1,2,3,3a,5,9b-hexahydroisochromeno[3,4-c]pyrrole (56% yield), EI-MS: m/z=372.1 [M+H]⁺.

### 16.5 Preparation of trans-tert-butyl 8-hydroxy-1,3,3a,9b-tetrahydroisochromeno[3,4-c]pyrrole-2(5H)-carboxylate

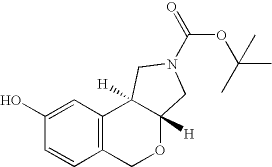

A suspension of trans-2-benzyl-8-(benzyloxy)-1,2,3,3a,5,9b-hexahydroisochromeno[3,4-c]pyrrole (8.08 mmol, 3 g) and 10% Pd/C (481 mg) in acetic acid (70.0 mL) was stirred at ambient temperature and 1.5 bar pressure under a hydrogen atmosphere for 20 hours. The mixture was filtered through Dicalite® to remove catalyst and the filtrate concentrated under reduced pressure. The above conditions were repeated with fresh catalyst, stirring under an atmosphere of hydrogen for 40 hours. The mixture was filtered through Dicalite® to remove catalyst and the filtrate concentrated under reduced pressure to afford a crude residue. The residue was loaded onto a pre-acidified SCX column and eluted with 2M NH₃ in methanol to afford the free base. The product was concentrated then treated with triethylamine (24.23 mmol, 3.40 mL, 2.452 g), DMAP (2.423 mmol, 0.296 g), dichloromethane (40 mL) and di-tert-butyl dicarbonate (16.15 mmol, 3.53 g). The mixture was stirred for 4 hours then the solvent was removed under reduced pressure and the residue obtained partitioned between ethyl acetate and 0.5M citric acid. The organic layer was separated and the aqueous extracted with further ethyl acetate. The combined organics were dried (MgSO₄) and concentrated under reduced pressure to afford crude di-BOC protected product. This was purified by flash chromatography using ethyl acetate in toluene (0% to 30%) to afford 2.14 g of the di-BOC protected product. The product was dissolved in methanol (225 mL) and KOH (36.3 mmol, 2.039 g) was added. The mixture was heated at reflux for 1 hour then concentrated under reduced pressure. The residue was partitioned between water and ethyl acetate. The organic layer was dried (MgSO₄) and concentrated under reduced pressure to afford trans-tert-butyl 8-hydroxy-1,3,3a,9b-tetrahydroisochromeno[3,4-c]pyrrole-2(5H)-carboxylate (56% yield), EI-MS: m/z=292.3 [M+H]⁺.

### 16.6 Preparation of trans-8-benzyloxy-1,2,3,3a,5,9b-hexahydroisochromeno[3,4-c]pyrrole

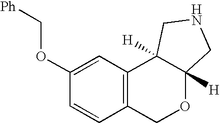

To a solution of trans-tert-butyl 8-hydroxy-1,3,3a,9b-tetrahydroisochromeno[3,4-c]pyrrole-2(5H)-carboxylate (0.285 mmol, 83 mg) in acetone (7.5 ml) was added potassium carbonate (1.424 mmol, 197 mg) followed by benzyl bromide (0.570 mmol, 0.068 ml, 97 mg). The resultant suspension was heated at 60° C. for 16 h and then diethylamine (0.5 ml) was added and the mixture stirred at 60° C. for 3 h before filtering and concentrated in vacuo. The residue was partitioned between 0.5 M citric acid solution and dichloromethane. The chlorinated phase (10 ml) was collected through a hydrophobic frit and trifluoroacetic acid (1 ml) added. After stirring for 2 h the solution was concentrated in vacuo to give a residue that was converted to the free base using ion exchange chromatography (SCX) to afford trans-8-benzyloxy-1,2,3,3a,5,9b-hexahydroisochromeno[3,4-c]pyrrole (100% yield), EI-MS: m/z=282.5 [M+H]⁺.

### EXAMPLE 17

### cis-8-Methoxy-1,2,3,3a,5,9b-hexahydroisochromeno[3,4-c]pyrrole hydrochloride

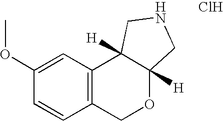

### 17.1 Preparation of cis-2-benzyl-8-(benzyloxy)-1,2,3,3a,5,9b-hexahydroisochromeno[3,4-c]pyrrole

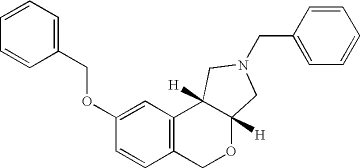

Similar reaction conditions to the protocol described in Example 16.4 were repeated for cis-2-benzyl-8-(benzyloxy)-3,3a,5,9b-tetrahydroisochromeno[3,4-c]pyrrol-1(2H)-one (13.36 mmol, 5.15 g) to afford cis-2-benzyl-8-(benzyloxy)-1,2,3,3a,5,9b-hexahydroisochromeno[3,4-c]pyrrole (64% yield), EI-MS: m/z=372.1 [M+H]⁺.

### 17.2 Preparation of cis-tert-butyl 8-hydroxy-1,3,3a,9b-tetrahydroisochromeno[3,4-c]pyrrole-2(5H)-carboxylate

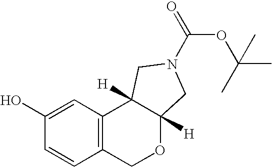

Similar reaction conditions to that in procedure 16.5 were repeated for cis-2-benzyl-8-(benzyloxy)-1,2,3,3a,5,9b-hexahydroisochromeno[3,4-c]pyrrole (8.56 mmol, 3.18 g) to afford cis-tert-butyl 8-hydroxy-1,3,3a,9b-tetrahydroisochromeno[3,4-c]pyrrole-2(5H)-carboxylate (66% yield), EI-MS: m/z=292.3 [M+H]⁺.

### 17.3 Preparation of cis-8-methoxy-1,2,3,3a,5,9b-hexahydroisochromeno[3,4-c]pyrrole hydrochloride

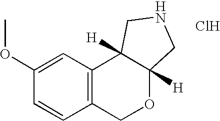

Iodomethane (0.178 mmol) was added to a solution of cis-tert-butyl 8-hydroxy-1,3,3a,9b-tetrahydroisochromeno[3,4-c]pyrrole-2(5H)-carboxylate (26 mg, 0.089 mmol) and K₂CO₃ (62 mg, 0.446 mmol) in acetone (2.5 ml) and the mixture heated at 60° C. The mixture was stirred for 24 h. Diethylamine (0.5 ml) was added and the mixture was stirred at 60° C. for 3 h then the mixture was filtered and concentrated in vacuo. The residue was partitioned between 1 M HCl (2 ml) and dichloromethane (3 ml). TFA (1 ml) was added to the organic phase which was stirred for 3 h then concentrated in vacuo to give a residue that was loaded onto a pre-acidified SCX column using MeOH and eluted with 2M NH₃ in MeOH. The product was concentrated in vacuo and purified under basic prep-HPLC conditions to give the free base which was converted to the HCl salt by treatment with HCl in ether followed by concentration in vacuo to afford cis-8-methoxy-1,2,3,3a,5,9b-hexahydroisochromeno[3,4-c]pyrrole hydrochloride (36% yield), EI-MS: m/z=206.3 [M+H]⁺.

### EXAMPLE 18

### cis-8-Ethoxy-1,2,3,3a,5,9b-hexahydroisochromeno[3,4-c]pyrrole hydrochloride

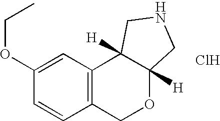

Similar reaction conditions for procedure 17.3 were repeated using iodoethane to afford cis-8-ethoxy-1,2,3,3a,5,9b-hexahydroisochromeno[3,4-c]pyrrole hydrochloride (36% yield), EI-MS: m/z=220.4 [M+H]⁺.

### EXAMPLE 19

### cis-8-(2-Fluorobenzyloxy)-1,2,3,3a,5,9b-hexahydroisochromeno[3,4-c]pyrrole

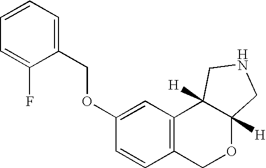

Similar reaction conditions to that in procedure 16.6 were repeated using 2-fluorobenzyl bromide to afford cis-8-(2-fluorobenzyloxy)-1,2,3,3a,5,9b-hexahydroisochromeno[3,4-c]pyrrole (40% yield), EI-MS: m/z=300.5 [M+H]⁺.

### EXAMPLE 20

### trans-7,9-Dibromo-8-methoxy-1,2,3,3a,5,9b-hexahydroisochromeno[3,4-c]pyrrole hydrochloride

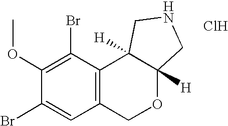

N-Bromosuccinimide (0.275 mmol, 48.9 mg) was added to a solution of trans-tert-butyl 8-hydroxy-1,3,3a,9b-tetrahydroisochromeno[3,4-c]pyrrole-2(5H)-carboxylate (0.137 mmol, 40 mg) in tetrahydrofuran (0.686 ml). The mixture was stirred at ambient temperature for 1 hour. The mixture was concentrated under reduced pressure and the resultant residue diluted with a saturated aqueous solution of sodium bicarbonate (10 ml) and extracted with dichloromethane (3×10 ml). The combined organic extracts were dried over sodium sulphate, filtered and evaporated to give a crude oil that was purified by flash chromatography using ethyl acetate in heptane (5% to 30%) as the eluent to afford trans-tert-butyl 7,9-dibromo-8-hydroxy-1,3,3a,9b-tetrahydroisochromeno[3,4-c]pyrrole-2(5H)-carboxylate (62% yield, 38 mg), MS m/z=450.0, [MH+]. Iodomethane (0.169 mmol, 10.53 μl, 24.02 mg) was added to trans-tert-butyl 7,9-dibromo-8-hydroxy-1,3,3a,9b-tetrahydroisochromeno[3,4-c]pyrrole-2(5H)-carboxylate (0.085 mmol, 38 mg) and potassium carbonate (0.423 mmol, 58.5 mg) in acetone (2 ml). The resultant suspension was heated at 60° C. for 2 h and then concentrated under reduced pressure. The residue was partitioned between 0.5 M citric acid and dichloromethane. The chlorinated phase was collected through a hydrophobic frit and TFA (3.37 mmol, 250 μl) then added. After stirring for 2 h the solution was concentrated under reduced pressure to give a residue that was converted to the free base using ion exchange chromatography (SCX). The free base was purified using basic prep-HPLC then converted to the HCl salt using HCl in diethyl ether and concentrated in vacuo to afford of trans-7,9-dibromo-8-methoxy-1,2,3,3a,5,9b-hexahydroisochromeno[3,4-c]pyrrole hydrochloride (60% yield, 20.3 mg), EI-MS: m/z=364.1 [M+H]⁺.

### EXAMPLE 21

### cis-7,9-Dibromo-8-methoxy-1,2,3,3a,5,9b-hexahydroisochromeno[3,4-c]pyrrole hydrochloride

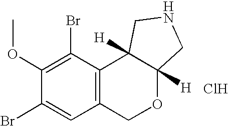

A similar reaction protocol to the procedure of Example 20 was repeated for cis-tert-butyl 7,9-dibromo-8-hydroxy-1,3,3a,9b-tetrahydroisochromeno[3,4-c]pyrrole-2(5H)-carboxylate (0.076 mmol, 34.0 mg) to afford cis-7,9-dibromo-8-methoxy-1,2,3,3a,5,9b-hexahydroisochromeno[3,4-c]pyrrole hydrochloride (53% yield), EI-MS: m/z=364.3 [M+H]⁺.

### EXAMPLE 22

### cis-9-Chloro-8-methoxy-1,2,3,3a,5,9b-hexahydroisochromeno[3,4-c]pyrrole hydrochloride

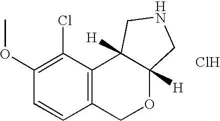

### 22.1 Preparation of cis-tert-butyl 7,9-dichloro-8-hydroxy-1,3,3a,9b-tetrahydroisochromeno[3,4-c]pyrrole-2(5H)-carboxylate & cis-tert-butyl 9-chloro-8-hydroxy-1,3,3a,9b-tetrahydroisochromeno[3,4-c]pyrrole-2(5H)-carboxylate

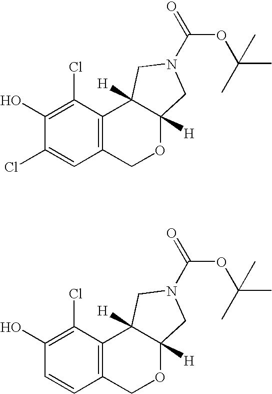

cis-tert-Butyl 8-hydroxy-1,3,3a,9b-tetrahydroisochromeno[3,4-c]pyrrole-2(5H)-carboxylate (0.309 mmol, 89.9 mg) was dissolved in tetrahydrofuran (1498 μl). The resulting solution was stirred at 0° C. and N-chlorosuccinimide (0.617 mmol, 82 mg) was added. After stirring for 30 mins the ice bath was removed and the reaction mixture was stirred at ambient temperature for 17 hours. The mixture was evaporated under reduced pressure and the residue was mixed with a saturated aqueous solution of sodium bicarbonate (50 ml) and extracted with ethyl acetate (3×50 ml). The combined extracts were dried over sodium sulphate, filtered and evaporated to give a residue. Flash chromatography of the residue using dichloromethane followed by ethyl acetate in dichloromethane (25%) as the eluent afforded cis-tert-butyl 7,9-dichloro-8-hydroxy-1,3,3a,9b-tetrahydroisochromeno[3,4-c]pyrrole-2(5H)-carboxylate (64% yield), followed by cis-tert-butyl 9-chloro-8-hydroxy-1,3,3a,9b-tetrahydroisochromeno[3,4-c]pyrrole-2(5H)-carboxylate (40% yield).

### 22.2 cis-9-Chloro-8-methoxy-1,2,3,3a,5,9b-hexahydroisochromeno[3,4-c]pyrrole hydrochloride

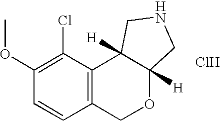

Similar reaction conditions for the methylation and deprotection in the procedure of Example 20 were repeated for cis-tert-butyl 9-chloro-8-hydroxy-1,3,3a,9b-tetrahydroisochromeno[3,4-c]pyrrole-2(5H)-carboxylate (0.105 mmol, 34.1 mg) to afford cis-9-chloro-8-methoxy-1,2,3,3a,5,9b-hexahydroisochromeno[3,4-c]pyrrole hydrochloride (11.5 mg), EI-MS: m/z=240.1 [M+H]⁺.

### EXAMPLE 23

### cis-7,9-Dichloro-8-methoxy-1,2,3,3a,5,9b-hexahydroisochromeno[3,4-c]pyrrole hydrochloride

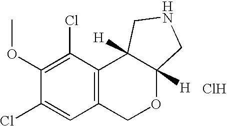

Similar reaction conditions for the methylation and deprotection in the procedure of Example 20 were repeated for cis-tert-butyl 7,9-dichloro-8-hydroxy-1,3,3a,9b-tetrahydroisochromeno[3,4-c]pyrrole-2(5H)-carboxylate (0.073 mmol, 26.2 mg) to afford cis-7,9-dichloro-8-methoxy-1,2,3,3a,5,9b-hexahydroisochromeno[3,4-c]pyrrole hydrochloride (77% yield), EI-MS: m/z=274.0 [M+H]⁺.

### EXAMPLE 24

### cis-8-Methyl-1,2,3,3a,5,9b-hexahydroisochromeno[3,4-c]pyrrole hydrochloride

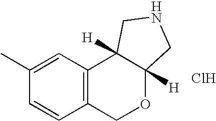

### 24.1 Preparation of cis-tert-butyl 8-(trifluoromethylsulfonyloxy)-1,3,3a,9b-tetrahydroisochromeno[3,4-c]pyrrole-2(5H)-carboxylate

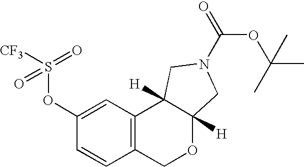

To a solution of cis-tert-butyl 8-hydroxy-1,3,3a,9b-tetrahydroisochromeno[3,4-c]pyrrole-2(5H)-carboxylate (0.172 mmol, 50 mg) in tetrahydrofuran (1.7 mL) was added sodium hydride (0.206 mmol, 8.24 mg) followed by N-phenyltrifluoromethanesulfonamide (0.172 mmol, 61.3 mg). The reaction was stirred for 16 h then the THF was removed under reduced pressure and the residue partitioned between dichloromethane and NaHCO₃ (aq). The organic layer was collected via a hydrophobic frit and concentrated under reduced pressure to afford cis-tert-butyl 8-(trifluoromethylsulfonyloxy)-1,3,3a,9b-tetrahydroisochromeno[3,4-c]pyrrole-2(5H)-carboxylate (99% yield).

### 24.2 cis-8-Methyl-1,2,3,3a,5,9b-hexahydroisochromeno[3,4-c]pyrrole hydrochloride

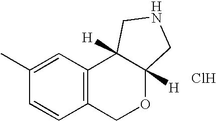

A mixture of cis-tert-butyl 8-(trifluoromethylsulfonyloxy)-1,3,3a,9b-tetrahydroisochromeno[3,4-c]pyrrole-2(5H)-carboxylate (0.059 mmol, 25 mg), trimethoxyboroxine (0.118 mmol, 0.017 mL, 14.82 mg), tetrakis(triphenylphosphine)palladium (0) (0.012 mmol, 13.65 mg) and potassium carbonate (0.118 mmol, 16.32 mg) in dioxane (1 mL) was stirred at 100° C. for 18 hours. Solvent was removed in vacuo and the crude residue partitioned between EtOAc and water, and the layers separated. The organic phase was washed with brine, dried (Na₂SO₄) and concentrated under reduced pressure to afford cis-tert-butyl 8-methyl-1,3,3a,9b-tetrahydroisochromeno[3,4-c]pyrrole-2(5H)-carboxylate (0.059 mmol, 17 mg) which was treated with dichloromethane (2 mL) and trifluoroacetic acid (0.5 mL). After stirring for 1 h the mixture was concentrated in vacuo to give a residue which was loaded onto a pre-acidified SCX column and eluted with 2M NH₃ in MeOH. The basic filtrate was concentrated in vacuo and the residue purified by basic prep-LCMS. The desired fraction were concentrated in vacuo and treated with HCl in ether then concentrated in vacuo to afford cis-8-methyl-1,2,3,3a,5,9b-hexahydroisochromeno[3,4-c]pyrrole hydrochloride (6.03% yield), EI-MS: m/z=190.6 [M+H]⁺.

### EXAMPLE 25

### cis-7-Methoxy-1,2,3,3a,5,9b-hexahydroisochromeno[3,4-c]pyrrole

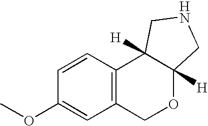

### 25.1 Preparation of 5-(benzyloxy)-2-bromobenzaldehyde

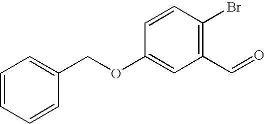

Bromine (471 mmol, 24.14 ml, 75 g) was added slowly to a stirred solution of 3-(benzyloxy)benzaldehyde (236 mmol, 50 g) in acetic acid (200 ml) with sodium acetate (353 mmol, 29.0 g) at 0° C. A calcium chloride guard tube was fitted and the resultant mixture was stirred, in the dark at room temperature, for 16 hours. The mixture was diluted with dichloromethane and washed with aq Na₂S₂O₃, aq K₂CO₃ and finally water. The organic layer was dried (Na₂SO₄) and concentrated under reduced pressure to give 5-(benzyloxy)-2-bromobenzaldehyde (74 g).

### 25.2 Preparation of 3-(5-(benzyloxy)-2-bromophenyl)-2-cyanoacrylic acid

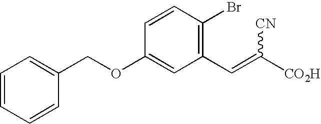

A stirred mixture of 5-(benzyloxy)-2-bromobenzaldehyde (254 mmol, 74 g), cyanoacetic acid (254 mmol, 21.62 g), ammonium acetate (50.8 mmol, 3.92 g) and pyridine (36 ml) in toluene (196 ml) was refluxed using Dean Stark apparatus until one molar equivalent of water was collected. The reaction mixture was allowed to cool and then concentrated to form a residue that was then stirred with 10% aqueous HCl. The product was extracted with ethyl acetate, dried (Na₂SO₄) and concentrated to afford a crude solid on standing. Recrystallisation from toluene gave 3-(5-(benzyloxy)-2-bromophenyl)-2-cyanoacrylic acid (27.9% yield)

### 25.3 Preparation of 3-(5-(benzyloxy)-2-bromophenyl)-2-cyanopropanoic acid

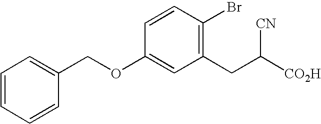

Sodium borohydride (259 mmol, 9.78 g) was added, in portions over a period of 45 minutes, to a stirred suspension of 3-(5-(benzyloxy)-2-bromophenyl)-2-cyanoacrylic acid (72.6 mmol, 26 g) in aqueous saturated NaHCO₃ (83 mL) and methanol (202 ml) at 15° C. The reaction mixture was allowed to warm to room temperature and was stirred for 1 hour before removing the methanol under reduced pressure. The resultant mixture was diluted with water and washed with ether. The aqueous layer was acidified and the desired product was extracted with ether. The combined organics were dried (Na₂SO₄) and evaporated under reduced pressure to afford 3-(5-(benzyloxy)-2-bromophenyl)-2-cyanopropanoic acid (97% yield, 25.36 g)

### 25.4 Preparation of 3-(5-(benzyloxy)-2-bromophenyl)propanenitrile

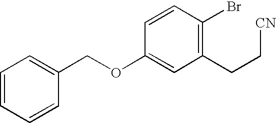

3-(5-(Benzyloxy)-2-bromophenyl)-2-cyanopropanoic acid (70.4 mmol, 25.36 g) was dissolved in DMA (35.2 ml) and heated for an hour and a half at 150° C. After cooling, the reaction mixture was poured into water and extracted with ether. The organic layer was washed with a saturated solution of sodium hydrogen carbonate and then brine. The organic phase was dried (Na₂SO₄) and evaporated under reduced pressure to afford 3-(5-(benzyloxy)-2-bromophenyl)propanenitrile (98% yield, 21.73 g).

### 25.5 Preparation of 4-(benzyloxy)-1,2-dihydrocyclobutabenzene-1-carbonitrile

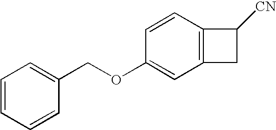

Ammonia gas was condensed into the flask from a cylinder until approximately required volume was present (˜150 mL). Commercial sodium amide (270 mmol, 10.54 g) was added to the ammonia at −78° C. and after stirring for 10 minutes 3-(5-(benzyloxy)-2-bromophenyl)propanenitrile (68.7 mmol, 21.73 g) was added over a 5 minute period. The mixture was allowed to warm such that the resultant mixture was stirred at reflux for 3 h before being neutralised with solid ammonium nitrate (298 mmol, 23.82 g) and allowed to stand overnight under a flow of nitrogen. All the ammonia evaporated and water was added to the solid residue and the products were extracted with dichloromethane (×3). The combined organics were washed with dilute hydrochloric acid (5%), followed by water. The organics were dried (Na₂SO₄) then concentrated in vacuo to afford 4-(benzyloxy)-1,2-dihydrocyclobutabenzene-1-carbonitrile (98% yield, 15.84 g)

### 25.6 Preparation of 4-(benzyloxy)-1,2-dihydrocyclobutabenzene-1-carboxylic acid

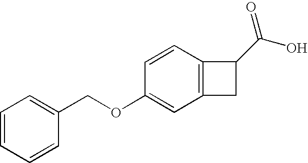

A solution of 4-(benzyloxy)-1,2-dihydrocyclobutabenzene-1-carbonitrile (67.3 mmol, 15.84 g) and KOH (337 mmol, 18.89 g) in ethanol (224 ml) and water (44.9 ml) was refluxed for 2.5 h. After evaporation of the solvent under reduced pressure, the aqueous residue was diluted with 2N NaOH (1 L) and washed with Et₂O (2×750 mL). The aqueous phase was then acidified with 5N HCl, during which a precipitate formed that was collected by filtration and dried in vacuo to afford 4-(benzyloxy)-1,2-dihydrocyclobutabenzene-1-carboxylic acid (99% yield, 16.95 g), EI-MS: m/z=253.3 [M−H]⁻.

### 25.7 Preparation of N-benzyl-4-(benzyloxy)-N-(2-hydroxyethyl)-1,2-dihydro cyclobutabenzene-1-carboxamide

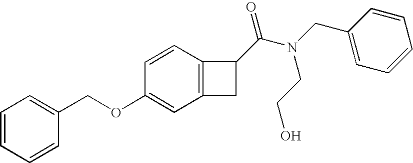

A mixture of 4-(benzyloxy)-1,2-dihydrocyclobutabenzene-1-carboxylic acid (3.93 mmol, 1 g), 2-(benzylamino)ethanol (5.90 mmol, 0.838 ml, 0.892 g), triethylamine (7.87 mmol, 1.105 ml, 0.796 g) and cyclophos (4.72 mmol, 2.81 ml in ethyl acetate) in dichloromethane (19.66 ml) was stirred at room temperature for 2 h. The reaction mixture was partitioned between dichloromethane and 2 N HCl. The aqueous layer was extracted with dichloromethane and the combined organic layers washed with water then brine, dried (Na₂SO₄) and concentrated under reduced pressure. The residue was purified by flash chromatography using ethyl acetate in heptane as the eluent (30% to 90%) to afford N-benzyl-4-(benzyloxy)-N-(2-hydroxyethyl)-1,2-dihydrocyclobutabenzene-1-carboxamide (75% yield, 1.15 g), EI-MS: m/z=388.1 [M+H]⁺.

Alternatively, a solution of 4-(benzyloxy)-1,2-dihydrocyclobutabenzene-1-carboxylic acid (7.87 mmol, 2 g) in thionyl chloride (181 mmol, 13.20 ml, 21.52 g) was stirred for 1 h at ambient temperature (several drops of DMF were added to promote the reaction) and then the reaction was refluxed for 0.5 h. Excess thionyl chloride was removed using a rotary evaporator. The acid chloride was dissolved in dichloromethane (13 ml) and added dropwise to a cooled solution (−50° C.) of 2-(benzylamino)ethanol (11.80 mmol, 1.784 g) and triethylamine (18.47 mmol, 2.60 ml, 1.869 g) in dichloromethane (15.00 ml). The mixture was stirred at −50° C. for 1 h and then at ambient temperature for a further 1 h. Water was then added and the layers separated. The organic layer was washed with 0.5 M aqueous hydrochloric acid (×2), dried (MgSO₄) and concentrated to give a residue. The crude residue was flash chromatographed using ethyl acetate in heptane (10% to 50%) as the eluent to give N-benzyl-4-(benzyloxy)-N-(2-hydroxyethyl)-1,2-dihydrocyclobutabenzene-1-carboxamide (82% yield) MS m/z=388.1 [MH+].

### 25.8 Preparation of N-benzyl-4-(benzyloxy)-N-(2-oxoethyl)-1,2-dihydro cyclobutabenzene-1-carboxamide

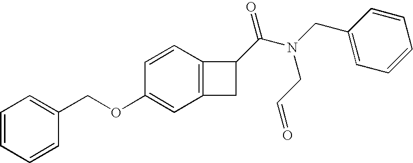

Dess-Martin periodinane (15 wt % soln in DCM, 6.45 mmol, 18 mL, 18.24 g) was added to N-benzyl-4-(benzyloxy)-N-(2-hydroxyethyl)-1,2-dihydrocyclobutabenzene-1-carboxamide (6.45 mmol, 2.5 g) in anhydrous dichloromethane (7 mL). The mixture was stirred for 2.5 h then saturated aqueous NaHCO₃ was added and the mixture was extracted with dichloromethane (×3), dried (Na₂SO₄) and concentrated in vacuo to give a residue. Flash chromatography of the residue using ethyl acetate in toluene as the eluent (5%-50%) gave N-benzyl-4-(benzyloxy)-N-(2-oxoethyl)-1,2-dihydrocyclobutabenzene-1-carboxamide (76% yield), EI-MS: m/z=386.4 [M+H]⁺.

### 25.9 Preparation of cis-2-benzyl-7-(benzyloxy)-3,3a,5,9b-tetrahydro isochromeno[3,4-c]pyrrol-1(2H)-one & trans-2-benzyl-7-(benzyloxy)-3,3a,5,9b-tetrahydroisochromeno[3,4-c]pyrrol-1(2H)-one

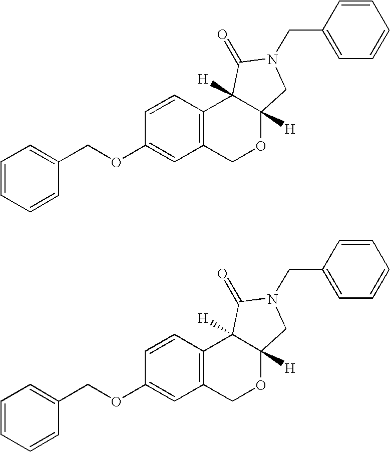

A stirred solution of N-benzyl-4-(benzyloxy)-N-(2-oxoethyl)-1,2-dihydrocyclobutabenzene-1-carboxamide (4.93 mmol, 1.9 g) in bromobenzene (590 mmol, 62 ml, 93 g) was heated at 150° C. overnight. The mixture was concentrated under reduced pressure to give a residue. Flash chromatography of the residue using ethyl acetate in toluene (2% to 10%) afforded trans-2-benzyl-7-(benzyloxy)-3,3a,5,9b-tetrahydroisochromeno[3,4-c]pyrrol-1(2H)-one (15% yield), EI-MS: m/z=386.4 [M+H]⁺, followed by cis-2-benzyl-7-(benzyloxy)-3,3a,5,9b-tetrahydroisochromeno[3,4-c]pyrrol-1(2H)-one (27% yield), EI-MS: m/z=386.3 [M+H]⁺.

### 25.10 Preparation of cis-2-benzyl-7-(benzyloxy)-1,2,3,3a,5,9b-hexahydro isochromeno[3,4-c]pyrrole

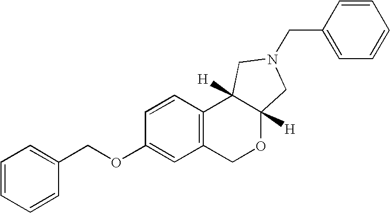

Borane-dimethyl sulfide complex (10.02 mmol, 0.968 ml, 861 mg) was added to a solution of cis-2-benzyl-7-(benzyloxy)-3,3a,5,9b-tetrahydroisochromeno[3,4-c]pyrrol-1(2H)-one (1.336 mmol, 515 mg) in tetrahydrofuran (14 ml). The resultant mixture was refluxed under nitrogen for 6 h then cooled to 5° C. and 5N aqueous HCl (4 ml) was added. The mixture was refluxed for an additional 1.5 h and then left to stand overnight (16 h). Excess saturated aqueous NaHCO₃ was added and the mixture was extracted with ethyl acetate (×3), the organics were combined, dried (MgSO₄) and concentrated under reduced pressure to give a residue. Flash chromatography of the residue using ethyl acetate in toluene as the eluent (5% to 30%) gave cis-2-benzyl-7-(benzyloxy)-1,2,3,3a,5,9b-hexahydroisochromeno[3,4-c]pyrrole (82% yield), EI-MS: m/z=372.5 [M+H]⁺.

### 25.11 Preparation of cis-7-(benzyloxy)-1,2,3,3a,5,9b-hexahydroisochromeno[3,4-c]pyrrole

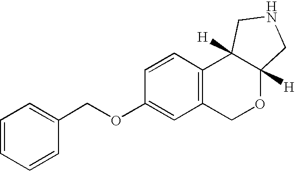

cis-2-Benzyl-7-(benzyloxy)-1,2,3,3a,5,9b-hexahydroisochromeno[3,4-c]pyrrole (3.63 mmol, 1.35 g) was dissolved in toluene (20 ml) and split between 4 large microwave vials. 1-Chloroethyl chloroformate (18.17 mmol, 1.961 mL, 2.60 g) was also split into 4 equal amounts and added to each vial. A small amount of acetonitrile (˜1 mL) was also added to each vial to aid microwave heating. Each vial was heated by microwave irradiation at 160° C. for 15 minutes. Methanol (5 mL) was then added to each vial and the mixture was subjected to microwave heating at 160° C. for 5 minutes. The mixture was then concentrated and loaded onto a pre-acidified SCX column using methanol. The product was eluted with 2M NH₃ in methanol and then concentrated to afford the crude cis-7-(benzyloxy)-1,2,3,3a,5,9b-hexahydroisochromeno[3,4-c]pyrrole (93% yield), EI-MS: m/z=282.5 [M+H]⁺.

### 25.12 Preparation of cis-tert-butyl 7-hydroxy-1,3,3a,9b-tetrahydroisochromeno[3,4-c]pyrrole-2(5H)-carboxylate

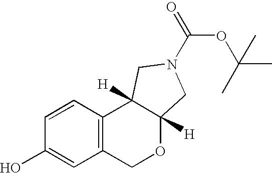

Triethylamine (6.75 mmol, 0.949 mL, 683 mg), Boc anhydride (6.75 mmol, 1474 mg) and 4-(N,N-dimethylamino)pyridine (0.844 mmol, 103 mg) were added to a solution of cis-7-(benzyloxy)-1,2,3,3a,5,9b-hexahydroisochromeno[3,4-c]pyrrole (3.38 mmol, 950 mg) in dichloromethane (16.900 mL), and stirred at room temperature for 4 hours. The reaction mixture was diluted with dichloromethane and washed with 10% aqueous citric acid. Collected organic phase was dried (MgSO₄), filtered and concentrated under reduced pressure to afford crude oil. Dry loaded onto Flourasil and purified using combiflash Retrieve (Si column, 120 g) eluting with toluene then 5 to 10% ethyl acetate in toluene to elute product. Fractions containing product were concentrated to afford cis-tert-butyl 7-(benzyloxy)-1,3,3a,9b-tetrahydroisochromeno[3,4-c]pyrrole-2(5H)-carboxylate (62% yield), EI-MS: m/z=382.0 [M+H]⁺. To cis-tert-butyl 7-(benzyloxy)-1,3,3a,9b-tetrahydroisochromeno[3,4-c]pyrrole-2(5H)-carboxylate (2.097 mmol, 0.8 g) and 10% palladium on carbon (0.752 mmol, 0.08 g) under inert atmosphere was added ethanol (20.97 ml). The reaction mixture was then stirred vigorously under an atmosphere of hydrogen (balloon) for 16 hours. Removed spent catalyst by filtering through celite, washing with methanol. Concentrated filtrate to afford cis-tert-butyl 7-hydroxy-1,3,3a,9b-tetrahydroisochromeno[3,4-c]pyrrole-2(5H)-carboxylate (69% yield), EI-MS: m/z=292.4 [M+H]⁺.

### 25.13 Preparation of cis-7-methoxy-1,2,3,3a,5,9b-hexahydroisochromeno[3,4-c]pyrrole

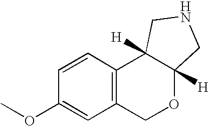

Iodomethane (0.178 mmol) was added to a mixture of cis-tert-butyl 7-hydroxy-1,3,3a,9b-tetrahydroisochromeno[3,4-c]pyrrole-2(5H)-carboxylate (26 mg, 0.089 mmol) and K₂CO₃ (62 mg) in acetone (6 ml) and the mixture heated at 60° C. with stirring for 24 h. Diethylamine (0.2 ml) was added and the reactions stirred at 60° C. for 3 h then the mixture was filtered and concentrated in vacuo. The residue was partitioned between 1M HCl (2 ml) and dichloromethane (3 ml). The dichloromethane phase was separated and concentrated in vacuo to give a residue. Flash chromatography using ethyl acetate in heptane (50%) as the eluent afforded a residue (18 mg). Dichloromethane (3 ml) and trifluoroacetic acid (0.5 ml) was added to the residue and the mixture was stirred for 2 h then concentrated in vacuo to give a residue. The residue was loaded onto a pre-acidified SCX column, washed with methanol and eluted with 2M NH₃ in methanol. The basic eluent was concentrated in vacuo and the residue purified by basic prep-LCMS to afford cis-7-methoxy-1,2,3,3a,5,9b-hexahydroisochromeno[3,4-c]pyrrole (26.0% yield), EI-MS: m/z=206.3 [M+H]⁺.

### EXAMPLE 26

### cis-6-Chloro-7-methoxy-1,2,3,3a,5,9b-hexahydroisochromeno[3,4-c]pyrrole hydrochloride

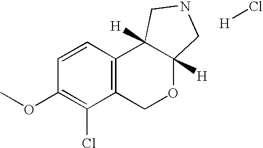

cis-tert-Butyl 7-hydroxy-1,3,3a,9b-tetrahydroisochromeno[3,4-c]pyrrole-2(5H)-carboxylate (0.583 mmol, 170 mg) was dissolved in acetonitrile (5.835 mL) and N-chlorosuccinimide (0.613 mmol, 82 mg) added. The reaction mixture was stirred in a sealed reaction vial at 82° C. for 3 hours. The mixture was cooled to room temperature and the solvent removed by nitrogen blow down. The mixture was partitioned between ethyl acetate and water, dried (MgSO₄) and concentrated to afford crude material. The products were purified by flash chromatography (Si, RediSep, 12 g), eluting with ethyl acetate in heptane (0 to 30%) to afford four fractions that were each concentrated to dryness. LCMS analysis revealed fraction 1 was dichlorinated product, fraction 2 was mono chlorinated, fraction 3 and fraction 4 were both mono-chlorinated. To fraction 3 was added acetonitrile (2 mL), iodomethane (35 uL) and K₂CO₃ (30 mg). The mixture was sealed in a reaction vial and heated at 60° C. for 3 hours. The solvent was then removed by nitrogen blow-down and the crude residue was partitioned between saturated aqueous NaHCO₃ (2 mL) and DCM (2 mL). The chlorinated phase was collected through a hydrophobic frit and TFA (0.5 mL) added to the filtrate. After stirring for 1 hour the TFA/DCM mixture was concentrated and the free base obtained by ion exchange chromatography (SCX, 0.5 g). Final purification by prep-LCMS and conversion to the HCl salt (HCl in ether) afforded cis-6-chloro-7-methoxy-1,2,3,3a,5,9b-hexahydroisochromeno[3,4-c]pyrrole hydrochloride (4.7 mg), EI-MS: m/z=240.3 and 242.6 [M+H]⁺.

### EXAMPLE 27

### cis-7-Ethyl-1,2,3,3a,5,9b-hexahydroisochromeno[3,4-c]pyrrole

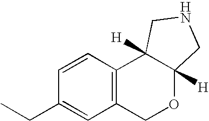

### 27.1 Preparation of cis-tert-butyl 7-(trifluoromethylsulfonyloxy)-1,3,3a,9b-tetrahydroisochromeno[3,4-c]pyrrole-2(5H)-carboxylate

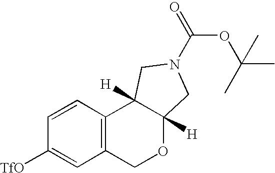

To a solution of cis-tert-butyl 7-hydroxy-1,3,3a,9b-tetrahydroisochromeno[3,4-c]pyrrole-2(5H)-carboxylate (0.343 mmol, 100 mg) in tetrahydrofuran (3.432 mL) was added sodium hydride (0.412 mmol, 9.89 mg) followed by N-phenyltrifluoromethanesulfonimide (0.343 mmol, 123 mg). The resultant suspension was stirred at ambient temperature for 16 hours and then tetrahydrofuran was removed and the residue partitioned between dichloromethane and NaHCO₃ (aq). The organic layer was collected via a hydrophobic frit and concentrated under reduced pressure to afford a residue (140 mg). The residue was loaded onto a flash column (12 g Si, RediSep) and eluted with a stepped solvent system of 10 to 30% ethyl acetate in heptane to afford cis-tert-butyl 7-(trifluoromethylsulfonyloxy)-1,3,3a,9b-tetrahydroisochromeno[3,4-c]pyrrole-2(5H)-carboxylate (70.2% yield), EI-MS: m/z=368.0 [M−^{t}Bu+H]⁺.

### 27.2 Preparation of cis-tert-butyl 7-vinyl-1,3,3a,9b-tetrahydroisochromeno[3,4-c]pyrrole-2(5H)-carboxylate

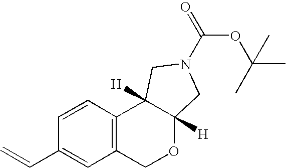

Tetrakis(triphenylphosphine)palladium (0) (2.95 μmol, 3.41 mg) was added in one portion to a mixture of cis-tert-butyl 7-(trifluoromethylsulfonyloxy)-1,3,3a,9b-tetrahydroisochromeno[3,4-c]pyrrole-2(5H)-carboxylate (0.118 mmol, 50 mg), 2,4,6-trivinylcyclotriboroxine pyridine complex (0.18 mmol, 28.4 mg) and potassium carbonate (0.142 mmol, 19.58 mg) in a degassed 1,2-dimethoxyethane (0.885 mL) and water (0.295 mL) mixture. The mixture was heated at 100° C. for 1.5 hours and then allowed to cool to room temperature, diluted with brine and extracted with ethyl acetate (×3). The combined organic extracts were dried (MgSO₄), filtered and concentrated under reduced pressure to afford the crude residue. Purification by flash chromatography (Si 2 g, RediSep) eluting with 20% ethyl acetate in heptane afforded cis-tert-butyl 7-vinyl-1,3,3a,9b-tetrahydroisochromeno[3,4-c]pyrrole-2(5H)-carboxylate (45% yield), EI-MS: m/z=246.4 [M−^{t}Bu+H]⁺.

### 27.3 Preparation of cis-7-ethyl-1,2,3,3a,5,9b-hexahydroisochromeno[3,4-c]pyrrole

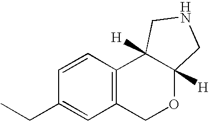

To cis-tert-butyl 7-vinyl-1,3,3a,9b-tetrahydroisochromeno[3,4-c]pyrrole-2(5H)-carboxylate (0.053 mmol, 16 mg) and 10% palladium on carbon (0.053 mmol, 2 mg) under an inert atmosphere was added ethanol (5.3 mL). The reaction mixture was then stirred vigorously under an atmosphere of hydrogen (balloon) for 16 hours. Removed spent catalyst by filtering through celite and washing with methanol. The filtrate was concentrated to afford crude product. The crude product was dissolved in dichloromethane (2 mL) and trifluoroacetic acid (0.2 mL) added. After stirring for 1 hour the sample was concentrated and purified by ion exchange chromatography (SCX, 0.5 g) to afford cis-7-ethyl-1,2,3,3a,5,9b-hexahydroisochromeno[3,4-c]pyrrole (57.4% yield), EI-MS: m/z=204.1 [M+H]⁺.

### EXAMPLE 28

### cis-7-Methyl-1,2,3,3a,5,9b-hexahydroisochromeno[3,4-c]pyrrole

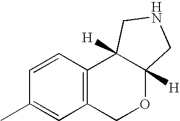

A mixture of cis-tert-butyl 7-(trifluoromethylsulfonyloxy)-1,3,3a,9b-tetrahydroisochromeno[3,4-c]pyrrole-2(5H)-carboxylate (0.118 mmol, 50 mg), trimethylboroxine (0.236 mmol, 33 μL, 29.6 mg), tetrakis(triphenylphosphine)palladium (0) (0.012 mmol, 13.65 mg) and potassium carbonate (0.236 mmol, 32.6 mg) in degassed dioxane (2 mL) were subjected to microwave irradiation at 120° C. for 20 minutes. The reaction mixture was partitioned between ethyl acetate and water. The organic extracts were combined, dried (MgSO₄), filtered and concentrated under reduced pressure to afford the crude residue. Purification by flash chromatography using 5 to 30% ethyl acetate in heptane as the eluent afforded the Boc-protected product (19 mg), EI-MS: m/z=290.5 [M+H]⁺. The Boc-protected product (19 mg) was dissolved in dichloromethane (2 mL) and trifluoroacetic acid (0.1 mL) was added. The resultant solution was stirred at ambient temperature for 2 hours and then the solvent removed by nitrogen blowdown. The product was purified by ion exchange chromatography (SCX, 500 mg) to afford pure cis-7-methyl-1,2,3,3a,5,9b-hexahydroisochromeno[3,4-c]pyrrole (52.1% yield), EI-MS: m/z=190.6 [M+H]⁺.

### EXAMPLE 29

### cis-2-Benzyl-8-methoxy-6-methyl-1,2,3,3a,5,9b-hexahydroisochromeno[3,4-c]pyrrole

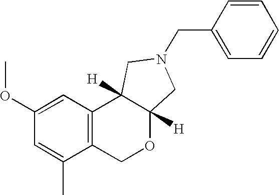

### 29.1 Preparation of 2-cyano-3-(4-methoxy-2-methylphenyl)acrylic acid

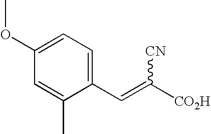

A stirred mixture of 4-methoxy-2-methylbenzaldehyde (133 mmol, 20 g), 2-cyanoacetic acid (133 mmol, 11.33 g), ammonium acetate (26.6 mmol, 2.053 g), pyridine (233 mmol, 18.85 ml, 18.43 g) and toluene (100 ml) was refluxed using a Dean-Stark apparatus until one molar equivalent of water was collected. The reaction mixture was allowed to cool and then concentrated to form a residue that was partitioned between ethyl acetate and 5N hydrochloric acid. The combined organics were washed with water, dried (Na₂SO₄) and concentrated in vacuo to afford 2-cyano-3-(4-methoxy-2-methylphenyl)acrylic acid (99% yield, 28.77 g), ¹H NMR (400 MHz, DMSO) ppm 8.40 (1H, s, CHCCNCO₂H), 8.13 (1H, d, ArH), 6.95-7.01 (2H, m, 2×ArH), 3.84 (3H, s, OCH₃), 3.1-3.5 (1H, br. s, OH) and 2.42 (3H, s, CH₃).

### 29.2 Preparation of 2-cyano-3-(4-methoxy-2-methyl phenyl)propanoic acid

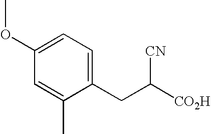

Sodium borohydride (331 mmol, 12.53 g) was added, in portions over a period of 45 minutes, to a stirred suspension of 2-cyano-3-(4-methoxy-2-methylphenyl)acrylic acid (132 mmol, 28.77 g), sodium bicarbonate (146 mmol, 12.24 g) and water (265 ml) at 0° C. The reaction mixture was allowed to warm to room temperature and stirred for 2 hours before acidifying with hydrochloric acid and extracting the product with ether. The combined organics were dried over sodium sulphate, filtered and evaporated under reduced pressure to afford 2-cyano-3-(4-methoxy-2-methylphenyl)propanoic acid (97% yield, 28.16 g), ¹H NMR (400 MHz, CDCl₃) ppm 8.99 (1H, br. s, OH), 7.16 (1H, d, ArH), 6.70-6.78 (2H, m, 2×ArH), 3.79 (3H, s, OCH₃), 3.70 (1H, dd, CHCNCO₂H), 3.32 (1H, dd, CH₂CH), 3.16 (1H, dd, CH₂CH) and 2.34 (3H, s, CH₃).

### 29.3 Preparation of 3-(4-methoxy-2-methyl phenyl)propanenitrile

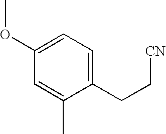

A solution of 2-cyano-3-(4-methoxy-2-methylphenyl)propanoic acid (128 mmol, 28.16 g) in dimethylacetamide (56 mL) was heated at 150° C. for 2.5 h. The reaction mixture was poured into water and the product was extracted with diethyl ether. The combined organics were washed with water, dried (Na₂SO₄) and concentrated under reduced pressure to afford 3-(4-methoxy-2-methylphenyl)propanenitrile (100% yield, 22 g), ¹H NMR (400 MHz, CDCl₃) ppm 7.08 (1H, d, ArH), 6.70-6.75 (2H, m, 2×ArH), 3.77 (3H, s, OCH₃), 2.91 (2H, t, CH₂CN), 2.53 (2H, t, CH₂CH₂) and 2.30 (3H, s, CH₃).

### 29.4 Preparation of 3-(5-bromo-4-methoxy-2-methyl phenyl)propanenitrile

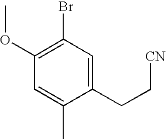

A solution of bromine (148 mmol, 7.59 ml, 23.68 g) in chloroform (50.0 ml) was added dropwise over a period of 1 h to a stirred mixture of 3-(4-methoxy-2-methylphenyl)propanenitrile (123 mmol, 21.64 g), sodium acetate (123 mmol, 10.13 g) and chloroform (100 ml), at room temperature. The mixture was stirred for 3 h then washed with water. The organic phase was separated, dried (Na₂SO₄) and concentrated in vacuo to afford a crude gum. Recrystallisation from ethanol afforded 3-(5-bromo-4-methoxy-2-methylphenyl)propanenitrile (15.2 g), ¹H-NMR (400 MHz, CDCl₃) ppm 7.31 (1H, s, ArH), 6.71 (1H, s, ArH), 3.87 (3H, s, OCH₃), 2.89 (2H, t, CH₂CN), 2.55 (2H, t, CH₂CH₂) and 2.31 (3H, s, CH₃).

### 29.5 Preparation of 5-methoxy-3-methyl-1,2-dihydrocyclobutabenzene-1-carbonitrile

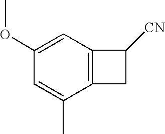

Ammonia gas was condensed into the flask from a cylinder until approximately the required volume was present (˜125 mL). Commercial NaNH₂ (243 mmol, 9.47 g) was added to the ammonia at −78° C. and after stirring for 10 minutes 3-(5-bromo-4-methoxy-2-methylphenyl)propanenitrile (61.8 mmol, 15.70 g) was added over a 5 minute period. The mixture was allowed to warm such that the resultant mixture was stirred at reflux for 3 h before being neutralised with solid ammonium nitrate (268 mmol, 21.41 g) and allowed to stand overnight under a flow of nitrogen. All the ammonia was evaporated and water was added to the solid residue and the products extracted with dichloromethane (×3). The combined organics were washed with dilute hydrochloric acid (5%), followed by water. The organics were dried with sodium sulfate and concentrated in vacuo to afford crude product as a brown residue (11.29 g). Flash chromatography of the residue using 5% ethyl acetate in heptane as the eluent afforded 5-methoxy-3-methyl-1,2-dihydrocyclobutabenzene-1-carbonitrile (8.24 g), ¹H NMR (400 MHz, CDCl₃) ppm 6.69 (1H, s, ArH), 6.62 (1H, s, ArH), 4.13 (1H, dd, CHCN), 3.77 (3H, s, OCH₃), 3.53 (1H, dd, CH₂CH), 3.40 (H, dd, CH₂CH) and 2.17 (3H, s, CH).

### 29.6 Preparation of 5-methoxy-3-methyl-1,2-dihydrocyclobutabenzene-1-carboxylic acid

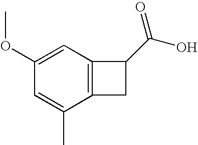

A solution of 5-methoxy-3-methyl-1,2-dihydrocyclobutabenzene-1-carbonitrile (29.2 mmol, 5.05 g) and KOH (146 mmol, 8.18 g) in ethanol (97 ml) and water (19.44 ml) was refluxed for 2.5 h. After evaporation of the organic solvent under reduced pressure, the aqueous residue was diluted with 2N aqueous NaOH (1 L) and washed with Et₂O (2×750 mL). The aqueous phase was acidified with 5N aqueous HCl, during which a precipitate formed that was collected by filtration and dried in vacuo to afford 5-methoxy-3-methyl-1,2-dihydrocyclobutabenzene-1-carboxylic acid (93% yield, 5.22 g), ¹H NMR (400 MHz, CDCl₃) ppm 6.60 (1H, s, ArH), 6.59 (1H, s, ArH), 4.15 (1H, dd, CHCO₂H), 3.73 (3H, s, OCH₃), 3.21-3.32 (2H, m, CH₂CH) and 2.14 (3H, s, CH₃).

### 29.7 Preparation of cis-2-benzyl-8-methoxy-6-methyl-1,2,3,3a,5,9b-hexahydroisochromeno[3,4-c]pyrrole

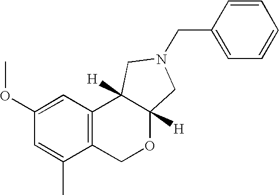

Similar protocols to procedures in Example 1.6 to 1.9 were employed, using 5-methoxy-3-methyl-1,2-dihydrocyclobutabenzene-1-carboxylic acid to afford cis-2-benzyl-8-methoxy-6-methyl-1,2,3,3a,5,9b-hexahydroisochromeno[3,4-c]pyrrole, EI-MS: m/z=310.1 [M+H]⁺.

### EXAMPLE 30

### cis-8-Methoxy-6-methyl-1,2,3,3a,5,9b-hexahydroisochromeno[3,4-c]pyrrole hydrochloride

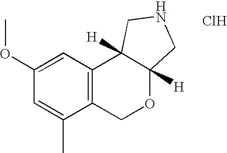

A solution of cis-2-benzyl-8-methoxy-6-methyl-1,2,3,3a,5,9b-hexahydroisochromeno[3,4-c]pyrrole (0.129 mmol, 40 mg) and 1-chloroethyl chloroformate (0.646 mmol, 0.070 mL, 92 mg) in toluene (2 ml) was subjected to microwave irradiation at 160° C. for 15 minutes, then methanol was added to the mixture which was subjected to further microwave irradiation at 160° C. for 5.5 minutes. The mixture was concentrated, loaded onto a pre-acidified SCX column using methanol and the product eluted with 2M NH₃ in methanol. The eluent was concentrated to afford a residue which was purified by flash chromatography using 2 M NH₃ in methanol and dichoromethane (1% to 5%) as the eluent. The product fractions were combined and concentrated in vacuo then dissolved in MeOH and 2 M HCl in ether. The mixture was concentrated under reduced pressure to afford cis-8-methoxy-6-methyl-1,2,3,3a,5,9b-hexahydroisochromeno[3,4-c]pyrrole hydrochloride (79% yield, 26 mg), EI-MS: m/z=220.4 [M+H]⁺.

### EXAMPLE 31

### trans-2-Benzyl-8-methoxy-6-methyl-1,2,3,3a,5,9b-hexahydroisochromeno[3,4-c]pyrrole

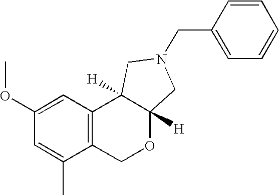

Similar reaction protocols for procedures in Example 29.7 were repeated for trans-2-benzyl-8-methoxy-6-methyl-3,3a,5,9b-tetrahydroisochromeno[3,4-c]pyrrol-1(2H)-one (0.618 mmol, 200 mg) to afford trans-2-benzyl-8-methoxy-6-methyl-1,2,3,3a,5,9b-hexahydroisochromeno[3,4-c]pyrrole, EI-MS: m/z=310.1 [M+H]⁺.

### EXAMPLE 32

### trans-8-Methoxy-6-methyl-1,2,3,3a,5,9b-hexahydroisochromeno[3,4-c]pyrrole hydrochloride

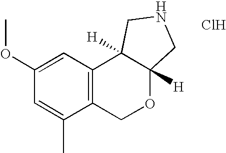

Similar protocols to that used for Example 30 were employed for trans-2-benzyl-8-methoxy-6-methyl-1,2,3,3a,5,9b-hexahydroisochromeno[3,4-c]pyrrole (190 mg) to afford trans-8-methoxy-6-methyl-1,2,3,3a,5,9b-hexahydroisochromeno[3,4-c]pyrrole hydrochloride (14% yield), EI-MS: m/z=220.4 [M+H]⁺.

### EXAMPLE 33

### cis-6,8-Dimethyl-1,2,3,3a,5,9b-hexahydroisochromeno[3,4-c]pyrrole hydrochloride

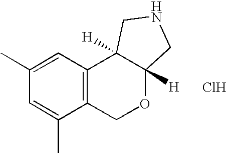

### 33.1 Preparation of cis-ethyl 8-hydroxy-6-methyl-1,3,3a,9b-tetrahydroisochromeno[3,4-c]pyrrole-2(5H)-carboxylate

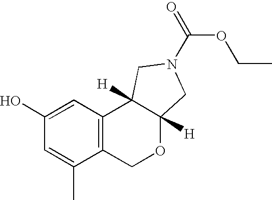

Sodium bicarbonate (25.08 mmol, 2.107 g) and ethyl chloroformate (6.02 mmol, 0.653 g) were added to a solution of cis-8-methoxy-6-methyl-1,2,3,3a,5,9b-hexahydroisochromeno[3,4-c]pyrrole (5.02 mmol, 1.1 g) in a THF (12.54 ml) and water (12.54 ml) mixture, and the mixture was stirred at room temperature for 16 hours. The reaction mixture was then quenched by the addition of an aqueous HCl solution (17 mL, 1 M) and the product was extracted with EtOAc (3×20 mL). The combined extracts were dried over MgSO₄ and concentrated in vacuo to give a crude oil that was purified by silica column chromatography (40 g silica, eluting with 10% EtOAc in heptane) to afford cis-ethyl 8-methoxy-6-methyl-1,3,3a,9b-tetrahydroisochromeno[3,4-c]pyrrole-2(5H)-carboxylate (1.23 g). cis-Ethyl 8-methoxy-6-methyl-1,3,3a,9b-tetrahydroisochromeno[3,4-c]pyrrole-2(5H)-carboxylate (200 mg, 0.69 mmol) was then dissolved in DCM (5 mL) and BBr₃ (1 M in DCM, 1.72 mmol, 1.72 mL) was added. The resultant solution was stirred overnight at room temperature. Excess BBr₃ was then quenched by dropwise addition of water (10 mL). The phases were separated and the organic extract was washed with saturated NaHCO₃ (50 mL) and brine (50 mL). The organic extract was dried over MgSO₄ and concentrated in vacuo to afford a crude product that was purified by silica column chromatography (12 g silica, eluting with EtOAc in heptane) to afford cis-ethyl 8-hydroxy-6-methyl-1,3,3a,9b-tetrahydroisochromeno[3,4-c]pyrrole-2(5H)-carboxylate (66% yield).

### 33.2 Preparation of cis-ethyl 6-methyl-8-(trifluoromethylsulfonyloxy)-1,3,3a,9b-tetrahydroisochromeno[3,4-c]pyrrole-2(5H)-carboxylate

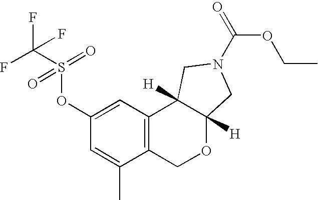

To a solution of cis-ethyl 8-hydroxy-6-methyl-1,3,3a,9b-tetrahydroisochromeno[3,4-c]pyrrole-2(5H)-carboxylate (1.551 mmol, 430 mg) in tetrahydrofuran (15.5 mL) was added sodium hydride (1.861 mmol, 74.4 mg) followed by N-phenyltrifluoromethanesulfonimide (1.628 mmol, 582 mg). The resultant suspension was stirred at ambient temperature for 16 hours and then THF was removed and the residue partitioned between DCM and saturated NaHCO₃ (aq). The organic layer was collected via a hydrophobic frit and concentrated under reduced pressure to afford a residue. The residue was flash chromatographed using ethyl acetate in heptane (10 to 40%) as the eluent to afford cis-ethyl 6-methyl-8-(trifluoromethylsulfonyloxy)-1,3,3a,9b-tetrahydroisochromeno[3,4-c]pyrrole-2(5H)-carboxylate (430 mg).

### 33.3 Preparation of cis-6,8-dimethyl-1,2,3,3a,5,9b-hexahydroisochromeno[3,4-c]pyrrole hydrochloride

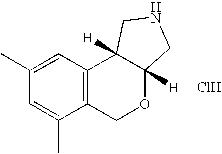

A mixture of cis-ethyl 6-methyl-8-(trifluoromethylsulfonyloxy)-1,3,3a,9b-tetrahydroisochromeno[3,4-c]pyrrole-2(5H)-carboxylate (0.098 mmol, 40 mg), trimethylboroxine (0.195 mmol, 0.027 mL, 24.53 mg), tetrakis(triphenylphosphine)palladium(0) (9.77 μmol, 11.29 mg) and potassium carbonate (0.195 mmol, 27.0 mg) in degassed dioxane (2 mL) were subjected to microwave irradiation at 120° C. for 20 minutes. After analysis by LCMS the reaction mixture was partitioned between ethyl acetate and water. The organic layer was dried (MgSO₄), filtered and concentrated under reduced pressure to afford a crude residue. Purification by flash chromatography (Si, 2 g, RediSep) eluting with ethyl acetate in heptane (5 to 30%) afforded cis-ethyl 6,8-dimethyl-1,3,3a,9b-tetrahydroisochromeno[3,4-c]pyrrole-2(5H)-carboxylate (22 mg). cis-Ethyl 6,8-dimethyl-1,3,3a,9b-tetrahydroisochromeno[3,4-c]pyrrole-2(5H)-carboxylate (22 mg) was dissolved in methanol (0.5 mL) and transferred to a 2 mL microwave vial. A solution of KOH (80 mg) in water (1.0 mL) was added and the resultant mixture heated by microwave irradiation at 150° C. for 30 minutes. The mixture was diluted with water (2 mL) and the product extracted with DCM (3×3 mL). The combined organics were collected through a hydrophobic frit and concentrated to dryness under reduced pressure. The crude product was purified by ion exchange chromatography (SCX, 0.5 g). The free base was converted to the hydrochloride salt by dissolving in DCM and shaking with HCl in ether before concentrating to afford pure cis-6,8-dimethyl-1,2,3,3a,5,9b-hexahydroisochromeno[3,4-c]pyrrole hydrochloride (12.4 mg, 53% yield), EI-MS: m/z=204.1 [M+H]⁺.

### EXAMPLE 34

### cis-8-Ethyl-6-methyl-1,2,3,3a,5,9b-hexahydroisochromeno[3,4-c]pyrrole hydrochloride

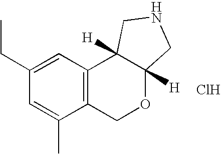

A similar procedure to that in Example 14 was employed, using 2,4,6-trivinylcyclotriboroxane pyridine complex. The ethyl carbamate protecting group was removed in a similar way to that in Example 33.2 to afford cis-8-ethyl-6-methyl-1,2,3,3a,5,9b-hexahydroisochromeno[3,4-c]pyrrole hydrochloride (57% yield), EI-MS: m/z=218.4 [M+H]⁺.

### EXAMPLE 35

### cis-2-Benzyl-7-bromo-6-chloro-1,2,3,3a,5,9b-hexahydroisochromeno[3,4-c]pyrrole

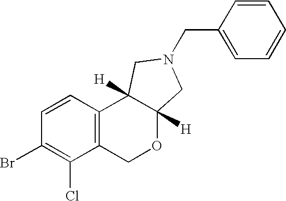

### 35.1 Preparation of 4-bromo-3-chloro-1,2-dihydrocyclobutabenzene-1-carboxylic acid & 6-bromo-3-chloro-1,2-dihydrocyclobutabenzene-1-carboxylic acid

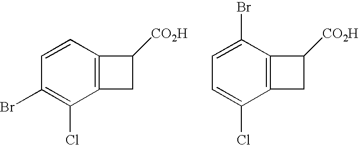

N-Bromosuccinimide (27.4 mmol, 4.87 g) was added to a solution of 3-chloro-1,2-dihydrocyclobutabenzene-1-carboxylic acid (27.4 mmol, 5.0 g) in sulfuric acid (25 ml) and the mixture was stirred at ambient temperature for 16 h. The reaction mixture was poured into ice-water and extracted with ether. The organics were extracted with 2N NaOH (×2) and the combined aqueous layer was washed with ether, then acidified with 5N HCl and extracted with ether (×2). This organic layer was washed with brine, dried (Na₂SO₄) and concentrated in vacuo to afford a crystalline residue (6.81 g). The solid residue was recrystallised with acetonitrile to afford 4-bromo-3-chloro-1,2-dihydrocyclobutabenzene-1-carboxylic acid (4 g) and the resultant filtrate was purified by flash chromatography using ethyl acetate in heptane (30% to 60%) to afford crude 6-bromo-3-chloro-1,2-dihydrocyclobutabenzene-1-carboxylic acid (72 mg).

### 35.2 Preparation of cis-2-benzyl-7-bromo-6-chloro-1,2,3,3a,5,9b-hexahydroisochromeno[3,4-c]pyrrole

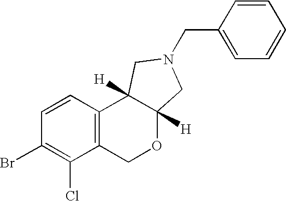

Similar protocols to procedures 1.6 to 1.9 were employed, using 4-bromo-3-chloro-1,2-dihydrocyclobutabenzene-1-carboxylic acid to afford cis-2-benzyl-7-bromo-6-chloro-1,2,3,3a,5,9b-hexahydroisochromeno[3,4-c]pyrrole, EI-MS: m/z=380.1 [M+H]⁺.

### EXAMPLE 36

### trans-2-Benzyl-7-bromo-6-chloro-1,2,3,3a,5,9b-hexahydroisochromeno[3,4-c]pyrrole

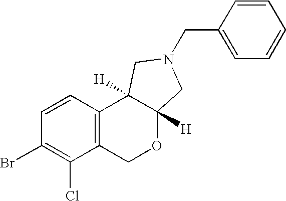

Similar protocols to procedures 1.6 to 1.9 were employed, using 4-bromo-3-chloro-1,2-dihydrocyclobutabenzene-1-carboxylic acid to afford trans-2-benzyl-7-bromo-6-chloro-1,2,3,3a,5,9b-hexahydroisochromeno[3,4-c]pyrrole, EI-MS: m/z=380.5 [M+H]⁺.

### EXAMPLE 37

### cis-7-Bromo-6-chloro-1,2,3,3a,5,9b-hexahydroisochromeno[3,4-c]pyrrole hydrochloride

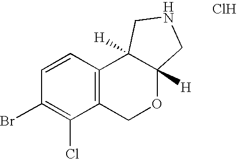

Similar protocols to procedures in Example 30 were employed, using cis-2-benzyl-7-bromo-6-chloro-1,2,3,3a,5,9b-hexahydroisochromeno[3,4-c]pyrrole (0.132 mmol, 50 mg) to afford cis-7-bromo-6-chloro-1,2,3,3a,5,9b-hexahydroisochromeno[3,4-c]pyrrole hydrochloride (77%), EI-MS: m/z=288.0 & 290.0 [M+H]⁺.

### EXAMPLE 38

### trans-7-Bromo-6-chloro-1,2,3,3a,5,9b-hexahydroisochromeno[3,4-c]pyrrole hydrochloride

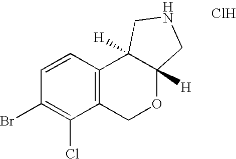

Similar protocols to procedures in Example 30 were employed, using trans-2-benzyl-7-bromo-6-chloro-1,2,3,3a,5,9b-hexahydroisochromeno[3,4-c]pyrrole (0.032 mmol, 12 mg) to afford trans-7-bromo-6-chloro-1,2,3,3a,5,9b-hexahydroisochromeno[3,4-c]pyrrole hydrochloride (80%), EI-MS: m/z=290.0 [M+H]⁺.

### EXAMPLE 39

### cis-6,7-Dichloro-1,2,3,3a,5,9b-hexahydroisochromeno[3,4-c]pyrrole hydrochloride

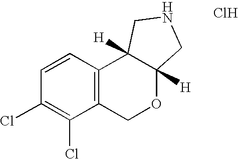

### 39.1 Preparartion of cis-2-benzyl-6,7-dichloro-3,3a,5,9b-tetrahydroisochromeno[3,4-c]pyrrol-1(2H)-one

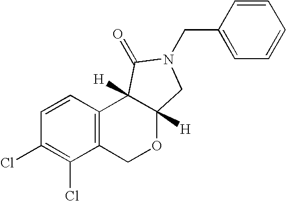

A mixture of cis-2-benzyl-7-bromo-6-chloro-3,3a,5,9b-tetrahydroisochromeno[3,4-c]pyrrol-1(2H)-one (0.183 mmol, 72 mg), and nickel (II) chloride hexahydrate (0.733 mmol, 174 mg) in N-methyl-2-pyrrolidinone (2 ml) was subjected to microwave irradiation at 180° C. for 10 minutes, then at 210° C. for 60 minutes. Water was added and the mixture extracted with ethyl acetate, washed with brine, dried (Na₂SO₄) and concentrated in vacuo. Flash chromatography of the residue using ethyl acetate in heptane (30% to 50%) as the eluent afforded cis-2-benzyl-6,7-dichloro-3,3a,5,9b-tetrahydroisochromeno[3,4-c]pyrrol-1(2H)-one (39.4 mg, 62% yield), EI-MS: m/z=348.4 & 350.6 [M+H]⁺.

### 39.2 Preparartion of cis-2-benzyl-6,7-dichloro-1,2,3,3a,5,9b-hexahydroisochromeno[3,4-c]pyrrole

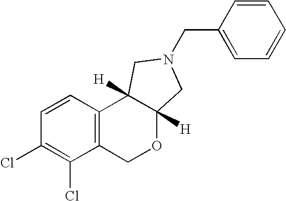

Similar protocols to procedures in Example 1.9 were employed, using cis-2-benzyl-6,7-dichloro-3,3a,5,9b-tetrahydroisochromeno[3,4-c]pyrrol-1(2H)-one (0.109 mmol, 38 mg) to afford cis-2-benzyl-6,7-dichloro-1,2,3,3a,5,9b-hexahydroisochromeno[3,4-c]pyrrole (30.6 mg, 84%), EI-MS: m/z=334.1 & 336.1 [M+H]⁺.

### 39.3 Preparartion of cis-6,7-dichloro-1,2,3,3a,5,9b-hexahydroisochromeno[3,4-c]pyrrole hydrochloride

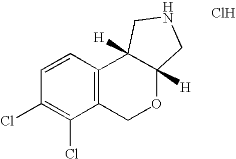

Similar protocols to procedures in Example 2 were employed, using cis-2-benzyl-6,7-dichloro-1,2,3,3a,5,9b-hexahydroisochromeno[3,4-c]pyrrole (0.06 mmol, 20 mg) to afford cis-6,7-dichloro-1,2,3,3a,5,9b-hexahydroisochromeno[3,4-c]pyrrole hydrochloride (77%), EI-MS: m/z=244.3 & 246.4 [M+H]⁺.

### EXAMPLE 40

### cis-2-Benzyl-6-chloro-3a-methyl-1,2,3,3a,5,9b-hexahydroisochromeno[3,4-c]pyrrole & trans-2-benzyl-6-chloro-3a-methyl-1,2,3,3a,5,9b-hexahydroisochromeno[3,4-c]pyrrole

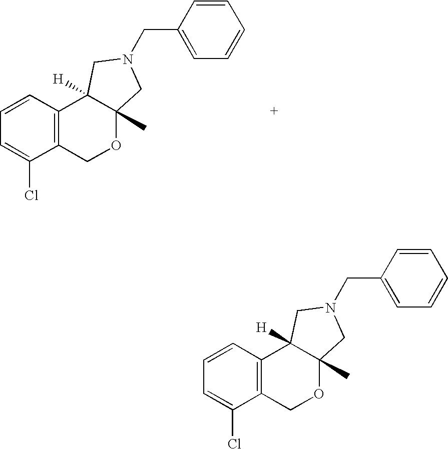

### 40.1 Preparation of 3-chloro-1,2-dihydrocyclobutabenzene-1-carboxylic acid

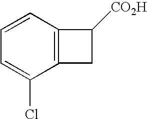

The 3-chloro-1,2-dihydrocyclobutabenzene-1-carboxylic acid was prepared using similar procedures as those in Example 1.1 to 1.5.

### 40.2 Preparation of N-benzyl-3-chloro-N-(2-hydroxypropyl)-1,2-dihydrocyclobutabenzene-1-carboxamide

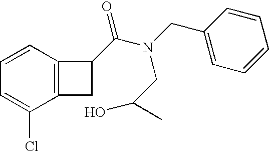

A mixture of 1-(benzylamino)propan-2-ol (12.32 mmol, 2.036 g), triethylamine (16.43 mmol, 2.290 ml, 1.662 g), 3-chloro-1,2-dihydrocyclobutabenzene-1-carboxylic acid (1.56 g, 8.21 mmol) and cyclophos in ethyl acetate (9.86 mmol, 5.87 ml, 6.27 g) in DCM was stirred at room temperature for 2 h. The reaction mixture was partitioned between dichloromethane and 2N aqueous HCl. The aqueous layer was extracted with dichloromethane and combined organic layers were washed with water then brine, dried (Na₂SO₄) and concentrated in vacuo. The residue was purified with silicagel column chromatography eluting with 30 to 50% ethyl acetate in heptane to afford N-benzyl-3-chloro-N-(2-hydroxypropyl)-1,2-dihydrocyclobutabenzene-1-carboxamide (96% yield).

### 40.3 Preparation of N-benzyl-3-chloro-N-(2-oxopropyl)-1,2-dihydrocyclobutabenzene-1-carboxamide

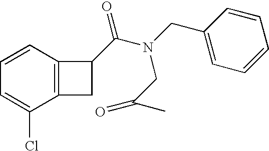

To a solution of N-benzyl-3-chloro-N-(2-hydroxypropyl)-1,2-dihydrocyclobutabenzene-1-carboxamide (2.56 g, 7.76 mmol) in dichloromethane (64.7 ml) was added a solution of Dess-Martin periodinane (62.1 mmol, 19.34 ml, 26.3 g) 15 wt % in dichloromethane. The mixture was stirred at room temperature for 3 h then a further 2.3 ml of Dess-Martin periodinane was added. The mixture was stirred for a further 1 h and then saturated aqueous NaHCO₃ was added and the mixture stirred for a further 30 min. The mixture was then extracted with dichloromethane (×3), washed with brine, dried (MgSO₄) and concentrated under reduced pressure to afford a residue. Flash chromatography using ethyl acetate in heptane (10% to 30%) as the eluent afforded N-benzyl-3-chloro-N-(2-oxopropyl)-1,2-dihydrocyclobutabenzene-1-carboxamide (76% yield), EI-MS: m/z=328.3 [M+H]⁺.

### 40.4 Preparation of trans-2-benzyl-6-chloro-3a-methyl-3,3a,5,9b-tetrahydroisochromeno[3,4-c]pyrrol-1(2H)-one & cis-2-benzyl-6-chloro-3a-methyl-3,3a,5,9b-tetrahydroisochromeno[3,4-c]pyrrol-1(2H)-one

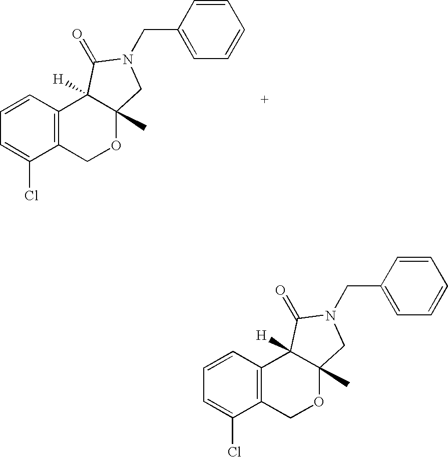

A solution of N-benzyl-3-chloro-N-(2-oxopropyl)-1,2-dihydrocyclobutabenzene-1-carboxamide (2.86 mmol, 936 mg) in bromobenzene (30 ml) in two large microwave vials, was irradiation at 210° C. for 30 minutes. The reaction mixture was loaded directly onto a Biotage® column and elueted with heptane followed by ethyl acetate-heptane 10% to 50% to afford trans-2-benzyl-6-chloro-3a-methyl-3,3a,5,9b-tetrahydroisochromeno[3,4-c]pyrrol-1(2H)-one (42.0% yield), followed by cis-2-benzyl-6-chloro-3a-methyl-3,3a,5,9b-tetrahydroisochromeno[3,4-c]pyrrol-1(2H)-one (37.4% yield)

### 40.5 Preparation of trans-2-benzyl-6-chloro-3a-methyl-1,2,3,3a,5,9b-hexahydroisochromeno[3,4-c]pyrrole

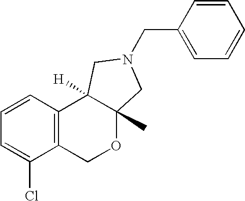

trans-2-Benzyl-6-chloro-3a-methyl-3,3a,5,9b-tetrahydroisochromeno[3,4-c]pyrrol-1(2H)-one (393 mg, 1.19 mmol) was dissolved in tetrahydrofuran (12 ml) and to the resultant solution was added borane-dimethylsulfide complex (8.39 mmol, 807 μl, 638 mg). The mixture was refluxed under nitrogen for 6 h then cooled to 5° C. and 5N aqueous HCl (3 ml) added. The mixture was refluxed for an addition 1.5 h and then left to stand overnight (16 h). After addition of excess saturated aqueous NaHCO₃, the mixture was extracted with EtOAc, the organics combined, washed with brine, dried (Na₂SO₄) and concentrated under reduced pressure to give a residue. The crude residue was purified with silicagel column chromatography eluting with 10 to 30% ethyl acetate in heptane to afford trans-2-benzyl-6-chloro-3a-methyl-1,2,3,3a,5,9b-hexahydroisochromeno[3,4-c]pyrrole (68% yield), EI-MS: m/z=314.1 [M+H]⁺.

### 40.6 Preparation of cis-2-benzyl-6-chloro-3a-methyl-1,2,3,3a,5,9b-hexahydroisochromeno[3,4-c]pyrrole

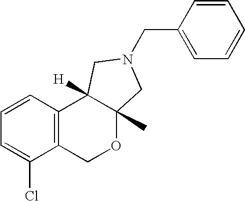

Similar protocols to procedures in Example 40.5 were employed, using cis-2-benzyl-6-chloro-3a-methyl-3,3a,5,9b-tetrahydroisochromeno[3,4-c]pyrrol-1(2H)-one (667 mg, 2.03 mmol) to afford cis-2-benzyl-6-chloro-3a-methyl-1,2,3,3a,5,9b-hexahydroisochromeno[3,4-c]pyrrole (93% yield), EI-MS: m/z=314.3 [M+H]⁺.

### EXAMPLE 41

### trans-6-Chloro-3a-methyl-1,2,3,3a,5,9b-hexahydroisochromeno[3,4-c]pyrrole hydrochloride

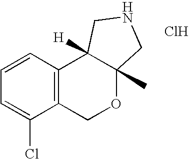

A solution of trans-2-benzyl-6-chloro-3a-methyl-1,2,3,3a,5,9b-hexahydroisochromeno[3,4-c]pyrrole (226 mg, 0.720 mmol) and 1-chloroethyl chloroformate (3.60 mmol, 389 μl, 515 mg) in toluene (3 ml) was subjected to microwave irradiation at 160° C. for 15 minutes, then methanol (1 ml) was added to the mixture and the mixture was subjected to microwave irradiation at 160° C. for 5.5 minutes. The combined mixture was then concentrated and loaded onto a pre-acidified SCX column using methanol. The product was eluted with 2M ammonia in methanol and then concentrated to afford the desired product. Flash chromatography using 10% methanol in dichloromethane, followed by 10% 2M NH₃ in MeOH in DCM afforded ˜150 mg of product, which was purified by basic prep-HPLC and then concentrated and converted to the HCl salt using HCl in ether to give trans-6-chloro-3a-methyl-1,2,3,3a,5,9b-hexahydroisochromeno[3,4-c]pyrrole hydrochloride (27% yield), EI-MS: m/z=224.3 [M+H]⁺.

### EXAMPLE 42

### cis-6-Chloro-3a-methyl-1,2,3,3a,5,9b-hexahydroisochromeno[3,4-c]pyrrole hydrochloride

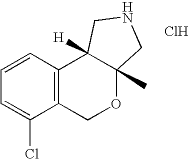

Similar protocols to procedures in Example 41 were employed, using cis-2-benzyl-6-chloro-3a-methyl-1,2,3,3a,5,9b-hexahydroisochromeno[3,4-c]pyrrole (556 mg, 1.7 mmol) to afford cis-6-chloro-3a-methyl-1,2,3,3a,5,9b-hexahydroisochromeno[3,4-c]pyrrole hydrochloride (78% yield), EI-MS: m/z=224.4 [M+H]⁺.

### EXAMPLE 43

### trans-7-Bromo-6-chloro-3a-methyl-1,2,3,3a,5,9b-hexahydroisochromeno[3,4-c]pyrrole hydrochloride

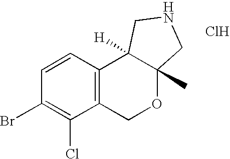

### 43.1 Preparation of N-benzyl-4-bromo-3-chloro-N-(2-hydroxypropyl)-1,2-dihydrocyclobutabenzene-1-carboxamide

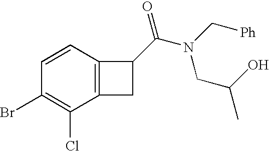

To a solution of 4-bromo-3-chloro-1,2-dihydrocyclobutabenzene-1-carboxylic acid (7.11 mmol, 1.86 g) and Cyclophos (50 wgt % in AcOEt) (9.25 mmol, 5.88 g) in DCM (30 ml) was added 1-(benzylamino)propan-2-ol (10.67 mmol, 1.763 g). Triethylamine (14.23 mmol, 1.997 ml, 1.440 g) was added and the reaction mixture was stirred at RT for 2 hr. The reaction mixture was quenched with 2 N HCl (30 mL) and the aqueous layer was extracted with DCM and combined organic extracts were washed with water, then brine, dried over Na₂SO₄ and concentrated in vacuo to afford a crude oil that was purified by silica column chromatography (40 g silica, eluting with heptane/EtOAc 4/1, 3/1, 2/1, then EtOAc) to afford N-benzyl-4-bromo-3-chloro-N-(2-hydroxypropyl)-1,2-dihydrocyclobutabenzene-1-carboxamide (1.91 g, 66%) as a white solid.

### 43.2 Preparation of N-benzyl-4-bromo-3-chloro-N-(2-oxopropyl)-1,2-dihydrocyclobutabenzene-1-carboxamide

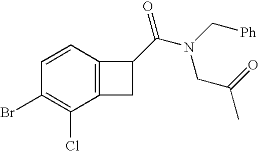

To a solution of N-benzyl-4-bromo-3-chloro-N-(2-hydroxypropyl)-1,2-dihydrocyclobutabenzene-1-carboxamide (3.38 mmol, 1.38 g) in DCM (15 ml) was added Dess-Martin Periodinane (15% in DCM) (5.07 mmol, 10.53 ml, 14.34 g) dropwise and the mixture was stirred at 20° C. for 1.5 h. Further Dess-Martin soln (1 mL) was added and stirring was continued for a further 45 min. Sat. aq sodium bicarbonate soln. (30 ml) was added and stirring continued for 1 h. The mixture was then filtered through Dicalite and the resulting filtrate was separated and the aqueous extract washed with DCM (3×15 ml). The combined organic extracts were concentrated in vacuo to give a crude oil that was purified by silica column chromatography (40 g silica, eluting with heptane, heptane/EtOAc 9/1-1/1 to afford N-benzyl-4-bromo-3-chloro-N-(2-oxopropyl)-1,2-dihydrocyclobutabenzene-1-carboxamide (1.2 g, 58%).

### 43.3 Preparation of cis-2-benzyl-7-bromo-6-chloro-3a-methyl-3,3a,5,9b-tetrahydroisochromeno[3,4-c]pyrrol-1(2H)-one and trans-2-benzyl-7-bromo-6-chloro-3a-methyl-3,3a,5,9b-tetrahydroisochromeno[3,4-c]pyrrol-1(2H)-one

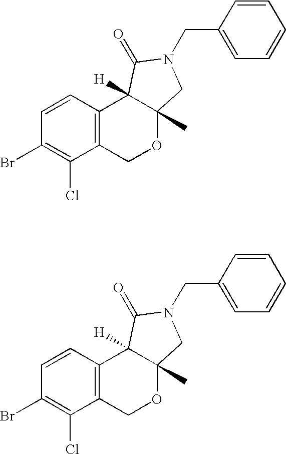

A solution of N-benzyl-4-bromo-3-chloro-N-(2-oxopropyl)-1,2-dihydrocyclobutabenzene-1-carboxamide (2.95 mmol, 1.2 g) in bromobenzene (30 mL) subjected to microwave irradiation at 210° C. for 30 minutes. The solvent was removed under vacuum to obtain a crude oil that was purified by silica column chromatography (40 g silica, eluting with 20%-50% (v/v) ethyl acetate in heptane) to give trans-2-benzyl-7-bromo-6-chloro-3a-methyl-3,3a,5,9b-tetrahydroisochromeno[3,4-c]pyrrol-1(2H)-one (325 mg, 27%), EI-MS: m/z=408.0 [M+H]⁺, then cis-2-benzyl-7-bromo-6-chloro-3a-methyl-3,3a,5,9b-tetrahydroisochromeno[3,4-c]pyrrol-1(2H)-one (230 mg, 19%) EI-MS: m/z=408.0 [M+H]⁺.

### 43.4 Preparation of trans-2-benzyl-7-bromo-6-chloro-3a-methyl-1,2,3,3a,5,9b-hexahydroisochromeno[3,4-c]pyrrole

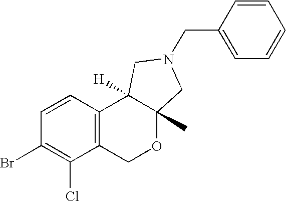

trans-2-Benzyl-7-bromo-6-chloro-3a-methyl-3,3a,5,9b-tetrahydroisochromeno[3,4-c]pyrrol-1(2H)-one (0.546 mmol, 0.222 g) was dissolved in tetrahydrofuran (10 ml) and to the resultant solution was added borane-DMS (3.82 mmol, 0.367 ml, 0.290 g). The mixture was refluxed under nitrogen 2.5 h. Further borane-DMS was added (0.37 ml) and the reaction mixture was again heated at reflux for a further 1 h. A third addition of borane/DMS (0.37 ml) was made and the reaction mixture was stirred at reflux for a further 1.5 hr. 5N HCl (5 ml) was added through the condenser and the reaction mixture was heated at reflux for 1.5 h. The resulting mixture was cooled to RT and the THF was removed under reduced pressure before addition of 4N NaOH to pH 14. The mixture was then extracted into EtOAc (2×10 mL) and combined organics extracts were then concentrated in vacuo to give a crude oil that was purified by silica column chromatography (4 g silica, eluting with DCM 2%-15% MeOH in DCM) to give trans-2-benzyl-7-bromo-6-chloro-3a-methyl-1,2,3,3a,5,9b-hexahydroisochromeno[3,4-c]pyrrole (150 mg, 70%), m/z=394.0 [M+H]⁺.

### 43.5 Preparation of trans-7-bromo-6-chloro-3a-methyl-1,2,3,3a,5,9b-hexahydroisochromeno[3,4-c]pyrrole hydrochloride

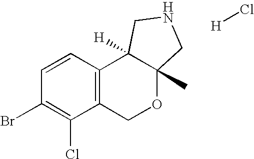

A solution of trans-2-benzyl-7-bromo-6-chloro-3a-methyl-1,2,3,3a,5,9b-hexahydroisochromeno[3,4-c]pyrrole (0.127 mmol, 0.05 g) and 1-chloroethyl chloroformate (0.637 mmol, 0.069 ml, 0.091 g) in toluene (1 ml) was subjected to microwave irradiation at 160° C. for 20 minutes. Methanol (0.4 ml) was added and the mixture was again subjected to microwave irradiation at 160° C. for 5 minutes. The MeOH was removed under reduced pressure causing precipitation of a white solid that was collected by filtration to give trans-7-bromo-6-chloro-3a-methyl-1,2,3,3a,5,9b-hexahydroisochromeno[3,4-c]pyrrole hydrochloride (25 mg, 58%), m/z=304.0 [M+H]⁺.

### EXAMPLE 44

### cis-7-Bromo-6-chloro-3a-methyl-1,2,3,3a,5,9b-hexahydroisochromeno[3,4-c]pyrrole hydrochloride

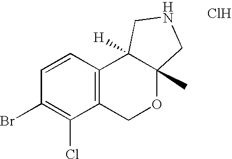

### 44.1 Preparation of cis-2-benzyl-7-bromo-6-chloro-3a-methyl-1,2,3,3a,5,9b-hexahydroisochromeno[3,4-c]pyrrole

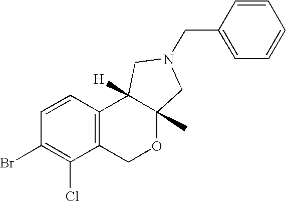

cis-2-Benzyl-7-bromo-6-chloro-3a-methyl-3,3a,5,9b-tetrahydroisochromeno[3,4-c]pyrrol-1(2H)-one (0.787 mmol, 0.32 g) was dissolved in tetrahydrofuran (10 ml) and to the resultant solution was added borane-DMS (5.51 mmol, 0.530 ml, 0.418 g). The reaction mixture was stirred at reflux in an oil bath at 70° C. for 3-4 h and then cooled to rt before adding 5N HCl (2 mL). The resulting mixture was again warmed to reflux for a further 1.5 h and then concentrated in vacuo to remove THF. The aqueous solution was basified to pH 14 by the addition of 4 N NaOH and then extracted with EtOAc (2×10 ml). The combined organic extracts were dried over Na₂SO₄ and concentrated in vacuo to give a crude yellow oil that was purified by silica column chromatography (12 g silica, eluting with neat DCM to 15% MeOH in DCM) to afford cis-2-benzyl-7-bromo-6-chloro-3a-methyl-1,2,3,3a,5,9b-hexahydroisochromeno[3,4-c]pyrrole (200 mg, 65%), m/z=394.0 [M+H]⁺.

### 44.2 Preparation of cis-7-bromo-6-chloro-3a-methyl-1,2,3,3a,5,9b-hexahydroisochromeno[3,4-c]pyrrole hydrochloride

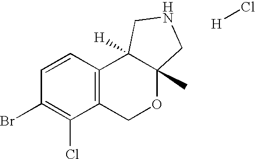

A solution of cis-2-benzyl-7-bromo-6-chloro-3a-methyl-1,2,3,3a,5,9b-hexahydroisochromeno[3,4-c]pyrrole (0.127 mmol, 0.05 g) and 1-chloroethyl chloroformate (0.637 mmol, 0.069 ml, 0.091 g) in toluene (1 ml) was subjected to microwave irradiation at 160° C. for 20 minutes. Methanol (0.4 ml) was then added and the mixture was again subjected to microwave irradiation at 160° C. for 5 minutes. The MeOH was removed under reduced pressure causing precipitation of a white solid that was collected by filtration to give cis-7-bromo-6-chloro-3a-methyl-1,2,3,3a,5,9b-hexahydroisochromeno[3,4-c]pyrrole hydrochloride (25 mg, 58%), m/z=304.0 [M+H]⁺.

### EXAMPLE 45

### cis-2-Benzyl-6-chloro-7-methoxy-3a-methyl-1,2,3,3a,5,9b-hexahydro isochromeno[3,4-c]pyrrole hydrochloride

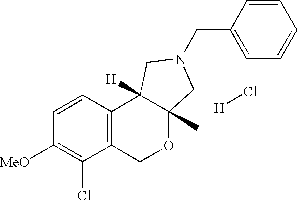

cis-2-Benzyl-7-bromo-6-chloro-3a-methyl-1,2,3,3a,5,9b-hexahydroisochromeno[3,4-c]pyrrole (0.178 mmol, 0.07 g) was treated with copper(I) bromide (0.071 mmol, 10.23 mg) and 25% sodium methoxide (4.37 mmol, 1 mL, 0.945 g) and ethyl acetate (0.1 mL). The mixture was irradiated in the microwave for 30 min at 120° C. The mixture was filtered through Dicalite then concentrated. Water (˜5 mL) was added and the product extracted with ethyl acetate (2×10 mL), dried (MgSO₄) and concentrated to afford a residue. Purification by basic prep-HPLC followed by HCl salt formation, using HCl in ether, afforded cis-2-benzyl-6-chloro-7-methoxy-3a-methyl-1,2,3,3a,5,9b-hexahydroisochromeno[3,4-c]pyrrole hydrochloride (96% yield), EI-MS: m/z=344.2 [M+H]⁺.

### EXAMPLE 46

### cis-2-Benzyl-6,7-dichloro-3a-methyl-1,2,3,3a,5,9b-hexahydroisochromeno[3,4-c]pyrrole

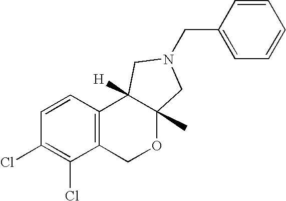

cis-2-Benzyl-7-bromo-6-chloro-3a-methyl-1,2,3,3a,5,9b-hexahydroisochromeno[3,4-c]pyrrole (0.178 mmol, 0.07 g) was dissolved in DMF (0.8 mL) and nickel(II) chloride (0.410 mmol, 0.053 g) added. The mixture was irradiated in the microwave at 200° C. for 30 minutes. Further nickel(II) chloride (0.410 mmol, 0.053 g) was added and the mixture irradiated for a further 30 min at 220° C. The solvent was removed under reduced pressure to give a crude residue which was chromatographed using a 4 g Redisep SiO₂ column eluting with ethyl acetate in heptane (5% to 50%), followed by methanol in ethyl acetate (2% to 10%) afforded cis-2-benzyl-6,7-dichloro-3a-methyl-1,2,3,3a,5,9b-hexahydroisochromeno[3,4-c]pyrrole (24% yield), EI-MS: m/z=348.2 [M+H]⁺.

### EXAMPLE 47

### cis-6-Chloro-7-methoxy-3a-methyl-1,2,3,3a,5,9b-hexahydroisochromeno[3,4-c]pyrrole hydrochloride

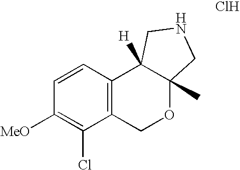

Similar procedures to that in Example 41 were employed to afford cis-6-chloro-7-methoxy-3a-methyl-1,2,3,3a,5,9b-hexahydroisochromeno[3,4-c]pyrrole hydrochloride, EI-MS: m/z=254.2 [M+H]⁺.

### EXAMPLE 48

### cis-6,7-Dichloro-3a-methyl-1,2,3,3a,5,9b-hexahydroisochromeno[3,4-c]pyrrole hydrochloride

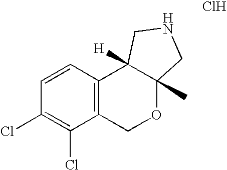

Similar procedures to that in Example 41 were employed to afford cis-6,7-dichloro-3a-methyl-1,2,3,3a,5,9b-hexahydroisochromeno[3,4-c]pyrrole hydrochloride, EI-MS: m/z=258.0 [M+H]⁺.

### EXAMPLE 49

### cis-7,8-Dichloro-2,3,4,4a,6,10b-hexahydro-1H-isochromeno[4,3-c]pyridine

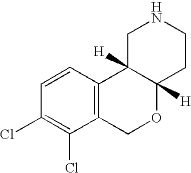

Similar procedures to that in Examples 46 and 41 were employed, using cis-2-benzyl-8-bromo-7-chloro-2,3,4,4a,6,10b-hexahydro-1H-isochromeno[4,3-c]pyridine to afford cis-7,8-dichloro-2,3,4,4a,6,10b-hexahydro-1H-isochromeno[4,3-c]pyridine, EI-MS: m/z=258.0, 260.0 [M+H]⁺.

### EXAMPLE 50

### trans-7,8-Dichloro-2,3,4,4a,6,10b-hexahydro-1H-isochromeno[4,3-c]pyridine

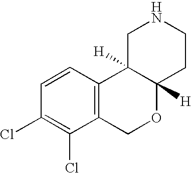

### 50.1 Preparation of trans-2-benzyl-7,8-dichloro-2,3,4,4a,6,10b-hexahydro-1H-isochromeno[4,3-c]pyridine

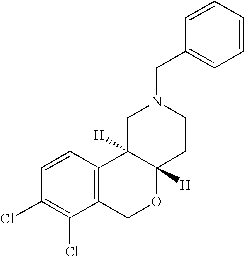

A mixture of trans-2-benzyl-8-bromo-7-chloro-2,3,4,4a,6,10b-hexahydro-1H-isochromeno[4,3-c]pyridine (0.178 mmol, 70 mg) and nickel (II) chloride (0.356 mmol, 47.1 mg) in NMP (1 ml) was heated in microwave at 210° C. for 0.5 h. Water was then added and the resulting mixture was extracted with DCM, then passed through SCX column (1 g) to give a brown solid that was purified by silica column chromatography (4 g silica, heptane:EtOAc=50:0, 50:5 to 50:10) to give trans-2-benzyl-7,8-dichloro-2,3,4,4a,6,10b-hexahydro-1H-isochromeno[4,3-c]pyridine (44 mg, 71%) as a pale yellow solid, EI-MS: m/z=348.1, 350.0 [M+H]⁺.

### 50.2 Preparation of trans-7,8-dichloro-2,3,4,4a,6,10b-hexahydro-1H-isochromeno[4,3-c]pyridine

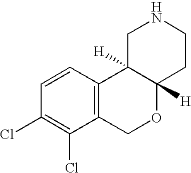

A solution of trans-2-benzyl-7,8-dichloro-2,3,4,4a,6,10b-hexahydro-1H-isochromeno[4,3-c]pyridine (0.100 mmol, 35 mg) and 1-chloroethyl chloroformate (0.506 mmol, 56 μl, 73.8 mg) in toluene (1 ml) was heated in a microwave reactor at 160° C. for 0.5 h. MeOH (0.5 ml) was added and the resulting mixture was heated further in a microwave at 160° C. for 5 min then passed through SCX column (1 g) to give an white solid that was purified by prep-LCMS (basic) to give trans-7,8-dichloro-2,3,4,4a,6,10b-hexahydro-1H-isochromeno[4,3-c]pyridine (14.0 mg, 54%) as a colourless solid, EI-MS: m/z=258.0, 260.0, 262.1 [M+H]⁺.

### EXAMPLE 51

### cis-7,10-Dichloro-2,3,4,4a,6,10b-hexahydro-1H-isochromeno[4,3-c]pyridine

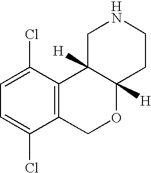

A mixture of cis-10-bromo-7-chloro-2,3,4,4a,6,10b-hexahydro-1H-isochromeno[4,3-c]pyridine (0.075 mmol, 22.7 mg) and nickel(II) chloride (0.150 mmol, 19.84 mg) in NMP (1 ml) was irradiated in the microwave at 210° C. for 0.5 h. Water was added and the mixture extracted with DCM, concentrated then passed through SCX column (1 g) to give 20 mg brown amorphous residue. The product was purified by basic prep-LCMS to afford cis-7,10-dichloro-2,3,4,4a,6,10b-hexahydro-1H-isochromeno[4,3-c]pyridine (26%), EI-MS: m/z=258.0, 260.0, 262.1 [M+H]⁺.

### EXAMPLE 52

### trans-7,10-Dichloro-2,3,4,4a,6,10b-hexahydro-1H-isochromeno[4,3-c]pyridine

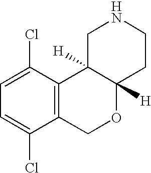

Similar procedures to that in example 51 were employed, using trans-10-bromo-7-chloro-2,3,4,4a,6,10b-hexahydro-1H-isochromeno[4,3-c]pyridine to afford trans-7,10-dichloro-2,3,4,4a,6,10b-hexahydro-1H-isochromeno[4,3-c]pyridine, EI-MS: m/z=258.0, 260.0, 262.1 [M+H]⁺.

### EXAMPLE 53

### cis-2-Benzyl-6-methyl-1,2,3,3a,5,9b-hexahydroisochromeno[3,4-c]pyrrole

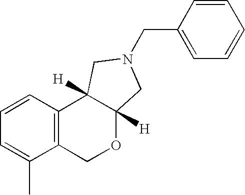

A mixture of cis-2-benzyl-6-chloro-3,3a,5,9b-tetrahydroisochromeno[3,4-c]pyrrol-1(2H)-one (0.300 mmol, 94 mg), nickel(II) bromide (1.498 mmol, 327 mg) in DMF (2 ml) was subjected to microwave irradiation at 200° C. for 20 minutes. The reaction mixture was partitioned between ethyl acetate and water. The organic phase was washed with brine, dried (Na₂SO₄) and concentrated in vacuo. Flash chromatography of the crude residue using ethyl acetate in heptane (20% to 40%) as the eluent afforded cis-2-benzyl-6-bromo-3,3a,5,9b-tetrahydroisochromeno[3,4-c]pyrrol-1(2H)-one (91.4 mg, 42.5% yield, containing cis-2-benzyl-3,3a,5,9b-tetrahydroisochromeno[3,4-c]pyrrol-1(2H)-one). The products were treated with trimethylboroxine (0.508 mmol, 0.071 mL, 63.8 mg), tetrakis(triphenylphosphine)palladium(0) (0.025 mmol, 29.4 mg) and potassium carbonate (0.508 mmol, 70.2 mg) in dioxane (1 mL) and subjected to microwave irradiation at 130° C. for 15 minutes. Water was added and the mixture extracted with ethyl acetate, washed with brine, dried (Na₂SO₄) and concentrated under reduced pressure. Flash chromatography of the crude residue using ethyl acetate in heptane (20% to 40%) afforded cis-2-benzyl-6-methyl-3,3a,5,9b-tetrahydroisochromeno[3,4-c]pyrrol-1(2H)-one (68 mg, containing cis-2-benzyl-3,3a,5,9b-tetrahydroisochromeno[3,4-c]pyrrol-1(2H)-one). The products were dissolved in tetrahydrofuran (6 ml) and to the resultant solution was added borane-dimethylsulfide complex (1.623 mmol, 0.156 ml). The mixture was refluxed under nitrogen for 3 h then cooled to 5° C. and 5N aqueous HCl (0.4 ml) added. The mixture was refluxed for an additional 1.5 h and then left to stand overnight (16 h). After addition of excess saturated aqueous NaHCO₃, the mixture was extracted with ethyl acetate, the organics were combined, washed with brine, dried (Na₂SO₄) and concentrated under reduced pressure to give a residue. Flash chromatography of the crude residue using ethyl acetate in heptane (30% to 100%) afforded cis-2-benzyl-6-methyl-1,2,3,3a,5,9b-hexahydroisochromeno[3,4-c]pyrrole (63 mg), EI-MS: m/z=280.1 [M+H]⁺.

### EXAMPLE 54

### cis-6-Methyl-1,2,3,3a,5,9b-hexahydroisochromeno[3,4-c]pyrrole hydrochloride

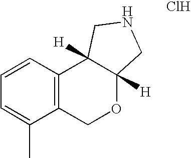

A similar reaction protocol to that in Example 30 was employed, using cis-2-benzyl-6-methyl-1,2,3,3a,5,9b-hexahydroisochromeno[3,4-c]pyrrole (0.109 mmol, 61 mg) to afford cis-6-methyl-1,2,3,3a,5,9b-hexahydroisochromeno[3,4-c]pyrrole hydrochloride (20%), EI-MS: m/z=190.6 [M+H]⁺.

### EXAMPLE 55

### cis-2-Benzyl-6-bromo-1,2,3,3a,5,9b-hexahydroisochromeno[3,4-c]pyrrole

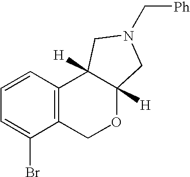

A similar reaction protocol to that in Example 1.9 was employed, using cis-2-benzyl-6-bromo-3,3a,5,9b-tetrahydroisochromeno[3,4-c]pyrrol-1(2H)-one (0.063 mmol, 22.6 mg) to afford cis-2-benzyl-6-bromo-1,2,3,3a,5,9b-hexahydroisochromeno[3,4-c]pyrrole (48% yield), EI-MS: m/z=346.1 [M+H]⁺.

### EXAMPLE 56

### cis-6-Bromo-1,2,3,3a,5,9b-hexahydroisochromeno[3,4-c]pyrrole hydrochloride

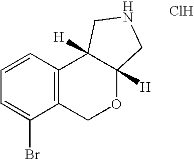

A similar reaction protocol to that in Example 30 was employed, using cis-2-benzyl-6-bromo-1,2,3,3a,5,9b-hexahydroisochromeno[3,4-c]pyrrole hydrochloride (0.025 mmol, 9.5 mg) to afford cis-6-bromo-1,2,3,3a,5,9b-hexahydroisochromeno[3,4-c]pyrrole hydrochloride (45% yield), EI-MS: m/z=254.1 [M+H]⁺.

### EXAMPLE 57

### cis-6-Chloro-7-methyl-1,2,3,3a,5,9b-hexahydroisochromeno[3,4-c]pyrrole hydrochloride

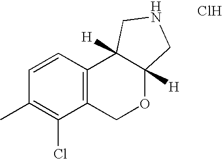

A mixture of cis-7-bromo-6-chloro-1,2,3,3a,5,9b-hexahydroisochromeno[3,4-c]pyrrole hydrochloride (0.025 mmol, 8 mg), trimethylboroxine (0.049 mmol, 6.88 μL, 6.18 mg), tetrakis(triphenylphosphine)palladium(0) (2.461 μmol, 2.84 mg) and potassium carbonate (0.098 mmol, 13.61 mg) in dioxane (1 mL) was subjected to microwave irradiation at 100° C. for 15 minutes, then at 120° C. for 15 minutes. The mixture was concentrated in vacuo then loaded onto a pre-acidified SCX column and eluted with 2M ammonia in methanol. The eluent was concentrated in vacuo and the residue purified by basic prep-HPLC. The desired fractions were concentrated in vacuo then converted to the HCl salt using HCl in ether to afford cis-6-chloro-7-methyl-1,2,3,3a,5,9b-hexahydroisochromeno[3,4-c]pyrrole hydrochloride (33% yield), EI-MS: m/z=224.1 [M+H]⁺.

### EXAMPLE 58

### cis-8-Bromo-6-(trifluoromethyl)-1,2,3,3a,5,9b-hexahydroisochromeno[3,4-c]pyrrole

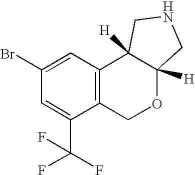

N-Bromosuccinimide (1.085 mmol, 193 mg) was added to a solution of cis-6-(trifluoromethyl)-1,2,3,3a,5,9b-hexahydroisochromeno[3,4-c]pyrrole (0.987 mmol, 240 mg) in concentrated sulphuric acid (2.4 ml) degassed with nitrogen. The reaction vessel was covered in tinfoil and the mixture stirred overnight (16 h) then poured onto ice. The mixture was basified with 4 M aqueous NaOH then extracted with ethyl acetate (×3), dried (Na₂SO₄) and concentrated in vacuo to afford a cis-8-bromo-6-(trifluoromethyl)-1,2,3,3a,5,9b-hexahydroisochromeno[3,4-c]pyrrole (98% yield), EI-MS: m/z=322.0 & 326.3 [M+H]⁺.

### EXAMPLE 59

### cis-8-Ethyl-6-(trifluoromethyl)-1,2,3,3a,5,9b-hexahydroisochromeno[3,4-c]pyrrole hydrochloride

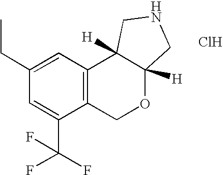

### 59.1 Preparation of cis-tert-butyl 8-bromo-6-(trifluoromethyl)-1,3,3a,9b-tetrahydroisochromeno[3,4-c]pyrrole-2(5H)-carboxylate

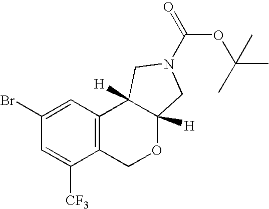

Di-tert-butyl dicarbonate (0.973 mmol, 0.212 g) was added to a suspension of cis-8-bromo-6-(trifluoromethyl)-1,2,3,3a,5,9b-hexahydroisochromeno[3,4-c]pyrrole (0.885 mmol, 0.285 g) and NaHCO₃ (5.31 mmol, 0.446 g) in methanol (7.56 ml). The mixture was sonicated for 2.5 hours during which time the temperature reached 40° C. Water was added and the mixture was extracted with ethyl acetate (×3), dried (Na₂SO₄) and concentrated in vacuo. Flash chromatography of the residue using ethyl acetate-heptane (2% to 20%) afforded cis-tert-butyl 8-bromo-6-(trifluoromethyl)-1,3,3a,9b-tetrahydroisochromeno[3,4-c]pyrrole-2(5H)-carboxylate (67% yield).

### 59.2 Preparation of cis-tert-butyl 6-(trifluoromethyl)-8-vinyl-1,3,3a,9b-tetrahydroisochromeno[3,4-c]pyrrole-2(5H)-carboxylate

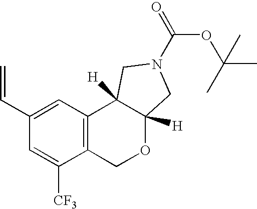

Tetrakis(triphenylphosphine)palladium(0) (7.39 μmol, 8.54 mg) was added in one portion to a degassed mixture of cis-tert-butyl 8-bromo-6-(trifluoromethyl)-1,3,3a,9b-tetrahydroisochromeno[3,4-c]pyrrole-2(5H)-carboxylate (0.308 mmol, 0.13 g), K₂CO₃ (0.364 mmol, 0.050 g) and 2,4,6-trivinylcyclotriboroxane pyridine complex (0.308 mmol, 0.074 g) in 1,2-dimethoxyethane (2.2 ml) and water (0.7 ml). The mixture was heated to 100° C. for 1.5 h, allowed to cool then diluted with brine and extracted with ethyl acetate (×3). The combined organics were dried (Na₂SO₄) and concentrated in vacuo. Flash chromatography of the residue using ethyl acetate in heptane (5% to 20%) gave cis-tert-butyl 6-(trifluoromethyl)-8-vinyl-1,3,3a,9b-tetrahydroisochromeno[3,4-c]pyrrole-2(5H)-carboxylate (97% yield).

### 59.3 Preparation of cis-tert-butyl 8-ethyl-6-(trifluoromethyl)-1,3,3a,9b-tetrahydroisochromeno[3,4-c]pyrrole-2(5H)-carboxylate

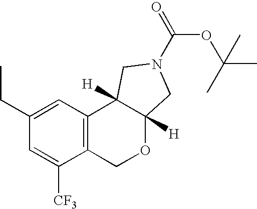

To a solution of nitrogen degassed cis-tert-butyl 6-(trifluoromethyl)-8-vinyl-1,3,3a,9b-tetrahydroisochromeno[3,4-c]pyrrole-2(5H)-carboxylate (0.225 mmol, 83 mg) in methanol (2 ml) was added 10% palladium on carbon (9.52 μmol, 10 mg). The mixture was stirred under a hydrogen balloon for 1.5 h then filtered through Dicalite® (which was flushed with EtOAc). The filtrate was concentrated in vacuo to afford cis-tert-butyl 8-ethyl-6-(trifluoromethyl)-1,3,3a,9b-tetrahydroisochromeno[3,4-c]pyrrole-2(5H)-carboxylate (96% yield).

### 59.4 Preparation of cis-8-ethyl-6-(trifluoromethyl)-1,2,3,3a,5,9b-hexahydroisochromeno[3,4-c]pyrrole hydrochloride

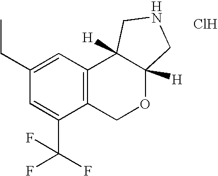

Trifluoroacetic acid (1 ml) was added to a solution of cis-tert-butyl 8-ethyl-6-(trifluoromethyl)-1,3,3a,9b-tetrahydroisochromeno[3,4-c]pyrrole-2(5H)-carboxylate (60 mg) in dichloromethane (2 ml) and the mixture was stirred for 1 h at rt. The mixture was concentrated in vacuo then loaded onto a pre-acidified SCX column. The column was flushed with excess methanol and then the product was eluted with 2M NH₃ in methanol. The eluent was concentrated in vacuo to afford the desired product and then HCl in ether added to make the HCl salt after concentration under reduced pressure to afford cis-8-ethyl-6-(trifluoromethyl)-1,2,3,3a,5,9b-hexahydroisochromeno[3,4-c]pyrrole hydrochloride (34% yield), EI-MS: m/z=272.5 [M+H]⁺.

### EXAMPLE 60

### cis-6-(Trifluoromethyl)-8-vinyl-1,2,3,3a,5,9b-hexahydroisochromeno[3,4-c]pyrrole hydrochloride

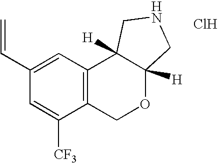

A similar reaction protocol to that in Example 59.4 was employed, using cis-tert-butyl 6-(trifluoromethyl)-8-vinyl-1,3,3a,9b-tetrahydroisochromeno[3,4-c]pyrrole-2(5H)-carboxylate (8 mg) to afford cis-6-(trifluoromethyl)-8-vinyl-1,2,3,3a,5,9b-hexahydroisochromeno[3,4-c]pyrrole hydrochloride (30% yield), EI-MS: m/z=270.5 [M+H]⁺.

### EXAMPLE 61

### cis-8-Propyl-6-(trifluoromethyl)-1,2,3,3a,5,9b-hexahydroisochromeno[3,4-c]pyrrole hydrochloride

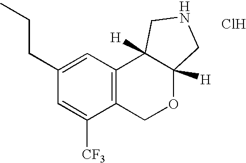

A similar reaction protocol to that in Example 59.2 to 59.4 was employed, using cis-propenylboronic acid to afford cis-8-propyl-6-(trifluoromethyl)-1,2,3,3a,5,9b-hexahydroisochromeno[3,4-c]pyrrole hydrochloride, EI-MS: m/z=285.8 [M+H]⁺.

### EXAMPLE 62

### cis-8-(3,5-Dimethylisoxazol-4-yl)-6-(trifluoromethyl)-1,2,3,3a,5,9b-hexahydroisochromeno[3,4-c]pyrrole

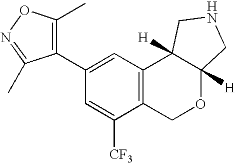

A similar reaction protocol to that in Example 59.2 and 59.4 was employed, using 3,5-dimethylisoxazol-4-ylboronic acid to afford cis-8-(3,5-dimethylisoxazol-4-yl)-6-(trifluoromethyl)-1,2,3,3a,5,9b-hexahydroisochromeno[3,4-c]pyrrole, EI-MS: m/z=339.0 [M+H]⁺.

### EXAMPLE 63

### cis-8-Cyclopropyl-6-(trifluoromethyl)-1,2,3,3a,5,9b-hexahydroisochromeno[3,4-c]pyrrole hydrochloride

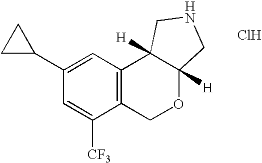

A similar reaction protocol to that in Example 59.2 and 59.4 was employed, using cyclopropylboronic acid to afford cis-8-cyclopropyl-6-(trifluoromethyl)-1,2,3,3a,5,9b-hexahydroisochromeno[3,4-c]pyrrole hydrochloride, EI-MS: m/z=283.9 [M+H]⁺.

### EXAMPLE 64

### cis-8-Isopropyl-6-(trifluoromethyl)-1,2,3,3a,5,9b-hexahydroisochromeno[3,4-c]pyrrole hydrochloride

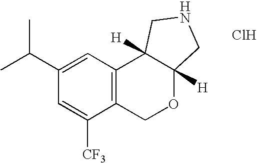

A similar reaction protocol to that in Example 59.2 to 59.4 was employed, using 2-isopropenyl-4,4,5,5-tetramethyl-1,3,2-dioxaborolane to afford cis-8-isopropyl-6-(trifluoromethyl)-1,2,3,3a,5,9b-hexahydroisochromeno[3,4-c]pyrrole hydrochloride, EI-MS: m/z=285.9 [M+H]⁺.

### EXAMPLE 65

### cis-6-Chloro-3a-methyl-7-(prop-1-en-2-yl)-1,2,3,3a,5,9b-hexahydroisochromeno[3,4-c]pyrrole hydrochloride

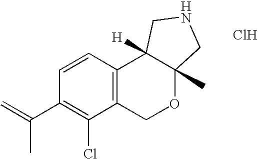

A similar reaction protocol to that in Example 59.2 was employed, using 2-isopropenyl-4,4,5,5-tetramethyl-1,3,2-dioxaborolane and cis-2-benzyl-7-bromo-6-chloro-3a-methyl-1,2,3,3a,5,9b-hexahydroisochromeno[3,4-c]pyrrole to afford cis-2-benzyl-6-chloro-3a-methyl-7-(prop-1-en-2-yl)-1,2,3,3a,5,9b-hexahydroisochromeno[3,4-c]pyrrole. A solution of cis-2-benzyl-6-chloro-3a-methyl-7-(prop-1-en-2-yl)-1,2,3,3a,5,9b-hexahydroisochromeno[3,4-c]pyrrole (0.127 mmol, 45 mg) in dry toluene (1.5 mL) was treated with 1-chloroethyl chloroformate (0.636 mmol, 0.069 ml, 91 mg) and subjected to microwave irradiation at 160° C. for 20 minutes. Methanol (0.4 mL) was added and the mixture subjected to microwave irradiation at 160° C. for 5 minutes. The mixture was concentrated under reduced pressure to afford a residue. The solid product was triturated with ether, filtered giving cis-6-chloro-3a-methyl-7-(prop-1-en-2-yl)-1,2,3,3a,5,9b-hexahydroisochromeno[3,4-c]pyrrole hydrochloride (19 mg), EI-MS: m/z=264.2 [M+H]⁺.

### EXAMPLE 66

### cis-8-Methyl-6-(trifluoromethyl)-1,2,3,3a,5,9b-hexahydroisochromeno[3,4-c]pyrrole hydrochloride

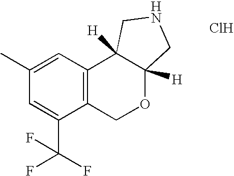

### 66.1 Preparation of cis-tert-butyl 8-methyl-6-(trifluoromethyl)-1,3,3a,9b-tetrahydroisochromeno[3,4-c]pyrrole-2(5H)-carboxylate

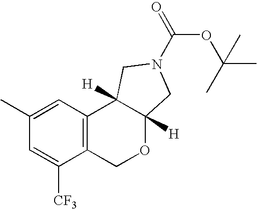

cis-tert-Butyl 8-bromo-6-(trifluoromethyl)-1,3,3a,9b-tetrahydroisochromeno[3,4-c]pyrrole-2(5H)-carboxylate (0.166 mmol, 70 mg), trimethylboroxine (0.332 mmol, 0.046 mL), tetrakis(triphenylphosphine)palladium(0) (0.017 mmol, 19.16 mg) and potassium carbonate (0.332 mmol, 45.8 mg) in dioxane (2 mL) was subjected to microwave irradiation at 120° C. for 20 minutes. Water was added and the mixture was extracted with ethyl acetate, dried (Na₂SO₄) and concentrated in vacuo. Flash chromatography of the residue using ethyl acetate in heptane (5% to 20%) as the eluent to afford cis-tert-butyl 8-methyl-6-(trifluoromethyl)-1,3,3a,9b-tetrahydroisochromeno[3,4-c]pyrrole-2(5H)-carboxylate (98% yield).

### 66.2 Preparation of cis-8-methyl-6-(trifluoromethyl)-1,2,3,3a,5,9b-hexahydroisochromeno[3,4-c]pyrrole hydrochloride

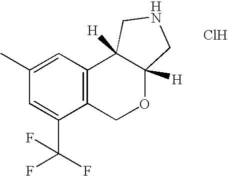

A similar reaction protocol to that in Example 59.4 was employed, using cis-tert-butyl 8-methyl-6-(trifluoromethyl)-1,3,3a,9b-tetrahydroisochromeno[3,4-c]pyrrole-2(5H)-carboxylate (0.112 mmol, 40 mg) to afford cis-8-methyl-6-(trifluoromethyl)-1,2,3,3a,5,9b-hexahydroisochromeno[3,4-c]pyrrole hydrochloride (76% yield), EI-MS: m/z=257.9 [M+H]⁺.

### EXAMPLE 67

### cis-8-Phenyl-6-(trifluoromethyl)-1,2,3,3a,5,9b-hexahydroisochromeno[3,4-c]pyrrole hydrochloride

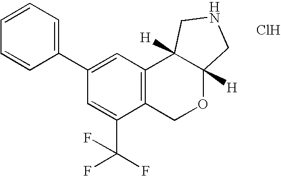

### 67.1 Preparation of cis-tert-butyl 8-phenyl-6-(trifluoromethyl)-1,3,3a,9b-tetrahydroisochromeno[3,4-c]pyrrole-2(5H)-carboxylate

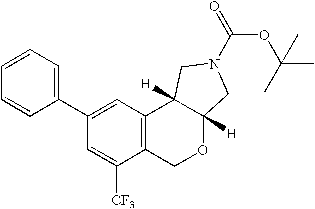

cis-tert-Butyl 8-bromo-6-(trifluoromethyl)-1,3,3a,9b-tetrahydroisochromeno[3,4-c]pyrrole-2(5H)-carboxylate (0.142 mmol, 60 mg), phenylboronic acid (0.284 mmol, 34.7 mg), ethanol (1.5 ml), DME (1.5 ml) and sodium carbonate (0.968 mmol, 0.484 ml) was placed into a microwave vail to which tetrakis(triphenylphosphine)Pd(0) (2.84 μmol, 3.28 mg) was added. The mixture was heated in a microwave at 120° C. for 30 min. The reaction mixture was quenched into 5 N sodium hydroxide solution, extracted with ethyl acetate, dried (MgSO₄) and concentrated in vacuo. Flash chromatography of the residue using ethyl acetate in heptane as the eluent gave cis-tert-butyl 8-phenyl-6-(trifluoromethyl)-1,3,3a,9b-tetrahydroisochromeno[3,4-c]pyrrole-2(5H)-carboxylate (91% yield)

### 67.2 Preparation of cis-8-phenyl-6-(trifluoromethyl)-1,2,3,3a,5,9b-hexahydroisochromeno[3,4-c]pyrrole hydrochloride

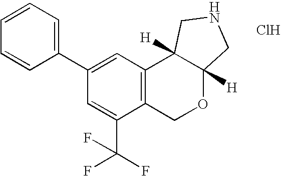

A similar reaction protocol to that in Example 59.4 was employed, using cis-tert-butyl 8-phenyl-6-(trifluoromethyl)-1,3,3a,9b-tetrahydroisochromeno[3,4-c]pyrrole-2(5H)-carboxylate (0.129 mmol, 54 mg) to afford cis-8-phenyl-6-(trifluoromethyl)-1,2,3,3a,5,9b-hexahydroisochromeno[3,4-c]pyrrole hydrochloride (45.8% yield), EI-MS: m/z=320.0 [M+H]⁺.

### EXAMPLE 68

### cis-8-Chloro-6-(trifluoromethyl)-1,2,3,3a,5,9b-hexahydroisochromeno[3,4-c]pyrrole hydrochloride

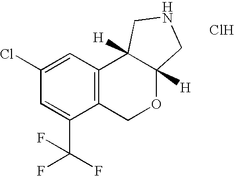

A mixture of cis-8-bromo-6-(trifluoromethyl)-1,2,3,3a,5,9b-hexahydroisochromeno[3,4-c]pyrrole (0.093 mmol, 30 mg) and nickel (II) chloride (0.373 mmol, 48.3 mg) in N-methyl-2-pyrrolidinone (2 mL) was subjected to microwave irradiation at 210° C. for 30 minutes. Reaction mixture was partitioned between ethyl acetate and 2M NaOH. The organic phase was dried (Na₂SO₄) and concentrated to give crude residue. The crude residue was purified on an ion exchange column (SCX, 0.5 g) and then purified by basic prep-HPLC. The resultant product was concentrated then converted to the HCl salt by dissolving in DCM and adding HCl in ether to afford cis-8-chloro-6-(trifluoromethyl)-1,2,3,3a,5,9b-hexahydroisochromeno[3,4-c]pyrrole hydrochloride (15.72% yield), EI-MS: m/z=278.3 [M+H]⁺.

### EXAMPLE 69

### cis-N,N-Dimethyl-6-(trifluoromethyl)-1,2,3,3a,5,9b-hexahydroisochromeno[3,4-c]pyrrol-8-amine hydrochloride

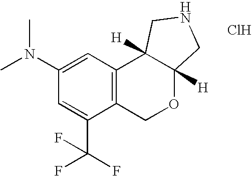

cis-tert-Butyl 8-bromo-6-(trifluoromethyl)-1,3,3a,9b-tetrahydroisochromeno[3,4-c]pyrrole-2(5H)-carboxylate (0.071 mmol, 30 mg), dimethylamine (2 M in THF, 0.107 mmol, 0.053 mL), sodium tert-butoxide (0.107 mmol, 10.24 mg), (R)-(+)-2,2′-bis(diphenylphosphino)-1,1′-binaphthyl (7.10 μmol, 4.42 mg), tris(dibenzylideneacetone)dipalladium (0) (3.55 μmol, 3.25 mg) and toluene (0.5 mL) were added to a microwave vial and irradiated for 15 minutes at 135° C. The mixture was partitioned between ethyl acetate and water. The aqueous layer was extracted with ethyl acetate and the combined organic layers were washed with brine, dried (Na₂SO₄) and concentrated under reduced pressure to give a residue. HCl (5N, 0.5 mL) was added to the residue in dioxane (1 mL) and methanol (0.5 mL). The mixture was stirred at 100° C. for 0.5 h and then concentrated to afford a residue which was treated with SCX, then purified with basic prep-HPLC to afford pure product. The product was conveted to the HCl salt by dissolving in DCM and adding HCl in ether before concentrating to dryness to afford cis-N,N-dimethyl-6-(trifluoromethyl)-1,2,3,3a,5,9b-hexahydroisochromeno[3,4-c]pyrrol-8-amine hydrochloride (9% yield), EI-MS: m/z=287.0 [M+H]⁺.

### EXAMPLE 70

### cis-2-Benzyl-7-chloro-2,3,4,4a,6,10b-hexahydro-1H-isochromeno[4,3-c]pyridine & trans-2-benzyl-7-chloro-2,3,4,4a,6,10b-hexahydro-1H-isochromeno[4,3-c]pyridine

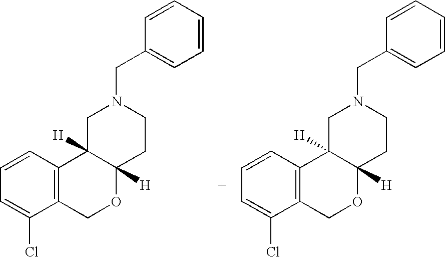

### 70.1 Preparation of 3-(benzylamino)propan-1-ol

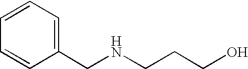

To a stirred mixture of 3-aminopropan-1-ol (10.00 mmol, 751 mg) and benzaldehyde (10 mmol, 1061 mg) in dichloromethane (35 ml) was added sodium triacetoxyborohydride (14.00 mmol, 2967 mg). The reaction was stirred overnight then quenched with methanol (200 ml) and product isolated by SCX (eluting with 7N ammonia in MeOH). Evaporation gave 0.85 g of 3-(benzylamino)propan-1-ol (51% yield), EI-MS: m/z=166.4 [M+H]⁺.

### 70.2 Preparation of N-benzyl-3-chloro-N-(3-hydroxypropyl)-1,2-dihydrocyclobutabenzene-1-carboxamide

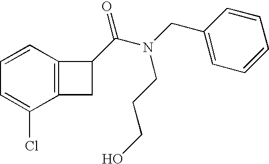

A mixture of 3-chloro-1,2-dihydrocyclobutabenzene-1-carboxylic acid (5.14 mmol, 0.939 g), 1-hydroxybezotriazole hydrate (5.14 mmol, 0.788 g) and N, N′-methanediylidenedipropan-2-amine (5.14 mmol, 0.649 g) in N-methylpyrrolidinone (15 ml) was stirred for 30 minutes. A solution of 3-(benzylamino)propan-1-ol (5.14 mmol, 0.85 g) in N-methylpyrrolidinone (5 ml) was then added and the reaction stirred over the weekend. Aqueous work up (acid/base) followed by flash chromatography using ethyl acetate in heptane (50%) as the eluent afforded N-benzyl-3-chloro-N-(3-hydroxypropyl)-1,2-dihydrocyclobutabenzene-1-carboxamide (33% yield), EI-MS: m/z=330.3 [M+H]⁺.

### 70.3 Preparation of cis-2-benzyl-7-chloro-2,3,4,4a,6,10b-hexahydro-1H-isochromeno[4,3-c]pyridine & trans-2-benzyl-7-chloro-2,3,4,4a,6,10b-hexahydro-1H-isochromeno[4,3-c]pyridine

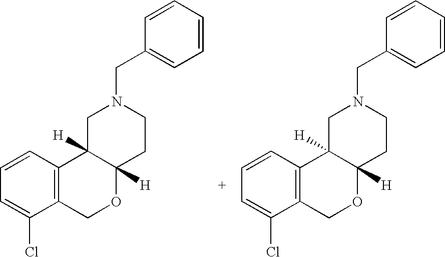

N-Benzyl-3-chloro-N-(3-hydroxypropyl)-1,2-dihydrocyclobutabenzene-1-carboxamide (1.698 mmol, 0.56 g) in dichloromethane (20 ml) was treated with Dess-Martin reagent (4 ml, 15% w/w in dichloromethane) for 2 hr at room temp. The aldehyde product was isolated by flash chromatography using ethyl acetate in heptane (50%) as the eluent to afford the desired aldehyde (300 mg). This was dissolved in 1,4-dioxane (5 ml) and heated in the microwave at 210° C. for 60 minutes. Twenty percent of the final solution was removed and purified by prep-HPLC to isolate the cis- and trans-amide products and the remaining material was evaporated. The remaining crude cis/trans-amide products were dissolved in tetrahydrofuran (20 ml) and treated with borane dimethylsulfide complex (1 ml). The mixture was heated at reflux overnight. After cooling 5N HCl (4 ml) was added and reflux continued for 2 h. The solution was then cooled, diluted with MeOH (300 ml) and purified by SCX (20 g column) chromatography. The basic products were then eluted with 7N ammonia in MeOH and evaporated to yield 200 mg colourless glass. LC-MS analysis showed 2 close running peaks of the desired mass. Purification by prep-HPLC (pH10, Xbridge column, 30 minute linear gradient) allowed separation and isolation to afford trans-2-benzyl-7-chloro-2,3,4,4a,6,10b-hexahydro-1H-isochromeno[4,3-c]pyridine (45 mg), EI-MS: m/z=314.4 [M+H]⁺, followed by cis-2-benzyl-7-chloro-2,3,4,4a,6,10b-hexahydro-1H-isochromeno[4,3-c]pyridine (16 mg), EI-MS: m/z=314.4 [M+H]⁺.

### EXAMPLE 71

### cis-7-Chloro-2,3,4,4a,6,10b-hexahydro-1H-isochromeno[4,3-c]pyridine

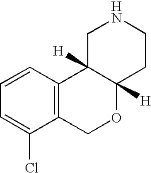

A similar reaction protocol to that in Example 30 was employed, using cis-2-benzyl-7-chloro-2,3,4,4a,6,10b-hexahydro-1H-isochromeno[4,3-c]pyridine (40 mg) to afford cis-7-chloro-2,3,4,4a,6,10b-hexahydro-1H-isochromeno[4,3-c]pyridine (11 mg), EI-MS: m/z=224.3 [M+H]⁺.

### EXAMPLE 72

### trans-7-Chloro-2,3,4,4a,6,10b-hexahydro-1H-isochromeno[4,3-c]pyridine

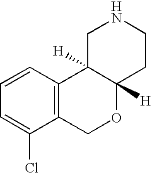

A similar reaction protocol to that in Example 30 was employed, using trans-2-benzyl-7-chloro-2,3,4,4a,6,10b-hexahydro-1H-isochromeno[4,3-c]pyridine (13 mg) to afford trans-7-chloro-2,3,4,4a,6,10b-hexahydro-1H-isochromeno[4,3-c]pyridine (2 mg), EI-MS: m/z=224.3 [M+H]⁺.

### EXAMPLE 73

### cis-2-Benzyl-8-bromo-7-chloro-2,3,4,4a,6,10b-hexahydro-1H-isochromeno[4,3-c]pyridine

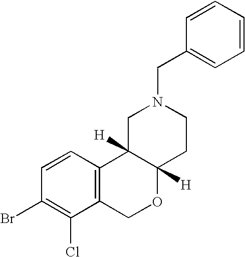

A similar reaction protocol to that in Example 70.2 and 70.3 was employed, using 4-bromo-3-chloro-1,2-dihydrocyclobutabenzene-1-carboxylic acid to afford cis-2-benzyl-8-bromo-7-chloro-2,3,4,4a,6,10b-hexahydro-1H-isochromeno[4,3-c]pyridine, EI-MS: m/z=392.0, 393.9, 396.0 [M+H]⁺.

### EXAMPLE 74

### trans-2-Benzyl-8-bromo-7-chloro-2,3,4,4a,6,10b-hexahydro-1H-isochromeno[4,3-c]pyridine

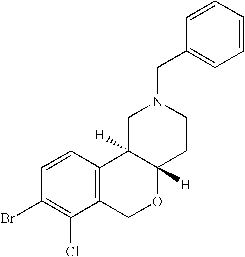

A similar reaction protocol to that in Example 70.2 and 70.3 was employed, using 4-bromo-3-chloro-1,2-dihydrocyclobutabenzene-1-carboxylic acid to afford trans-2-benzyl-8-bromo-7-chloro-2,3,4,4a,6,10b-hexahydro-1H-isochromeno[4,3-c]pyridine, EI-MS: m/z=391.9, 394.0, 395.9 [M+H]⁺.

### EXAMPLE 75

### trans-8-Bromo-7-chloro-2,3,4,4a,6,10b-hexahydro-1H-isochromeno[4,3-c]pyridine

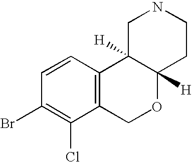

A similar reaction protocol to that in Example 30 was employed, using trans-2-benzyl-8-bromo-7-chloro-2,3,4,4a,6,10b-hexahydro-1H-isochromeno[4,3-c]pyridine to afford trans-8-bromo-7-chloro-2,3,4,4a,6,10b-hexahydro-1H-isochromeno[4,3-c]pyridine, EI-MS: m/z=302.0, 304.0, 306.0 [M+H]⁺.

### EXAMPLE 76

### cis-8-Bromo-7-chloro-2,3,4,4a,6,10b-hexahydro-1H-isochromeno[4,3-c]pyridine

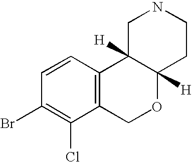

A similar reaction protocol to that in Example 30 was employed, using cis-2-benzyl-8-bromo-7-chloro-2,3,4,4a,6,10b-hexahydro-1H-isochromeno[4,3-c]pyridine to afford cis-8-bromo-7-chloro-2,3,4,4a,6,10b-hexahydro-1H-isochromeno[4,3-c]pyridine, EI-MS: m/z=302.1, 304.0, 306.0 [M+H]⁺.

### EXAMPLE 77

### cis-10-Bromo-7-chloro-2,3,4,4a,6,10b-hexahydro-1H-isochromeno[4,3-c]pyridine

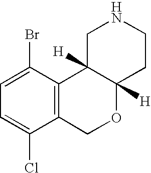

N-Bromosuccinimide (0.409 mmol, 73.6 mg) was added to a solution of cis-7-chloro-2,3,4,4a,6,10b-hexahydro-1H-isochromeno[4,3-c]pyridine (0.409 mmol, 91.6 mg) in sulfuric acid (1 ml) at 20° C. and stirred for 21 h. Water and DCM were added and the phases separated. The aqueous phase was made alkaline with 4 N NaOH and the mixture extracted with DCM. The combined organics were dried (Na₂SO₄) and concentrated under reduced pressure to give a residue. The residue was flash chromatographed, using DCM:MeOH:ammonia (50:0:0, 50:2:0, 45:3:0, 50:5:0 then 50:4.5:0.5). The resultant product was further purified by basic prep-HPLC to afford cis-10-bromo-7-chloro-2,3,4,4a,6,10b-hexahydro-1H-isochromeno[4,3-c]pyridine (43% yield), EI-MS: m/z=302.0, 304.0, 306.0 [M+H]⁺.

### EXAMPLE 78

### trans-10-Bromo-7-chloro-2,3,4,4a,6,10b-hexahydro-1H-isochromeno[4,3-c]pyridine

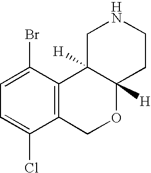

A similar reaction protocol to that in Example 77 was employed, using trans-7-chloro-2,3,4,4a,6,10b-hexahydro-1H-isochromeno[4,3-c]pyridine to afford trans-10-bromo-7-chloro-2,3,4,4a,6,10b-hexahydro-1H-isochromeno[4,3-c]pyridine, EI-MS: m/z=302.0, 304.0, 306.0 [M+H]⁺.

### EXAMPLE 79

### trans-7-Chloro-4-a-methyl-2,3,4,4a,6,10b-hexahydro-1H-isochromeno[4,3-c]pyridine

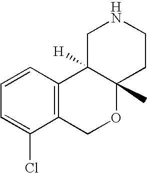

### 79.1 Preparation of 4-(benzylamino)butan-2-one hydrochloride

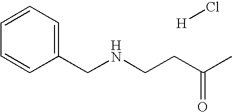

A mixture of benzylamine hydrochloride (34.5 mmol, 5.01 g), acetone (173 mmol, 12.79 ml, 10.13 g) and formaldehyde (34.5 mmol, 2.59 ml, 2.80 g) was refluxed at 75° C. for 18 h. The mixture was concentrated under reduced pressure then recrystallized from acetone to afford 4-(benzylamino)butan-2-one hydrochloride (68%).

### 79.2 Preparation of N-benzyl-3-chloro-N-(3-oxobutyl)-1,2-dihydro cyclobutabenzene-1-carboxamide

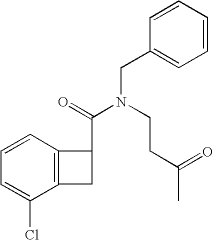

Cyclophos (5.62 mmol, 3.34 ml, 3.57 g) was added to a solution of 3-chloro-1,2-dihydrocyclobutabenzene-1-carboxylic acid (5.62 mmol, 1.025 g), 4-(benzylamino)butan-2-one hydrochloride (3.74 mmol, 1 g) and triethylamine (11.23 mmol, 1.581 ml, 1.148 g) in DCM (20 ml). The mixture was stirred at 20° C. for 5 h then water and DCM added. The phases were mixed then separated and concentrated under reduced pressure. The residue was purified by flash chromatography, eluting with ethyl acetate in heptane (0 to 50%) to afford N-benzyl-3-chloro-N-(3-oxobutyl)-1,2-dihydrocyclobutabenzene-1-carboxamide (77% yield).

### 79.3 Preparation of trans-7-chloro-4-a-methyl-2,3,4,4a,6,10b-hexahydro-1H-isochromeno[4,3-c]pyridine

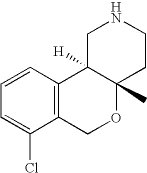

A similar reaction protocol to that in Example 70.3 and 72 was employed, using N-benzyl-3-chloro-N-(3-oxobutyl)-1,2-dihydrocyclobutabenzene-1-carboxamide to afford trans-7-chloro-4-a-methyl-2,3,4,4a,6,10b-hexahydro-1H-isochromeno[4,3-c]pyridine, EI-MS: m/z=238.3, 240.0 [M+H]⁺.

### EXAMPLE 80

### cis-6-Bromo-3a-methyl-1,2,3,3a,5,9b-hexahydroisochromeno[3,4-c]pyrrole hydrochloride

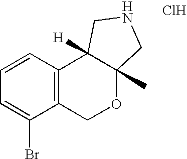

### 80.1 Preparation of 2,6-dibromobenzaldehyde

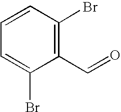

1,3-Dibromobenzene (424 mmol, 100 g) in dry THF (1 L) was cooled to −78° C. and lithium diisopropylamide (509 mmol, 254 ml) added dropwise. After addition, the mixture was stirred for 30 min at −78° C. and then N,N-dimethylformamide (509 mmol, 39.4 ml, 37.2 g). The mixture was stirred for a further 30 min and then 5 N HCl (300 ml) was added. The mixture was warmed to room temperature and the phases separated. The aqueous phase was extracted with diethyl ether (2×250 ml). Combined organics were washed with brine (300 ml), dried (MgSO₄) and concentrated in vacuo to afford a brown oil. The product was precipitated using heptane and stirring overnight to afford 2,6-dibromobenzaldehyde (62.5 g).

### 80.2 Preparation of 3-bromo-1,2-dihydrocyclobutabenzene-1-carboxylic acid

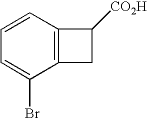

Using 2,6-dibromobenzaldehyde, the 3-bromo-1,2-dihydrocyclobutabenzene-1-carboxylic acid was prepared using similar procedures as those in Example 1.1 to 1.5.

### 80.3 Preparation of cis-6-bromo-3a-methyl-1,2,3,3a,5,9b-hexahydroisochromeno[3,4-c]pyrrole hydrochloride

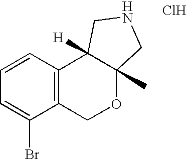

Similar protocols to procedures in Example 40 and 41 were employed, using 3-bromo-1,2-dihydrocyclobutabenzene-1-carboxylic acid to afford cis-6-bromo-3a-methyl-1,2,3,3a,5,9b-hexahydroisochromeno[3,4-c]pyrrole hydrochloride, EI-MS: m/z=270.0 [M+H]⁺.

### EXAMPLE 81

### trans-6-Bromo-3a-methyl-1,2,3,3a,5,9b-hexahydroisochromeno[3,4-c]pyrrole hydrochloride

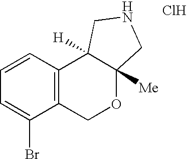

Using 3-bromo-1,2-dihydrocyclobutabenzene-1-carboxylic acid, the procedures in examples 40 and 41 were employed to afford cis-6-bromo-3a-methyl-1,2,3,3a,5,9b-hexahydroisochromeno[3,4-c]pyrrole hydrochloride, EI-MS: m/z=270.4 and 271.5 [M+H]⁺.

### EXAMPLE 82

### cis-6-Chloro-3a-(trifluoromethyl)-1,2,3,3a,5,9b-hexahydroisochromeno[3,4-c]pyrrole

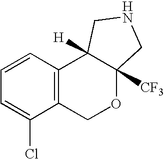

Similar protocols to procedures in Example 40 and 41 were employed, using 3-chloro-1,2-dihydrocyclobutabenzene-1-carboxylic acid and 1-benzylamino-3,3,3-trifluoropropan-2-ol to afford cis-6-chloro-3a-(trifluoromethyl)-1,2,3,3a,5,9b-hexahydroisochromeno[3,4-c]pyrrole, EI-MS: m/z=278.0 [M+H]⁺.

### EXAMPLE 83

### cis-2-Benzyl-6-chloro-3a-ethyl-1,2,3,3a,5,9b-hexahydroisochromeno[3,4-c]pyrrole hydrochloride

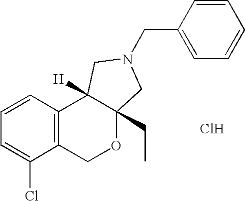

### 83.1 Preparation of 1-(benzylamino)butan-2-ol

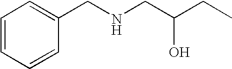

To a solution of the 2-ethyloxirane (41.6 mmol, 3 g) and benzylamine (41.6 mmol, 4.46 g) in acetonitrile (125 ml) was added calcium trifluoromethanesulfonate (20.80 mmol, 7.04 g) and the reaction was stirred at room temperature for 5 h and then concentrated under reduced pressure. The mixture was acidified with dilute HCl and extracted with ethyl acetate. The acidic aqueous phase was made basic with NaOH and extracted with ethyl acetate (×2), dried (Na₂SO₄) and concentrated under reduced pressure to afford 1-(benzylamino)butan-2-ol (6.8 g)

### 83.2 Preparation of cis-2-benzyl-6-chloro-3a-ethyl-1,2,3,3a,5,9b-hexahydroisochromeno[3,4-c]pyrrole hydrochloride

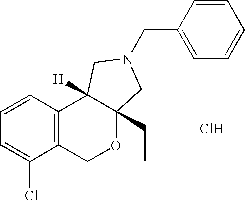

Similar protocols to procedures in Example 40 were employed, using 1-(benzylamino)butan-2-ol to afford cis-2-benzyl-6-chloro-3a-ethyl-1,2,3,3a,5,9b-hexahydroisochromeno[3,4-c]pyrrole hydrochloride, EI-MS: m/z=328.2 [M+H]⁺.

### EXAMPLE 84

### trans-6-Chloro-3a-ethyl-1,2,3,3a,5,9b-hexahydroisochromeno[3,4-c]pyrrole hydrochloride

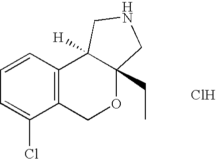

Similar protocols to procedures in Example 40 and 41 were employed, using 1-(benzylamino)butan-2-ol to afford trans-6-chloro-3a-ethyl-1,2,3,3a,5,9b-hexahydroisochromeno[3,4-c]pyrrole hydrochloride, EI-MS: m/z=238.0 [M+H]⁺.

### EXAMPLE 85

### cis-6-Chloro-3a-ethyl-1,2,3,3a,5,9b-hexahydroisochromeno[3,4-c]pyrrole hydrochloride

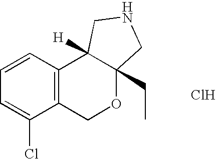

Similar protocols to procedures in Example 41 were employed, using cis-2-benzyl-6-chloro-3a-ethyl-1,2,3,3a,5,9b-hexahydroisochromeno[3,4-c]pyrrole to afford cis-6-chloro-3a-ethyl-1,2,3,3a,5,9b-hexahydroisochromeno[3,4-c]pyrrole hydrochloride, EI-MS: m/z=238.0 [M+H]⁺.

### EXAMPLE 86

### trans-6-chloro-3a-(fluoromethyl)-1,2,3,3a,5,9b-hexahydroisochromeno[3,4-c]pyrrole 2,2,2-trifluoroacetate

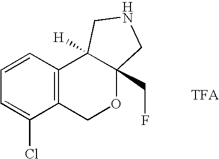

### 86.1 Preparation of 1-(benzylamino)-3-fluoropropan-2-ol

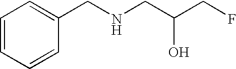

To a solution of the 2-(fluoromethyl)oxirane (39.4 mmol, 3 g) and benzylamine (31.5 mmol, 3.38 g) in acetonitrile (118 ml) was added calcium trifluoromethanesulfonate (19.72 mmol, 6.67 g) and the reaction was stirred at room temperature for 1 h, then at 50° C. for 4 h. The mixture was concentrated under reduced pressure, dilute HCl was added and the mixture was extracted with ethyl acetate. The acidic aqueous phase was then made basic with NaOH and extracted with ethyl acetate (×2), dried (Na₂SO₄) and concentrated under reduced pressure to afford 1-(benzylamino)-3-fluoropropan-2-ol (37% yield).

### 86.2 Preparation of trans-6-chloro-3a-(fluoromethyl)-1,2,3,3a,5,9b-hexahydroisochromeno[3,4-c]pyrrole 2,2,2-trifluoroacetate

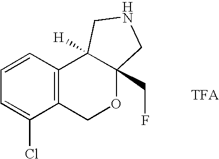

Similar protocols to procedures in Example 40 and 41 were employed, using 1-(benzylamino)-3-fluoropropan-2-ol to afford trans-6-chloro-3a-(fluoromethyl)-1,2,3,3a,5,9b-hexahydroisochromeno[3,4-c]pyrrole 2,2,2-trifluoroacetate, EI-MS: m/z=242.0 [M+H]⁺.

### EXAMPLE 87

### cis-6-Chloro-3a-(fluoromethyl)-1,2,3,3a,5,9b-hexahydroisochromeno[3,4-c]pyrrole hydrochloride

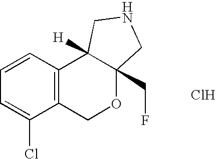

Similar protocols to procedures in Example 40 and 41 were employed, using 1-(benzylamino)-3-fluoropropan-2-ol to afford cis-6-chloro-3a-(fluoromethyl)-1,2,3,3a,5,9b-hexahydroisochromeno[3,4-c]pyrrole hydrochloride, EI-MS: m/z=242.0 [M+H]⁺.

### EXAMPLE 88

### cis-9-Bromo-6-chloro-1,2,3,3a,5,9b-hexahydroisochromeno[3,4-c]pyrrole

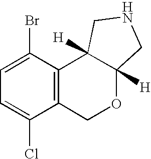

N-Bromosuccinimide (0.954 mmol, 170 mg) was added to a solution of cis-6-chloro-1,2,3,3a,5,9b-hexahydroisochromeno[3,4-c]pyrrole (0.954 mmol, 200 mg) in sulfuric acid (1 ml) and the mixture was stirred at room temperature for 16 h in the dark. The reaction mixture was poured into ice-water (15 ml) and washed with ether. The aqueous layer was basified with 4N NaOH and extracted with ether. The combined organic extracts were washed with brine, dried (Na₂SO₄) and concentrated in vacuo to afford cis-9-bromo-6-chloro-1,2,3,3a,5,9b-hexahydroisochromeno[3,4-c]pyrrole (159.3 mg), EI-MS: m/z=290.1 [M+H]⁺.

### EXAMPLE 89

### cis-2-Methyl-6-(trifluoromethyl)-1,2,3,3a,5,9b-hexahydroisochromeno[3,4-c]pyrrole hydrochloride

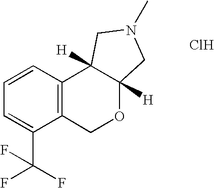

To a microwave vial was added cis-6-(trifluoromethyl)-1,2,3,3a,5,9b-hexahydroisochromeno[3,4-c]pyrrole (25 mg, 0.103 mmol), DMF (1 mL), formaldehyde (0.514 mmol) and glacial acetic acid (50 μL). Sodium triacetoxyborohydride (0.514 mmol) was then added to vial. The reaction was irradiated in the microwave at 100° C. for 10 min. The reaction was quenched with water (1 mL), diluted with MeOH and loaded onto a pre-acidified 2 g SCX cartridge, eluting the crude products with 2 M NH₃ in MeOH to afford a crude residue that was purified by prep-LCMS (basic modifier). The purified product was then concentrated and converted to the HCl salt by treatment with HCl in ether (0.5 mL). The product was concentrated to afford cis-2-methyl-6-(trifluoromethyl)-1,2,3,3a,5,9b-hexahydroisochromeno[3,4-c]pyrrole hydrochloride (10.4 mg, 34%), EI-MS: m/z=258.1 [M+H]⁺.

The above reaction was employed using the appropriate aldehydes and ketones to afford the following products.

### EXAMPLE 90

cis-2-Ethyl-6-(trifluoromethyl)-1,2,3,3a,5,9b-hexahydroisochromeno[3,4-c]pyrrole hydrochloride (6.5 mg, 20%), EI-MS: m/z=272.5 [M+H]⁺.

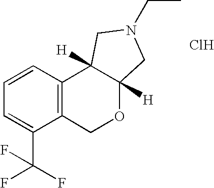

### EXAMPLE 91

cis-2-Isopropyl-6-(trifluoromethyl)-1,2,3,3a,5,9b-hexahydroisochromeno[3,4-c]pyrrole hydrochloride (7.7 mg, 23%), EI-MS: m/z=285.9 [M+H]⁺.

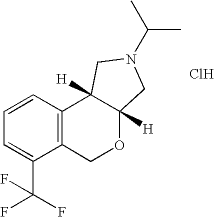

### EXAMPLE 92

cis-2-Propyl-6-(trifluoromethyl)-1,2,3,3a,5,9b-hexahydroisochromeno[3,4-c]pyrrole hydrochloride (8 mg, 24%), EI-MS: m/z=285.9 [M+H]⁺.

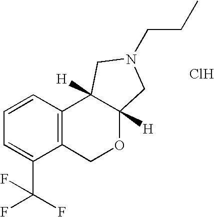

### EXAMPLE 93

cis-2-Isobutyl-6-(trifluoromethyl)-1,2,3,3a,5,9b-hexahydroisochromeno[3,4-c]pyrrole hydrochloride (12 mg, 35%), EI-MS: m/z=300.0 [M+H]⁺.

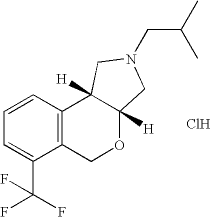

### EXAMPLE 94

cis-2-Cyclobutyl-6-(trifluoromethyl)-1,2,3,3a,5,9b-hexahydroisochromeno[3,4-c]pyrrole hydrochloride (6 mg, 17%), EI-MS: m/z=298.3 [M+H]⁺.

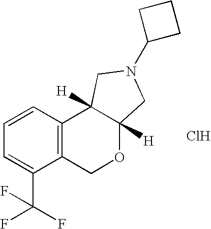

### EXAMPLE 95

cis-2-Cyclopentyl-6-(trifluoromethyl)-1,2,3,3a,5,9b-hexahydroisochromeno[3,4-c]pyrrole hydrochloride (10 mg, 28%), EI-MS: m/z=311.8 [M+H]⁺.

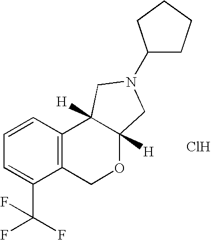

### EXAMPLE 96

cis-2-Benzyl-6-(trifluoromethyl)-1,2,3,3a,5,9b-hexahydroisochromeno[3,4-c]pyrrole hydrochloride (19 mg, 47%), EI-MS: m/z=368.0 [M+H]⁺.

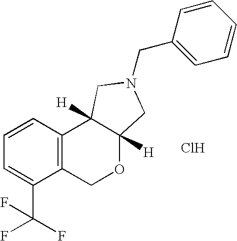

### EXAMPLE 97

### trans-6-Cyclopentyl-1,2,3,3a,5,9b-hexahydroisochromeno[3,4-c]pyrrole 2,2,2-trifluoroacetate

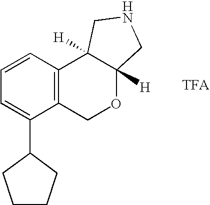

### 97.1 Preparation of trans-tert-butyl 6-(trifluoromethylsulfonyloxy)-1,3,3a,9b-tetrahydroisochromeno[3,4-c]pyrrole-2(5H)-carboxylate

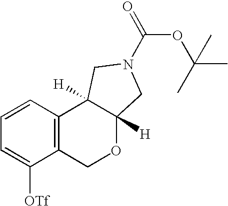

To a solution of trans-tert-butyl 6-hydroxy-1,3,3a,9b-tetrahydroisochromeno[3,4-c]pyrrole-2(5H)-carboxylate (1.613 mmol, 470 mg) in THF (13.4 ml) was added sodium hydride (1.936 mmol, 77 mg) followed by a solution of N-phenyltrifluoromethanesulfonimide (1.775 mmol, 634 mg) in THF (2 ml). The resultant mixture was stirred at room temperature for 24 hours then the THF was removed and the residue partitioned between DCM and saturated aqueous NaHCO₃. The organic extracts was washed with brine, dried (Na₂SO₄) and concentrated under reduced pressure to afford trans-tert-butyl 6-(trifluoromethylsulfonyloxy)-1,3,3a,9b-tetrahydroisochromeno[3,4-c]pyrrole-2(5H)-carboxylate (102% yield) which was used without further purification.

### 97.2 Preparation of trans-tert-butyl 6-cyclopentenyl-1,3,3a,9b-tetrahydroisochromeno[3,4-c]pyrrole-2(5H)-carboxylate

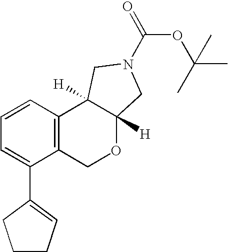

Tetrakis(triphenylphosphine)Pd(0) (0.018 mmol, 20.47 mg) was added in one portion to a mixture of trans-tert-butyl 6-(trifluoromethylsulfonyloxy)-1,3,3a,9b-tetrahydroisochromeno[3,4-c]pyrrole-2(5H)-carboxylate (0.354 mmol, 150 mg), cyclopentenylboronic acid (0.425 mmol, 47.6 mg) and potassium carbonate (0.531 mmol, 73.4 mg) in 1,4-dioxane (5 ml)/water (0.5 ml) mixture. The mixture was subjected to microwave irradiation at 130° C. for 30 minutes. The reaction mixture was partitioned between ethyl acetate and water, and the aqueous layer was extracted with ethyl acetate. The combined organic extracts were washed with brine, dried (Na₂SO₄) and concentrated under reduced pressure. The crude residue was purified by silica column chromatography (eluting with ethyl acetate in heptane, 0 to 20%) to afford trans-tert-butyl 6-cyclopentenyl-1,3,3a,9b-tetrahydroisochromeno[3,4-c]pyrrole-2(5H)-carboxylate (78% yield).

### 97.3 Preparation of trans-tert-butyl 6-cyclopentyl-1,3,3a,9b-tetrahydro isochromeno[3,4-c]pyrrole-2(5H)-carboxylate

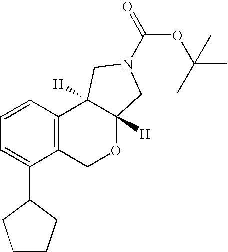

A mixture of trans-tert-butyl 6-cyclopentenyl-1,3,3a,9b-tetrahydroisochromeno[3,4-c]pyrrole-2(5H)-carboxylate (0.275 mmol, 94 mg) and 10% palladium on carbon (0.014 mmol, 14.65 mg) in ethanol (8097 μl) was stirred vigorously at room temperature under an atmosphere of hydrogen (balloon) for 24 hour. The spent catalyst was removed by filtration through celite and the resulting filtrate was concentrated in vacuo to afford a residue that was purified by silica column chromatography (eluting with ethyl acetate in heptane, 0 to 10%) to afford trans-tert-butyl 6-cyclopentyl-1,3,3a,9b-tetrahydroisochromeno[3,4-c]pyrrole-2(5H)-carboxylate (70% yield).

### 97.4 Preparation of trans-6-cyclopentyl-1,2,3,3a,5,9b-hexahydroisochromeno[3,4-c]pyrrole 2,2,2-trifluoroacetate

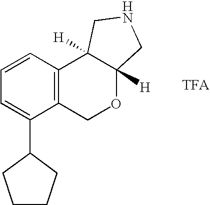

cis-tert-Butyl 6-cyclopentyl-1,3,3a,9b-tetrahydroisochromeno[3,4-c]pyrrole-2(5H)-carboxylate (0.192 mmol, 66 mg) was dissolved in ethyl acetate (20 ml) and then hydrogen chloride (10 mmol, 365 mg) gas was bubbled through the mixture. The solution was stirred for 3 h then concentrated and purified by acidic prep-HPLC to afford trans-6-cyclopentyl-1,2,3,3a,5,9b-hexahydroisochromeno[3,4-c]pyrrole 2,2,2-trifluoroacetate (47% yield), EI-MS: m/z=244.1 [M+H]⁺.

### EXAMPLE 98

### trans-6-Isopropyl-1,2,3,3a,5,9b-hexahydroisochromeno[3,4-c]pyrrole 2,2,2-trifluoroacetate

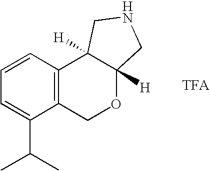

Similar protocols to procedures in Example 97 were employed, using 2-isopropenyl-4,4,5,5-tetramethyl-1,3,2,dioxaborolane and trans-tert-butyl 6-(trifluoromethylsulfonyloxy)-1,3,3a,9b-tetrahydroisochromeno[3,4-c]pyrrole-2(5H)-carboxylate to afford trans-6-isopropyl-1,2,3,3a,5,9b-hexahydroisochromeno[3,4-c]pyrrole 2,2,2-trifluoroacetate, EI-MS: m/z=218.6 [M+H]⁺.

### EXAMPLE 99

### cis-6-Isopropyl-1,2,3,3a,5,9b-hexahydroisochromeno[3,4-c]pyrrole hydrochloride

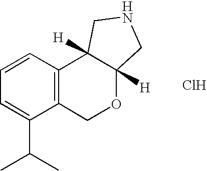

The procedures described in example 97 were employed, using 2-isopropenyl-4,4,5,5-tetramethyl-1,3,2,dioxaborolane and cis-tert-butyl 6-(trifluoromethylsulfonyloxy)-1,3,3a,9b-tetrahydroisochromeno[3,4-c]pyrrole-2(5H)-carboxylate to afford cis-6-isopropyl-1,2,3,3a,5,9b-hexahydroisochromeno[3,4-c]pyrrole hydrochloride, EI-MS: m/z=218.4 [M+H]⁺.

### EXAMPLE 100

### trans-6-Isopropyl-3a-methyl-1,2,3,3a,5,9b-hexahydroisochromeno[3,4-c]pyrrole 2,2,2-trifluoroacetate

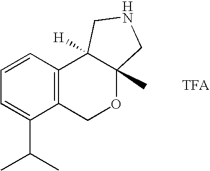

The procedures described in example 97 were employed, using 4,4,5,5-tetramethyl-2-(prop-1-en-2-yl)-1,3,2-dioxaborolane and trans-tert-butyl 3a-methyl-6-(trifluoromethylsulfonyloxy)-1,3,3a,9b-tetrahydroisochromeno[3,4-c]pyrrole-2(5H)-carboxylate to afford trans-6-isopropyl-3a-methyl-1,2,3,3a,5,9b-hexahydroisochromeno[3,4-c]pyrrole 2,2,2-trifluoroacetate, EI-MS: m/z=232.4 [M+H]⁺.

### EXAMPLE 101

### cis-6-Isopropyl-3a-methyl-1,2,3,3a,5,9b-hexahydroisochromeno[3,4-c]pyrrole hydrochloride

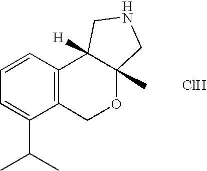

The procedures described in example 97 were employed, using 2-isopropenyl-4,4,5,5-tetramethyl-1,3,2,dioxaborolane and cis-tert-butyl 3a-methyl-6-(trifluoromethylsulfonyloxy)-1,3,3a,9b-tetrahydroisochromeno[3,4-c]pyrrole-2(5H)-carboxylate to afford cis-6-isopropyl-3a-methyl-1,2,3,3a,5,9b-hexahydroisochromeno[3,4-c]pyrrole hydrochloride, EI-MS: m/z=232.4 [M+H]⁺.

### EXAMPLE 102

### cis-6-Propyl-1,2,3,3a,5,9b-hexahydroisochromeno[3,4-c]pyrrole hydrochloride

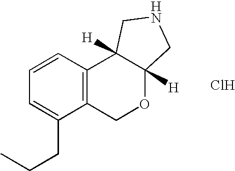

The procedures described in example 97 were employed, using (Z)-prop-1-enylboronic acid and cis-tert-butyl 6-(trifluoromethylsulfonyloxy)-1,3,3a,9b-tetrahydroisochromeno[3,4-c]pyrrole-2(5H)-carboxylate to afford cis-6-propyl-1,2,3,3a,5,9b-hexahydroisochromeno[3,4-c]pyrrole hydrochloride, EI-MS: m/z=218.6 [M+H]⁺.

### EXAMPLE 103

### cis-3a-Methyl-6-propyl-1,2,3,3a,5,9b-hexahydroisochromeno[3,4-c]pyrrole 2,2,2-trifluoroacetate

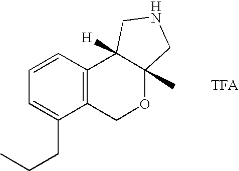

The procedures described in Example 97 were employed, using (Z)-prop-1-enylboronic acid and cis-tert-butyl 3a-methyl-6-(trifluoromethylsulfonyloxy)-1,3,3a,9b-tetrahydroisochromeno[3,4-c]pyrrole-2(5H)-carboxylate to afford cis-3a-methyl-6-propyl-1,2,3,3a,5,9b-hexahydroisochromeno[3,4-c]pyrrole 2,2,2-trifluoroacetate, EI-MS: m/z=232.4 [M+H]⁺.

### EXAMPLE 104

### cis-6-Cyclopentyl-3a-methyl-1,2,3,3a,5,9b-hexahydroisochromeno[3,4-c]pyrrole 2,2,2-trifluoroacetate

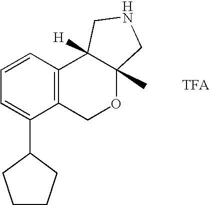

The procedures described in Example 97 were employed, using cis-tert-butyl 3a-methyl-6-(trifluoromethylsulfonyloxy)-1,3,3a,9b-tetrahydroisochromeno[3,4-c]pyrrole-2(5H)-carboxylate and cyclopentenylboronic acid to afford cis-6-cyclopentyl-3a-methyl-1,2,3,3a,5,9b-hexahydroisochromeno[3,4-c]pyrrole 2,2,2-trifluoroacetate, EI-MS: m/z=258.5 [M+H]⁺.

### EXAMPLE 105

### cis-3a,6-Dimethyl-1,2,3,3a,5,9b-hexahydroisochromeno[3,4-c]pyrrole 2,2,2-trifluoroacetate

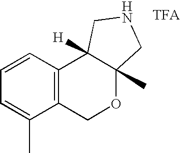

The procedures described in Example 97 were employed, using cis-tert-butyl 3a-methyl-6-(trifluoromethylsulfonyloxy)-1,3,3a,9b-tetrahydroisochromeno[3,4-c]pyrrole-2(5H)-carboxylate and trimethylboroxine to afford cis-3a,6-dimethyl-1,2,3,3a,5,9b-hexahydroisochromeno[3,4-c]pyrrole 2,2,2-trifluoroacetate, EI-MS: m/z=204.3 [M+H]⁺.

### EXAMPLE 106

### trans-3a,6-Dimethyl-1,2,3,3a,5,9b-hexahydroisochromeno[3,4-c]pyrrole 2,2,2-trifluoroacetate

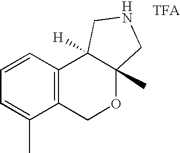

The procedures described in Example 97 were employed, using trans-tert-butyl 3a-methyl-6-(trifluoromethylsulfonyloxy)-1,3,3a,9b-tetrahydroisochromeno[3,4-c]pyrrole-2(5H)-carboxylate and trimethylboroxine to afford trans-3a,6-dimethyl-1,2,3,3a,5,9b-hexahydroisochromeno[3,4-c]pyrrole 2,2,2-trifluoroacetate, EI-MS: m/z=204.3 [M+H]⁺.

### EXAMPLE 107

### trans-6-Ethyl-3a-methyl-1,2,3,3a,5,9b-hexahydroisochromeno[3,4-c]pyrrole 2,2,2-trifluoroacetate

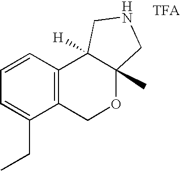

Similar procedures to those described in Example 97 were employed, using trans-tert-butyl 3a-methyl-6-(trifluoromethylsulfonyloxy)-1,3,3a,9b-tetrahydroisochromeno[3,4-c]pyrrole-2(5H)-carboxylate and 4,6-trivinylcyclotriboroxane pyridine complex to afford trans-6-ethyl-3a-methyl-1,2,3,3a,5,9b-hexahydroisochromeno[3,4-c]pyrrole 2,2,2-trifluoroacetate, EI-MS: m/z=218.4 [M+H]⁺.

### EXAMPLE 108

### cis-6-Isobutyl-3a-methyl-1,2,3,3a,5,9b-hexahydroisochromeno[3,4-c]pyrrole 2,2,2-trifluoroacetate

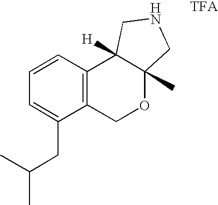

Similar procedures to those described in Example 97 were employed, using cis-tert-butyl 3a-methyl-6-(trifluoromethylsulfonyloxy)-1,3,3a,9b-tetrahydroisochromeno[3,4-c]pyrrole-2(5H)-carboxylate and 2-methylprop-1-enylboronic acid to afford cis-6-isobutyl-3a-methyl-1,2,3,3a,5,9b-hexahydroisochromeno[3,4-c]pyrrole 2,2,2-trifluoroacetate, EI-MS: m/z=246.4 [M+H]⁺.

### EXAMPLE 109

### cis-6-(3,5-Dimethylisoxazol-4-yl)-3a-methyl-1,2,3,3a,5,9b-hexahydroisochromeno[3,4-c]pyrrole 2,2,2-trifluoroacetate

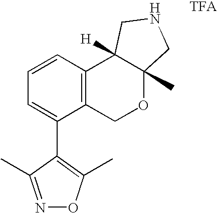

Similar procedures to those described in Example 97 were employed, using cis-tert-butyl 3a-methyl-6-(trifluoromethylsulfonyloxy)-1,3,3a,9b-tetrahydroisochromeno[3,4-c]pyrrole-2(5H)-carboxylate and 3,5-dimethylisoxazol-4-ylboronic acid to afford cis-6-(3,5-dimethylisoxazol-4-yl)-3a-methyl-1,2,3,3a,5,9b-hexahydroisochromeno[3,4-c]pyrrole 2,2,2-trifluoroacetate, EI-MS: m/z=285.3 [M+H]⁺.

### EXAMPLE 110

### cis-6-(Isoxazol-4-yl)-1,2,3,3a,5,9b-hexahydroisochromeno[3,4-c]pyrrole hydrochloride

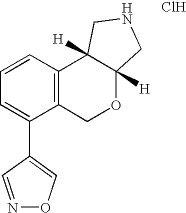

Similar procedures to those described in Example 97 were employed, using cis-tert-butyl 6-(trifluoromethylsulfonyloxy)-1,3,3a,9b-tetrahydroisochromeno[3,4-c]pyrrole-2(5H)-carboxylate and isoxazol-4-ylboronic acid to afford cis-6-(isoxazol-4-yl)-1,2,3,3a,5,9b-hexahydroisochromeno[3,4-c]pyrrole hydrochloride, EI-MS: m/z=243.8 [M+H]⁺.

### EXAMPLE 111

### cis-6-Phenyl-1,2,3,3a,5,9b-hexahydroisochromeno[3,4-c]pyrrole hydrochloride

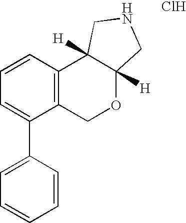

### 111.1 Preparation of cis-2-benzyl-6-phenyl-1,2,3,3a,5,9b-hexahydroisochromeno[3,4-c]pyrrole

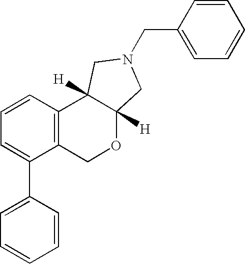

Tetrakis(triphenylphosphine)Pd(0) (2.179 μmol, 2.52 mg) was added in one portion to a mixture of cis-2-benzyl-6-bromo-1,2,3,3a,5,9b-hexahydroisochromeno[3,4-c]pyrrole (0.044 mmol, 15 mg), phenylboronic acid (0.052 mmol, 6.38 mg) and potassium carbonate (0.065 mmol, 9.03 mg) in 1,4-dioxane (1 mL) and water (0.2 mL) mixture. The mixture was subjected to microwave irradiation at 130° C. for 15 minutes, then passed through an SCX cartridge to afford a residue that was purified by basic prep-HPLC to afford cis-2-benzyl-6-phenyl-1,2,3,3a,5,9b-hexahydroisochromeno[3,4-c]pyrrole (83% yield), EI-MS: m/z=342.0 [M+H]⁺.

### 111.2 Preparation of cis-6-phenyl-1,2,3,3a,5,9b-hexahydroisochromeno[3,4-c]pyrrole hydrochloride

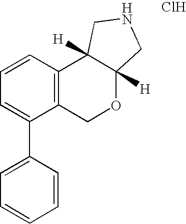

A solution of cis-2-benzyl-6-phenyl-1,2,3,3a,5,9b-hexahydroisochromeno[3,4-c]pyrrole (0.028 mmol, 9.5 mg) and 1-chloroethyl chloroformate (0.139 mmol, 0.015 ml, 19.89 mg) in toluene (2 ml) was subjected to microwave irradiation at 150° C. for 20 minutes, then methanol (0.5 ml) was added to the mixture and the mixture was subjected to microwave irradiation at 150° C. for 5 minutes. After addition of excess saturated aqueous NaHCO₃ (10 ml), the mixture was extracted with ethyl acetate (10 ml, ×3). The combined organic extracts were washed with brine, dried (Na₂SO₄) and concentrated under reduced pressure to give a residue. The crude residue was purified by basic prep-HPLC (basic modifier) and converted to the HCl salt to afford cis-6-phenyl-1,2,3,3a,5,9b-hexahydroisochromeno[3,4-c]pyrrole hydrochloride (38% yield), EI-MS: m/z=252.4 [M+H]⁺.

### EXAMPLE 112

### cis-6-(2-Methoxyphenyl)-1,2,3,3a,5,9b-hexahydroisochromeno[3,4-c]pyrrole hydrochloride

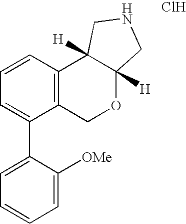

Similar procedures described in Example 97 were employed, using cis-tert-butyl 6-(trifluoromethylsulfonyloxy)-1,3,3a,9b-tetrahydroisochromeno[3,4-c]pyrrole-2(5H)-carboxylate and 2-methoxyphenylboronic acid to afford cis-6-(2-methoxyphenyl)-1,2,3,3a,5,9b-hexahydroisochromeno[3,4-c]pyrrole hydrochloride, EI-MS: m/z=282.3 [M+H]⁺.

### EXAMPLE 113

### cis-6-(2-Fluorophenyl)-1,2,3,3a,5,9b-hexahydroisochromeno[3,4-c]pyrrole hydrochloride

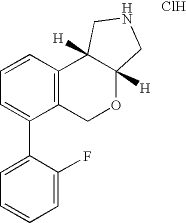

Similar procedures described in Example 97 were employed, using cis-tert-butyl 6-(trifluoromethylsulfonyloxy)-1,3,3a,9b-tetrahydroisochromeno[3,4-c]pyrrole-2(5H)-carboxylate and 2-fluorophenylboronic acid to afford cis-6-(2-fluorophenyl)-1,2,3,3a,5,9b-hexahydroisochromeno[3,4-c]pyrrole hydrochloride, EI-MS: m/z=270.5 [M+H]⁺.

### EXAMPLE 114

### cis-6-Ethyl-1,2,3,3a,5,9b-hexahydroisochromeno[3,4-c]pyrrole

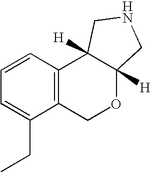

Similar procedures described in Example 97 were employed, using cis-tert-butyl 6-(trifluoromethylsulfonyloxy)-1,3,3a,9b-tetrahydroisochromeno[3,4-c]pyrrole-2(5H)-carboxylate and 2,4,6-trivinylcyclotriboroxane pyridine complex to afford cis-6-ethyl-1,2,3,3a,5,9b-hexahydroisochromeno[3,4-c]pyrrole, EI-MS: m/z=204.4 [M+H]⁺.

### EXAMPLE 115

### cis-N,N-Dimethyl-1,2,3,3a,5,9b-hexahydroisochromeno[3,4-c]pyrrol-6-amine

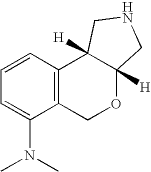

### 115.1 Preparation of cis-tert-butyl 6-(dimethylamino)-1,3,3a,9b-tetrahydroisochromeno[3,4-c]pyrrole-2(5H)-carboxylate

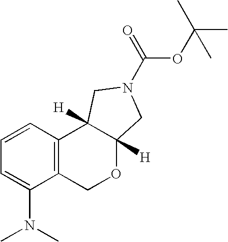

cis-tert-Butyl 6-(trifluoromethylsulfonyloxy)-1,3,3a,9b-tetrahydroisochromeno[3,4-c]pyrrole-2(5H)-carboxylate (0.035 mmol, 15 mg), dimethylamine in THF (0.053 mmol, 0.027 mL), sodium t-butoxide (0.053 mmol, 5.11 mg), BINAP (3.54 μmol, 2.206 mg), tris(dibenzylideneacetone)dipalladium(0) (1.771 μmol, 1.622 mg) and toluene (0.5 mL) were added to a microwave vial and irradiated for 15 minutes at 135° C. The mixture was partitioned between ethyl acetate and water. The aqueous layer was extracted with further ethyl acetate and the combined organic extracts were washed with brine, dried (Na₂SO₄) and concentrated under reduced pressure to give a residue. The residue was purified by prep-HPLC to afford cis-tert-butyl 6-(dimethylamino)-1,3,3a,9b-tetrahydroisochromeno[3,4-c]pyrrole-2(5H)-carboxylate (76% yield).

### 115.2 Preparation of cis-N,N-dimethyl-1,2,3,3a,5,9b-hexahydroisochromeno[3,4-c]pyrrol-6-amine

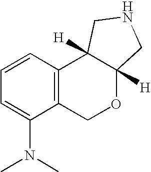

5 N HCl (0.075 ml) was added to a solution of cis-tert-butyl 6-(dimethylamino)-1,3,3a,9b-tetrahydroisochromeno[3,4-c]pyrrole-2(5H)-carboxylate (7.54 μmol, 2.4 mg) in dioxane (1.0 ml)/MeOH (0.2 .ml). The mixture was stirred at 100° C. for 0.5 h. The solvent was removed under vacuum and the residue was passed through an SCX cartridge and then purified by basic prep-HPLC to afford cis-N,N-dimethyl-1,2,3,3a,5,9b-hexahydroisochromeno[3,4-c]pyrrol-6-amine (67% yield), EI-MS: m/z=219.4 [M+H]⁺.

### EXAMPLE 116

### cis-3a-Methyl-6-(pyrrolidin-1-yl)-1,2,3,3a,5,9b-hexahydroisochromeno[3,4-c]pyrrole 2,2,2-trifluoroacetate

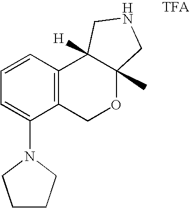

Similar procedures described in Example 115 were employed, using cis-tert-butyl 3a-methyl-6-(trifluoromethylsulfonyloxy)-1,3,3a,9b-tetrahydroisochromeno[3,4-c]pyrrole-2(5H)-carboxylate and pyrrolidine to afford cis-3a-methyl-6-(pyrrolidin-1-yl)-1,2,3,3a,5,9b-hexahydroisochromeno[3,4-c]pyrrole 2,2,2-trifluoroacetate, EI-MS: m/z=259.3 [M+H]⁺.

### EXAMPLE 117

### cis-6-Cyclopropyl-1,2,3,3a,5,9b-hexahydroisochromeno[3,4-c]pyrrole

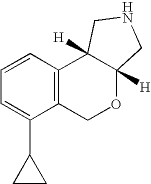

### cis-6-cyclopropyl-1,3,3a,9b-tetrahydroisochromeno[3,4-c]pyrrole-2(5H)-carboxylate

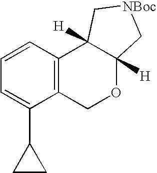

1,1′-Bis(diphenylphosphino)ferrocenedichloropalladium(II) (1.771 μmol, 1.296 mg) was added in one portion to a mixture of cis-tert-butyl 6-(trifluoromethylsulfonyloxy)-1,3,3a,9b-tetrahydroisochromeno[3,4-c]pyrrole-2(5H)-carboxylate (0.035 mmol, 15 mg), cyclopropylboronic acid (0.053 mmol, 4.56 mg) and potassium carbonate (0.053 mmol, 7.34 mg) in a solution of 1,4-dioxane (1.480 mL)/water (0.296 mL). The mixture was irradiated in the microwave at 130° C. for 20 minutes. Further 1,1′-bis(diphenylphosphino)ferrocenedichloropalladium(II) (1.771 μmol, 1.296 mg) and cyclopropylboronic acid (0.053 mmol, 4.56 mg) were added and the mixture was irradiated at 150° C. for 20 minutes. The above was repeated and the mixture was irradiated at 160° C. for a further 40 minutes. The mixture was then partitioned between water (10 mL) and ethyl acetate (10 mL). The aqueous layer was further extracted with ethyl acetate and the combined organic extracts were washed with brine, dried (Na₂SO₄) and concentrated under reduced pressure to give a residue. The residue was purified by prep-HPLC to afford cis-tert-butyl 6-cyclopropyl-1,3,3a,9b-tetrahydroisochromeno[3,4-c]pyrrole-2(5H)-carboxylate (39% yield).

### 117.2 Preparation of cis-6-cyclopropyl-1,2,3,3a,5,9b-hexahydroisochromeno[3,4-c]pyrrole

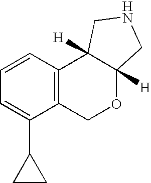

5 N HCl (0.098 ml) was added to a solution of cis-tert-butyl 6-cyclopropyl-1,3,3a,9b-tetrahydroisochromeno[3,4-c]pyrrole-2(5H)-carboxylate (9.83 μmol, 3.1 mg) in dioxane (1.0 ml)/MeOH (0.2 ml). The mixture was stirred at 100° C. for 0.5 h then concentrated under reduced pressure. The residue was passed through an SCX cartridge and then purified by basic prep-HPLC to afford cis-6-cyclopropyl-1,2,3,3a,5,9b-hexahydroisochromeno[3,4-c]pyrrole (85% yield), EI-MS: m/z=216.4 [M+H]⁺.

### EXAMPLE 118

### cis-6-Cyclopropyl-3a-methyl-1,2,3,3a,5,9b-hexahydroisochromeno[3,4-c]pyrrole 2,2,2-trifluoroacetate

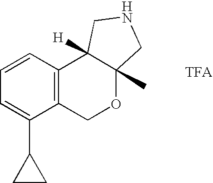

Similar procedures described in Example 117 were employed, using cis-tert-butyl 3a-methyl-6-(trifluoromethylsulfonyloxy)-1,3,3a,9b-tetrahydroisochromeno[3,4-c]pyrrole-2(5H)-carboxylate to afford cis-6-cyclopropyl-3a-methyl-1,2,3,3a,5,9b-hexahydroisochromeno[3,4-c]pyrrole 2,2,2-trifluoroacetate, EI-MS: m/z=230.4 [M+H]⁺.

### EXAMPLE 119

### cis-6-Isobutyl-1,2,3,3a,5,9b-hexahydroisochromeno[3,4-c]pyrrole

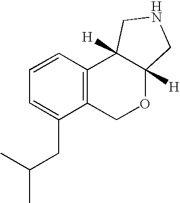

Similar procedures described in Example 111 were employed, using cis-2-benzyl-6-bromo-1,2,3,3a,5,9b-hexahydroisochromeno[3,4-c]pyrrole and isobutylboronic acid to afford cis-6-isobutyl-1,2,3,3a,5,9b-hexahydroisochromeno[3,4-c]pyrrole, EI-MS: m/z=232.4 [M+H]⁺.

### EXAMPLE 120

### cis-8-Chloro-1,2,3,3a,5,9b-hexahydroisochromeno[3,4-c]pyrrole hydrochloride

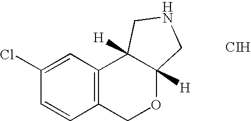

### 120.1 Preparation of 1,2-dihydrocyclobutabenzene-1-carbonitrile

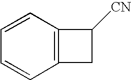

To a solution of 3-chloro-1,2-dihydrocyclobutabenzene-1-carbonitrile (15 g) and triethyl amine in EtOAc (300 ml) was added 10% Pd on carbon (1.5 g). The resulting suspension was stirred under an atmosphere of H₂ (5 bar) at 55° C. for 18 hours and then filtered through celite. The resulting filtrate was then washed with 2 N HCl (2×400 ml) and NaHCO₃ (satd, 2×200 ml) and then dried over MgSO₄ before concentrating in vacuo to afford 1,2-dihydrocyclobutabenzene-1-carbonitrile as a brown oil (13.51 g, 94%).

### 120.2 Preparation of 5-nitro-1,2-dihydrocyclobutabenzene-1-carbonitrile

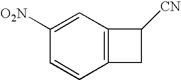

Sodium nitrate was added to conc. sulfuric acid (96%) (360 ml) with acetone/ice cooling. The resulting mixture was cooled to −5° C. and 1,2-dihydrocyclobutabenzene-1-carbonitrile was then added at a rate suffucient to keep T<10° C. After addition was complete the mixture was stirred for 30 min and then poured onto ice (1000 g) and extracted into DCM (2×) and EtOAc (1×). The combined organic extracts were then washed with sat'd NaHCO₃ (3×) and water (1×), and then dried over MgSO₄ before concentrating in vacuo to afford a crude brown solid (37 g) that was purified by silica column chromatography (eluting 10-30% EtOAc in heptane) to afford 5-nitro-1,2-dihydrocyclobutabenzene-1-carbonitrile (15.7 g, 38%) as a yellow solid.

### 120.3 Preparation of 5-amino-1,2-dihydrocyclobutabenzene-1-carbonitrile hydrochloride

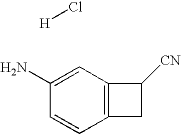

A suspension of 5-nitro-1,2-dihydrocyclobutabenzene-1-carbonitrile (8.61 mmol, 1.50 g) and 5% palladium on carbon (0.172 mmol, 0.367 g) in ethanol (80 mL) and acetic acid (0.5 mL) was stirred at rt under an atmosphere of hydrogen (balloon) for 20 hours. The spent catalyst was removed by filtration through celite. The filtrate was concentrated in vacuo and the residue was partitioned between 1 N NaOH and diethyl ether. The aqueous layer was extracted with ether and the combined organic extracts were washed with brine, dried (Na₂SO₄) and concentrated in vacuo. The residue was dissolved in diethyl ether (10 ml) and 2 M HCl in ether (5 ml) was added to the solution. The precipitate was collected by filtration and triturated in hot acetonitrile (50 ml) to afford 5-amino-1,2-dihydrocyclobutabenzene-1-carbonitrile hydrochloride (69% yield).

### 120.4 Preparation of 5-chloro-1,2-dihydrocyclobutabenzene-1-carbonitrile

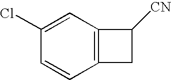

5-Amino-1,2-dihydrocyclobutabenzene-1-carbonitrile hydrochloride (10.80 mmol, 1.95 g) was dissolved in 4 M hydrochloric acid (108 mmol, 21.59 ml) and cooled in an ice-bath. A solution of sodium nitrite (17.70 mmol, 1.221 g) in water (10 ml) was added dropwise to the cooled reaction mixture. The mixture was stirred at 0° C. for 0.5 hour then added to a solution of copper(I) chloride (32.4 mmol, 3.21 g) in concentrated HCl (6 ml) at 0° C. and stirred for 10 min. The reaction mixture was diluted with water and extracted with ethyl acetate. The organic extracts were combined and washed with water then brine, dried (MgSO₄) and evaporated under vacuum to give a residue. Silica column chromatography using ethyl acetate in heptane (0 to 10%) afforded 5-chloro-1,2-dihydrocyclobutabenzene-1-carbonitrile (67% yield).

### 120.5 Preparation of 5-chloro-1,2-dihydrocyclobutabenzene-1-carboxylic acid

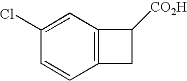

A solution of 5-chloro-1,2-dihydrocyclobutabenzene-1-carbonitrile (8.56 mmol, 1.40 g) and potassium hydroxide (42.8 mmol, 2.400 g) in ethanol (40 ml)/water (8.0 ml) was heated at reflux for 2 h. After evaporation of the organic solvent, the aqueous residue was washed with diethyl ether. The organic layer was extracted with 2 N aqueous NaOH and the combined aqueous layers were acidified with 5 N HCl and extracted with diethyl ether. The extracts were washed with brine, dried (Na₂SO₄) and concentrated in vacuo. The residue was washed with diethyl ether in heptane (0 to 20%) to afford 5-chloro-1,2-dihydrocyclobutabenzene-1-carboxylic acid (68% yield).

### 120.6 Preparation of N-benzyl-5-chloro-N-(2-hydroxyethyl)-1,2-dihydrocyclobutabenzene-1-carboxamide

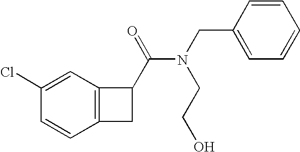

A mixture of 5-chloro-1,2-dihydrocyclobutabenzene-1-carboxylic acid (5.80 mmol, 1.06 g), 2-(benzylamino)ethanol (7.55 mmol, 1.141 g), triethylamine (11.61 mmol, 1.175 g) and cyclophos (6.97 mmol in ethyl acetate, 4.43 g) in dichloromethane was stirred at room temperature for 2 h. The reaction mixture was partitioned between dichloromethane and 2 N HCl. The aqueous layer was extracted with dichloromethane and the combined organic layers washed with water then brine, dried (Na₂SO₄) and concentrated under reduced pressure. The residue purified by silica column chromatography (eluting with ethyl acetate in heptane, 30 to 100%) to afford N-benzyl-5-chloro-N-(2-hydroxyethyl)-1,2-dihydrocyclobutabenzene-1-carboxamide (97% yield), EI-MS: m/z=316.1 [M+H]⁺.

### 120.7 Preparation of N-benzyl-5-chloro-N-(2-oxoethyl)-1,2-dihydrocyclobutabenzene-1-carboxamide

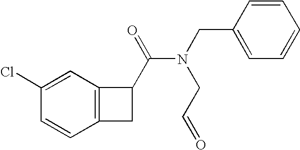

To a solution of in N-benzyl-5-chloro-N-(2-hydroxyethyl)-1,2-dihydrocyclobutabenzene-1-carboxamide (5.60 mmol, 1.77 g) in dichloromethane (8 ml) was added a solution of Dess-Martin periodinane (5.89 mmol in dichloromethane, 16.64 g). The mixture was stirred for 2 h at room temperature. Saturated aqueous NaHCO₃ (30 ml) was added and the mixture was stirred for 30 minutes, diluted with additional dichloromethane (50 ml) and the phases separated. The aqueous layer was extracted with dichloromethane and the combined organic layers were washed with water and then brine, dried (Na₂SO₄) and concentrated under reduced pressure. The residue was purified by silica column chromatography (eluting with ethyl acetate in heptane, 20 to 50%) to afford N-benzyl-5-chloro-N-(2-oxoethyl)-1,2-dihydrocyclobutabenzene-1-carboxamide (81% yield), EI-MS: m/z=314.1 [M+H]⁺.

### 120.8 Preparation of cis-2-benzyl-8-chloro-3,3a,5,9b-tetrahydroisochromeno[3,4-c]pyrrol-1(2H)-one and trans-2-benzyl-8-chloro-3,3a,5,9b-tetrahydroisochromeno[3,4-c]pyrrol-1(2H)-one

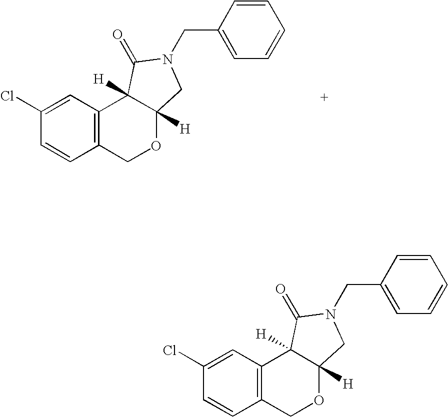

A solution of N-benzyl-5-chloro-N-(2-oxoethyl)-1,2-dihydrocyclobutabenzene-1-carboxamide (4.53 mmol, 1.42 g) in bromobenzene (15 ml) was subjected to microwave irradiation at 210° C. for 30 minutes. The solvent was removed under reduced pressure and the residue purified by silica column chromatography (eluting with ethyl acetate in heptane, 25 to 50%) to afford cis-2-benzyl-8-chloro-3,3a,5,9b-tetrahydroisochromeno[3,4-c]pyrrol-1(2H)-one (64% yield), followed by trans-2-benzyl-8-chloro-3,3a,5,9b-tetrahydroisochromeno[3,4-c]pyrrol-1(2H)-one (5% yield).

### 120.8 Preparation of cis-2-benzyl-8-chloro-1,2,3,3a,5,9b-hexahydroisochromeno[3,4-c]pyrrole

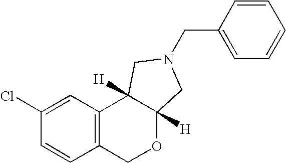

1 M Borane in THF (8.61 mmol, 8.61 ml) was added dropwise to a solution of cis-2-benzyl-8-chloro-3,3a,5,9b-tetrahydroisochromeno[3,4-c]pyrrol-1(2H)-one (2.87 mmol, 901 mg) in THF (15 ml) and the mixture was stirred at room temperature for 1 h and then at reflux for 4 h. Further 1 M borane in THF (8.61 mmol, 8.61 ml) was added and the mixture was stirred at reflux for a further 5 h. The mixture was neutralised with 4 N aqueous NaOH then extracted with diethyl ether, washed with brine, dried (Na₂SO₄) and concentrated under reduced pressure to afford a residue. The crude residue was purified by silica column chromatography (eluting with ethyl acetate in heptane, 0 to 50%) to afford cis-2-benzyl-8-chloro-1,2,3,3a,5,9b-hexahydroisochromeno[3,4-c]pyrrole (45% yield), EI-MS: m/z=300.1 [M+H]⁺.

### 120.9 Preparation of cis-8-chloro-1,2,3,3a,5,9b-hexahydroisochromeno[3,4-c]pyrrole hydrochloride

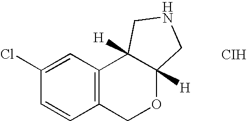

A solution of cis-2-benzyl-8-chloro-1,2,3,3a,5,9b-hexahydroisochromeno[3,4-c]pyrrole (1.308 mmol, 392 mg) and 1-chloroethyl chloroformate (6.54 mmol, 0.705 ml, 935 mg) in toluene (8 ml) was subjected to microwave irradiation at 160° C. for 30 minutes, then methanol was added and the mixture irradiated at 160° C. for 5 minutes. After addition of excess saturated aqueous NaHCO₃ (40 ml), the mixture was extracted with ethyl acetate (20 ml, ×3), washed with brine, dried (Na₂SO₄) and concentrated under reduced pressure to give a residue. The crude residue was passed through an SCX cartridge to affrod cis-8-chloro-1,2,3,3a,5,9b-hexahydroisochromeno[3,4-c]pyrrole (44% yield). A portion was purified by basic prep-HPLC and the product converted to the HCl salt (using 5N HCl) to afford cis-8-chloro-1,2,3,3a,5,9b-hexahydroisochromeno[3,4-c]pyrrole hydrochloride, EI-MS: m/z=210.3 [M+H]⁺.

### EXAMPLE 121

### cis-7-Bromo-8-chloro-1,2,3,3a,5,9b-hexahydroisochromeno[3,4-c]pyrrole hydrochloride and cis-9-bromo-8-chloro-1,2,3,3a,5,9b-hexahydroisochromeno[3,4-c]pyrrole hydrochloride

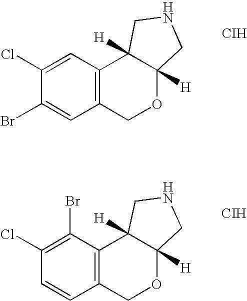

N-Bromosuccinimide (0.191 mmol, 34.0 mg) was added to a solution of cis-8-chloro-1,2,3,3a,5,9b-hexahydroisochromeno[3,4-c]pyrrole (0.191 mmol, 40 mg) in sulfuric acid (1 ml). The mixture was stirred at room temperature for 5 h in the dark. The reaction mixture was poured into ice-water (15 ml) and diethyl ether added. The aqueous layer was basified with 4 N NaOH and extracted with diethyl ether. The combined organic extracts were washed with brine, dried (Na₂SO₄) and concentrated under reduced pressure to afford a residue. The residue was passed through an SCX cartridge then purified by basic prep-HPLC to afford the desired products, which were converted to the corresponding HCl salts (using HCl in ether) to afford cis-7-bromo-8-chloro-1,2,3,3a,5,9b-hexahydroisochromeno[3,4-c]pyrrole hydrochloride (14% yield), EI-MS: m/z=290.0 [M+H]⁺, followed by cis-9-bromo-8-chloro-1,2,3,3a,5,9b-hexahydroisochromeno[3,4-c]pyrrole hydrochloride (4% yield), EI-MS: m/z=290.0 [M+H]⁺.

### EXAMPLE 122

### cis-8-Chloro-7-methyl-1,2,3,3a,5,9b-hexahydroisochromeno[3,4-c]pyrrole hydrochloride

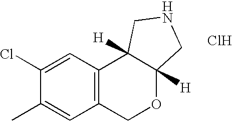

A mixture of cis-7-bromo-8-chloro-1,2,3,3a,5,9b-hexahydroisochromeno[3,4-c]pyrrole hydrochloride (0.023 mmol, 7.5 mg), trimethylboroxine (0.046 mmol, 6.45 μL, 5.79 mg), tetrakis(triphenylphosphine)palladium(0) (2.307 μmol, 2.67 mg) and potassium carbonate (0.046 mmol, 6.38 mg) in dioxane (1 mL) was subjected to microwave irradiation at 120° C. for 15 minutes. The reaction mixture was passed through an SCX cartridge and then purified by basic prep-HPLC. The product was converted to the HCl salt to afford cis-8-chloro-7-methyl-1,2,3,3a,5,9b-hexahydroisochromeno[3,4-c]pyrrole hydrochloride (45% yield), EI-MS: m/z=224.1 [M+H]⁺.

### EXAMPLE 123

### cis-7-Chloro-8-methyl-2,3,4,4a,6,10b-hexahydro-1H-isochromeno[4,3-c]pyridine

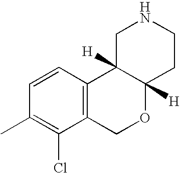

A mixture of cis-8-bromo-7-chloro-2,3,4,4a,6,10b-hexahydro-1H-isochromeno[4,3-c]pyridine (0.050 mmol, 15 mg), tetrakis(triphenylphosphine)palladium(0) (4.96 μmol, 5.79 mg), trimethylboroxine (0.055 mmol, 7.70 μl, 6.91 mg) and potassium carbonate (0.149 mmol, 20.76 mg) in 1,4-dioxane (1 ml) was heated in a microwave at 130° C. for 20 min. Water was then added and the resulting mixture was extracted with DCM, then passed through an SCX column (1 g) to give a white solid that was purified by prep-HPLC (basic) to give cis-7-chloro-8-methyl-2,3,4,4a,6,10b-hexahydro-1H-isochromeno[4,3-c]pyridine (2.6 mg, 22%) as a white solid EI-MS: m/z=238.1, 240.4 [M+H]⁺.

### EXAMPLE 124

### trans-7-Chloro-8-methyl-2,3,4,4a,6,10b-hexahydro-1H-isochromeno[4,3-c]pyridine

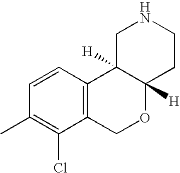

### 124.1 Preparation of trans-2-benzyl-7-chloro-8-methyl-2,3,4,4a,6,10b-hexahydro-1H-isochromeno[4,3-c]pyridine

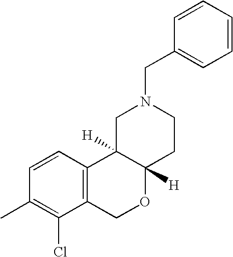

A mixture of trans-2-benzyl-8-bromo-7-chloro-2,3,4,4a,6,10b-hexahydro-1H-isochromeno[4,3-c]pyridine (0.166 mmol, 65 mg), tetrakis(triphenylphosphine)palladium(0) (0.017 mmol, 19.32 mg), trimethylboroxine (0.184 mmol, 26 μl, 23.35 mg) and potassium carbonate (0.497 mmol, 69.3 mg) in 1,4-dioxane (1 ml) was heated in a microwave at 130° C. for 20 min. Water was then added and the resulting mixture was extracted with DCM, then passed through a SCX column (1 g) to give a white solid that was purified by silica column chromatography (4 g silica, eluting with EtOAc in heptane, 0-20%) to give trans-2-benzyl-7-chloro-8-methyl-2,3,4,4a,6,10b-hexahydro-1H-isochromeno[4,3-c]pyridine as a yellow solid (50 mg, 92%), EI-MS: m/z=328.3, 330.0 [M+H]⁺.

### 124.2 Preparation of trans-7-chloro-8-methyl-2,3,4,4a,6,10b-hexahydro-1H-isochromeno[4,3-c]pyridine

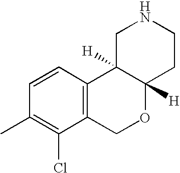

A solution of trans-2-benzyl-7-chloro-8-methyl-2,3,4,4a,6,10b-hexahydro-1H-isochromeno[4,3-c]pyridine (0.107 mmol, 35 mg) and 1-chloroethyl chloroformate (0.533 mmol, 59 μl, 78 mg) in toluene (1 ml) was heated in a microwave reactor at 160° C. for 0.5 h. MeOH (0.5 ml) was added and the reaction was heated further in a microwave reactor at 160° C. for 5 min. The resulting mixture was passed through an SCX column (1 g) to give a white solid that was purified by prep-LCMS (basic) to give trans-7-chloro-8-methyl-2,3,4,4a,6,10b-hexahydro-1H-isochromeno[4,3-c]pyridine (17.5 mg, 69%) as a white solid, EI-MS: m/z=238.1, 240.0 [M+H]⁺.

### EXAMPLE 125

### cis-7-Chloro-10-methyl-2,3,4,4a,6,10b-hexahydro-1H-isochromeno[4,3-c]pyridine

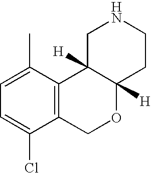

Similar procedures to that in Example 122 above were employed, using cis-10-bromo-7-chloro-2,3,4,4a,6,10b-hexahydro-1H-isochromeno[4,3-c]pyridine to afford cis-7-chloro-10-methyl-2,3,4,4a,6,10b-hexahydro-1H-isochromeno[4,3-c]pyridine, EI-MS: m/z=238.1, 240.4 [M+H]⁺.

### EXAMPLE 126

### trans-7-Chloro-10-methyl-2,3,4,4a,6,10b-hexahydro-1H-isochromeno[4,3-c]pyridine

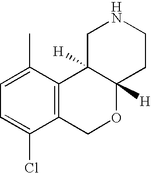

Similar procedures to that in Example 122 above were employed, using trans-10-bromo-7-chloro-2,3,4,4a,6,10b-hexahydro-1H-isochromeno[4,3-c]pyridine to afford trans-7-chloro-10-methyl-2,3,4,4a,6,10b-hexahydro-1H-isochromeno[4,3-c]pyridine, EI-MS: m/z=238.3, 240.0 [M+H]⁺.

### EXAMPLE 127

### cis-8,9-Dichloro-1,2,3,3a,5,9b-hexahydroisochromeno[3,4-c]pyrrole hydrochloride

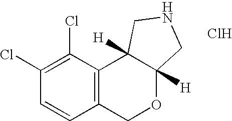

N-Chlorosuccinimide (0.279 mmol, 37.2 mg) was added to a solution of cis-8-chloro-1,2,3,3a,5,9b-hexahydroisochromeno[3,4-c]pyrrole (0.279 mmol, 58.4 mg) in sulfuric acid (1 ml). The mixture was stirred at room temperature for 3 h in the dark. The reaction mixture was then poured into ice-water (15 ml) and diethyl ether added. The aqueous layer was basified with 4 N NaOH and extracted with diethyl ether. The combined organic layers were washed with brine, dried (Na₂SO₄) and concentrated under reduced pressure to afford a residue. The residue was passed through an SCX cartridge and then purified by basic prep-HPLC to afford the desired product, which was converted to the corresponding HCl salt to afford cis-8,9-dichloro-1,2,3,3a,5,9b-hexahydroisochromeno[3,4-c]pyrrole hydrochloride (7% yield), EI-MS: m/z=244.4 [M+H]⁺.

### EXAMPLE 128

### trans-6-(Benzyloxy)-3a-methyl-1,2,3,3a,5,9b-hexahydroisochromeno[3,4-c]pyrrole 2,2,2-trifluoroacetate

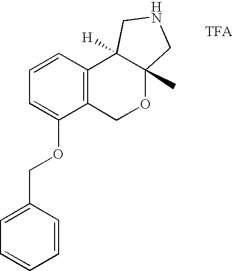

### 128.1 Preparation of 2-bromo-6-hydroxybenzaldehyde

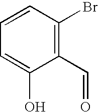

Sodium hydroxide (462 g, 11.6 mol) was dissolved in the water (650 ml) and 3-bromophenol (250 g, 1.4 mol) was added. The resulting suspension was stirred and heated to 75° C. during which time a solution formed. Chloroform (231 ml, 2.89 mol) was then added dropwise over 45 minutes and the mixture was heated at 75° C. until signs of chloroform reflux had gone (˜35 minutes). The orange-brown suspension was then cooled to <5° C. and 2 N HCl (1.5 L) was added dropwise keeping temperature <15° C. The mixture was then adjusted to ˜pH 3 with 5 N HCl and then extracted into ethyl acetate (4×500 ml). The combined organic extracts were dried over magnesium sulphate, filtered and evaporated under reduced pressure to give a dark oil. The crude product was stirred with dichloromethane (500 ml) and the insoluble material was removed by filtration. The filtrate was then passed through a silica column (˜1.6 kg) (eluting with ethyl acetate in heptane, 1-10%) to give 2-bromo-6-hydroxybenzaldehyde (100 g, 35%).

### 128.2 Preparation of 2-(benzyloxy)-6-bromobenzaldehyde

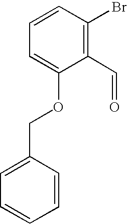

Benzyl bromide (91 g, 531 mmol) and potassium hydroxide pellets (29.8 g, 531 mmol) were stirred in THF and 2-bromo-6-hydroxybenzaldehyde (97 g, 483 mmol) was added in one portion. The mixture was heated to reflux, during which time a yellow suspension was formed, and stirred at reflux overnight. The resulting suspension was filtered, the insoluble material was washed with THF, and the filtrate was evaporated to dryness. The residue was dissolved in ether (2 L), washed with 2 N potassium hydroxide (2×1 L), dried over magnesium sulphate and evaporated to dryness. The residue was dissolved in toluene (75 ml), stirred, and heptane (1 L) slowly added giving a suspension which was placed in fridge over weekend. The resulting solid was collected by filtration, washed with heptane and dried in a vacuum oven at 35° C. to give 2-(benzyloxy)-6-bromobenzaldehyde (98.45 g, 70%) as a white solid.

### 128.3 Preparation of 3-(2-(benzyloxy)-6-bromophenyl)-2-cyanoacrylic acid

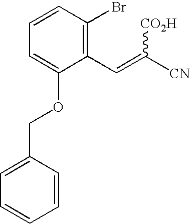

2-(Benzyloxy)-6-bromobenzaldehyde (59.6 g, 205 mmol), cyanoacetic acid (17.41 g, 205 mmol), ammonium acetate (3.16, 40.9 mmol) and 4 Å molecular sieves (60 g) were stirred together in toluene (320 ml) and dry pyridine (28.8 ml) under nitrogen. The reaction mixture was stirred at reflux under Dean & Stark conditions for 50 minutes and then cooled to room temperature. The reaction mixture was filtered through dicalite and the resulting filtrate was evaporated to dryness to give a viscous yellow oil. The crude product was dissolved in ethyl acetate (˜250 ml) and extracted with 0.5 N sodium hydroxide (1×200 ml, 2×100 ml). The combined basic extracts were acidified with 5 N hydrochloric acid and extacted with ethyl acetate (4×100 ml). These organic extracts were combined and washed with water (3×75 ml) then dried over sodium sulphate, filtered and evaporated to give 3-(2-(benzyloxy)-6-bromophenyl)-2-cyanoacrylic acid (61.7 g, 84%).

### 128.4 Preparation of 3-(2-(benzyloxy)-6-bromophenyl)-2-cyanopropanoic acid

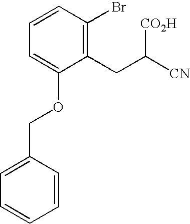

3-(2-(Benzyloxy)-6-bromophenyl)-2-cyanoacrylic acid (78 g, 218 mmol), methanol (930 ml), and saturated aqueous sodium bicarbonate (211 ml) were cooled to below 15° C. and sodium borohydride was added portionwise over ˜2 hours maintaining temperature at 15° C. After the addition was complete the cooling bath was removed and the mixture was stirred then at room temperature for 30 minutes. The reaction mixture was then evaporated to a residue that was dissolved in water (150 ml), acidified with 5 N HCl (˜60 ml) and extracted with ether (3×100 ml). The combined organic extracts were washed with water (3×50 ml), dried over sodium sulphate, filtered and concentrated in vacuo to give 3-(2-(benzyloxy)-6-bromophenyl)-2-cyanopropanoic acid (77.37 g, 99%) as a yellow oil that solidified upon standing to give an off-white solid.

### 128.5 Preparation of 3-(2-(benzyloxy)-6-bromophenyl)propanenitrile

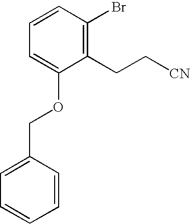

A solution of 3-(2-(benzyloxy)-6-bromophenyl)-2-cyanopropanoic acid (77.2 g, 214 mmol) in DMA (88.7 ml) was heated to 140-150° C. for ˜1.5 hours. The reaction mixture was cooled to room temperature and water (890 ml) was added. The resulting suspension was transferred to a separating funnel and attempts made to extract the solid into ether (4×250 ml). A final extract of ether containing some methylene chloride eventually dissolved the remaining solid. The organic extracts were combined and washed with saturated sodium bicarbonate (75 ml) followed by water (3×75 ml). The organic extract was dried over sodium sulphate, filtered and evaporated to dryness to give an off-white solid that was purified by silica column chromatography (eluting with DCM in heptane, 50%) to afford 3-(2-(benzyloxy)-6-bromophenyl)propanenitrile (58.3 g, 86%).

### 128.6 Preparation of 3-(benzyloxy)-1,2-dihydrocyclobutabenzene-1-carbonitrile

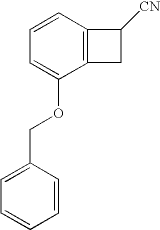

A dried 1 L 3-neck flask was fitted with a magnetic stirrer, drikold condenser and thermometer, was cooled to −78° C. under N₂. Ammonia was condensed from a cylinder into a separate precooled dried flask until enough was collected to give the required amount. The ammonia was then allowed to distil over from the first flask into the reaction flask via a connecting tube until ˜340 ml was collected.

The ammonia was stirred at −76° C. and sodium amide (4.84, 124 mmol) was added in one portion. The mixture was stirred for 10 minutes and then 3-(2-(benzyloxy)-6-bromophenyl)propanenitrile (10 g, 31.6 mmol) was added portionwise over ˜5 minutes. The resulting yellowish suspension was allowed to warm up until reflux was established and stirred for 6 hours before being neutralised with solid ammonium nitrate (10.96 g, 137 mmol). The ammonia was removed under a stream of N₂ and water (200 ml) was added. The resulting mixture was extracted with methylene chloride (3×100 ml) and the organic extracts were combined and washed with 1 N HCl (75 ml), water (3×75 ml) and brine (75 ml). The organic extract was then dried over sodium sulphate, filtered and concentrated in vacuo to give 3-(benzyloxy)-1,2-dihydrocyclobutabenzene-1-carbonitrile (7.48 g, quant) as a brown oil which solidified upon standing.

### 128.7 Preparation of 3-(benzyloxy)-1,2-dihydrocyclobutabenzene-1-carboxylic acid

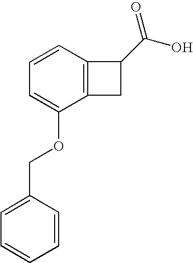

3-(Benzyloxy)-1,2-dihydrocyclobutabenzene-1-carbonitrile (11.15 g, 47.4 mmol) was dissolved in a solution of ethanol (70 ml) and potassium hydroxide (13.3 g, 237 mmol) dissolved in water (14 ml) was added. The mixture was heated at reflux for 2 h. After evaporation of the solvent, the residue was partitioned between water and ether. The organic layer was extracted with 2 N NaOH (2×75 ml) and the combined aqueous extracts were acidified with 5 N HCl then extracted with ether (3×100 ml). The combined ether extracts were washed with brine, dried over sodium sulphate and evaporated to dryness to give a brown solid. The solid was triturated with heptane:ether 5:1 then collected by filtration and dried in vacuum oven at 45° C. to give 3-(benzyloxy)-1,2-dihydrocyclobutabenzene-1-carboxylic acid (10.82 g).

### 128.8 Preparation of trans-2-benzyl-6-(benzyloxy)-3a-methyl-3,3a,5,9b-tetrahydroisochromeno[3,4-c]pyrrol-1(2H)-one and cis-2-benzyl-6-(benzyloxy)-3a-methyl-3,3a,5,9b-tetrahydroisochromeno[3,4-c]pyrrol-1(2H)-one

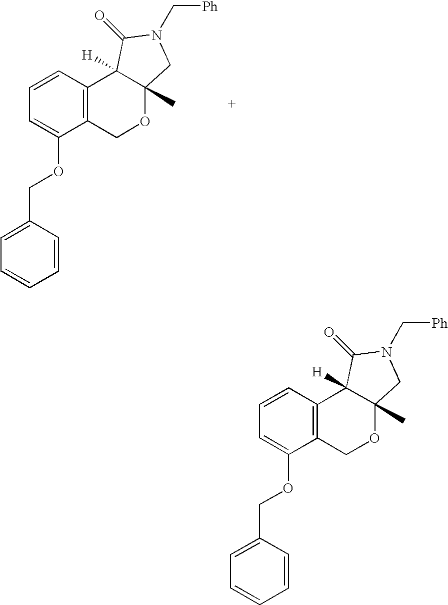

Similar protocols to procedures in Example 40 were employed, using 3-(benzyloxy)-1,2-dihydrocyclobutabenzene-1-carboxylic acid to afford cis-2-benzyl-6-(benzyloxy)-3a-methyl-3,3a,5,9b-tetrahydroisochromeno[3,4-c]pyrrol-1(2H)-one, EI-MS: m/z=400.3 [M+H]⁺ and trans-2-benzyl-6-(benzyloxy)-3a-methyl-3,3a,5,9b-tetrahydroisochromeno[3,4-c]pyrrol-1(2H)-one, EI-MS: m/z=400.3 [M+H]⁺

### 128.9 trans-2-benzyl-6-(benzyloxy)-3a-methyl-1,2,3,3a,5,9b-hexahydroisochromeno[3,4-c]pyrrole

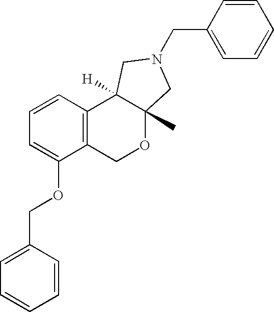

To a solution of trans-2-benzyl-6-(benzyloxy)-3a-methyl-3,3a,5,9b-tetrahydroisochromeno[3,4-c]pyrrol-1(2H)-one (1.277 mmol, 510 mg) in THF (5 ml) was added 1 M borane in THF (3.84 mmol, 3.84 ml) dropwise. The reaction mixture was stirred at 0.5 h and then at reflux for a further 3 h. 5 N HCl (5 ml) was then added at rt, and the reaction mixture was again stirred at reflux for a further 4 h and then cooled to rt and neutralised with 4 N aq. NaOH. The resulting mixture was then extracted with Et₂O (3×). The combined organic extracts were washed with brine, dried over Na₂SO₄ and concentrated in vacuo to give a crude oil that was passed through an SCX cartridge to afford trans-2-benzyl-6-(benzyloxy)-3a-methyl-1,2,3,3a,5,9b-hexahydroisochromeno[3,4-c]pyrrole (70 mg, 15%), EI-MS: m/z=386.0 [M+H]⁺.

### 128.10 Preparation of trans-methyl 6-(benzyloxy)-3a-methyl-1,3,3a,9b-tetrahydroisochromeno[3,4-c]pyrrole-2(5H)-carboxylate

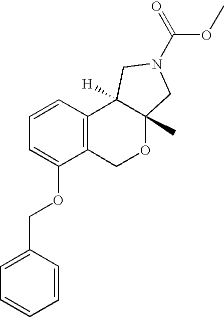

A solution of trans-2-benzyl-6-(benzyloxy)-3a-methyl-1,2,3,3a,5,9b-hexahydroisochromeno[3,4-c]pyrrole (2.57 mmol, 990 mg) and 1-chloroethyl chloroformate (10.27 mmol, 1.108 ml, 1469 mg) in toluene (20 ml) was subjected to microwave irradiation at 160° C. for 30 minutes. MeOH (1 ml) was added and the reaction mixture was subjected to microwave irradiation at 160° C. for 5 minutes. Saturated aqueous NaHCO₃ (40 ml) was added and the mixture was extracted with EtOAc (3×20 ml). The combined organic extracts were washed with brine, dried (Na₂SO₄) and concentrated under reduced pressure to give a residue that was passed through an SCX column to afford trans-methyl 6-(benzyloxy)-3a-methyl-1,3,3a,9b-tetrahydroisochromeno[3,4-c]pyrrole-2(5H)-carboxylate (374 mg, 41%), EI-MS: m/z=268.1 [M+H]⁺.

### 128.11 Preparation of trans-6-(benzyloxy)-3a-methyl-1,2,3,3a,5,9b-hexahydroisochromeno[3,4-c]pyrrole 2,2,2-trifluoroacetate

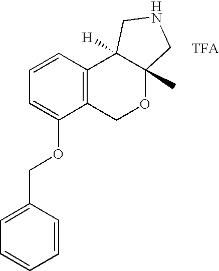

To a solution of trans-methyl 6-(benzyloxy)-3a-methyl-1,3,3a,9b-tetrahydroisochromeno[3,4-c]pyrrole-2(5H)-carboxylate (1.047 mmol, 370 mg) in MeOH was added potassium hydroxide (10.47 mmol, 1.047 ml) in water and the mixture was subjected to microwave irradiation at 150° C. for 30 minutes. The resulting mixture was extracted with DCM (20 ml, ×3) and the combined organic extracts were washed with brine, dried (Na₂SO₄) and concentrated under reduced pressure and then passed through an SCX column before purifying by prep-HPLC (acidic modifier) to afford trans-6-(benzyloxy)-3a-methyl-1,2,3,3a,5,9b-hexahydroisochromeno[3,4-c]pyrrole 2,2,2-trifluoroacetate (6.6 mg, 2%), EI-MS: m/z=295.5 [M+H]⁺.

### EXAMPLE 129

### cis-6-Bromo-3a-methyl-1,2,3,3a,5,9b-hexahydroisochromeno[3,4-c]pyrrole 2,2,2-trifluoroacetate

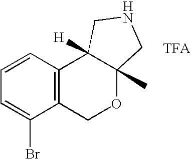

A mixture of cis-tert-butyl 3a-methyl-6-(trifluoromethylsulfonyloxy)-1,3,3a,9b-tetrahydroisochromeno[3,4-c]pyrrole-2(5H)-carboxylate (0.046 mmol, 20 mg), and nickel(II) bromide (0.229 mmol, 50.0 mg) in N-methyl-2-pyrrolidinone (3 ml) was subjected to microwave irradiation at 210° C. for 20 min. The reaction mixture was partitioned between ethyl acetate and water, and the aqueous layer was further extracted with ethyl acetate. The combined organic layers were washed with brine, dried (Na₂SO₄) and concentrated under reduced pressure to give a residue that was passed through an SCX cartridge and then purified by acidic prep-HPLC afforded cis-6-bromo-3a-methyl-1,2,3,3a,5,9b-hexahydroisochromeno[3,4-c]pyrrole 2,2,2-trifluoroacetate (28% yield), EI-MS: m/z=268.1 [M+H]⁺.

### EXAMPLE 130

### cis-6-Methoxy-3a-methyl-1,2,3,3a,5,9b-hexahydroisochromeno[3,4-c]pyrrole hydrochloride

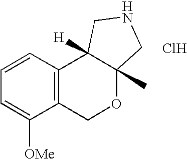

### 130.1 Preparation of cis-tert-butyl 6-methoxy-3a-methyl-1,3,3a,9b-tetrahydroisochromeno[3,4-c]pyrrole-2(5H)-carboxylate

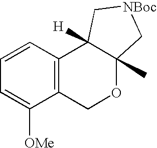

Trimethylsilyldiazomethane (0.196 mmol, 0.098 ml) was added dropwise to a solution of cis-tert-butyl 6-hydroxy-3a-methyl-1,3,3a,9b-tetrahydroisochromeno[3,4-c]pyrrole-2(5H)-carboxylate (0.164 mmol, 50 mg), DIPEA (0.246 mmol, 0.041 ml, 32.1 mg) in MeOH (0.5 ml) and acetonitrile (4.5 ml). The mixture was stirred at room temperature for 4 h then additional DIPEA (0.246 mmol, 0.041 ml, 32.1 mg) and trimethylsilyldiazomethane (0.196 mmol, 0.098 ml) was added and mixture was stirred for a further 16 h. The reaction mixture was concentrated in vacuo and purified by prep-HPLC to afford cis-tert-butyl 6-methoxy-3a-methyl-1,3,3a,9b-tetrahydroisochromeno[3,4-c]pyrrole-2(5H)-carboxylate (37% yield).

### 130.2 Preparation of cis-6-methoxy-3a-methyl-1,2,3,3a,5,9b-hexahydroisochromeno[3,4-c]pyrrole hydrochloride

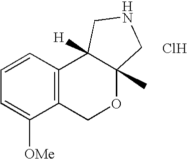

5 N HCl (0.120 ml) was added to a solution of cis-tert-butyl 6-methoxy-3a-methyl-1,3,3a,9b-tetrahydroisochromeno[3,4-c]pyrrole-2(5H)-carboxylate (0.060 mmol, 19.2 mg) in dioxane/MeOH. The mixture was stirred at 70° C. for 1 h then the solvent was removed under reduced pressure to afford cis-6-methoxy-3a-methyl-1,2,3,3a,5,9b-hexahydroisochromeno[3,4-c]pyrrole hydrochloride (72% yield), EI-MS: m/z=220.6 [M+H]⁺.

### EXAMPLE 131

### trans-6-Methoxy-3a-methyl-1,2,3,3a,5,9b-hexahydroisochromeno[3,4-c]pyrrole hydrochloride

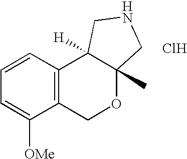

Similar procedures described in Example 130 were employed, using trans-tert-butyl 6-hydroxy-3a-methyl-1,3,3a,9b-tetrahydroisochromeno[3,4-c]pyrrole-2(5H)-carboxylate to afford trans-6-methoxy-3a-methyl-1,2,3,3a,5,9b-hexahydroisochromeno[3,4-c]pyrrole hydrochloride, EI-MS: m/z=220.4 [M+H]⁺.

### EXAMPLE 132

### cis-6-Methoxy-1,2,3,3a,5,9b-hexahydroisochromeno[3,4-c]pyrrole hydrochloride

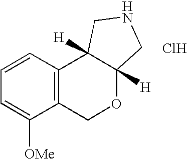

### 132.1 Preparation of cis-tert-butyl 6-methoxy-1,3,3a,9b-tetrahydroisochromeno[3,4-c]pyrrole-2(5H)-carboxylate

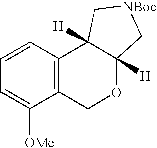

To a solution of cis-tert-butyl 6-hydroxy-1,3,3a,9b-tetrahydroisochromeno[3,4-c]pyrrole-2(5H)-carboxylate (0.051 mmol, 15 mg) in dichloromethane (3 ml) was added potassium carbonate (0.257 mmol, 35.6 mg) followed by iodomethane (0.103 mmol, 6.41 μl, 14.62 mg). The mixture was subjected to microwave irradiation at 60° C. for 15 minutes then triethylamine (10.4 mg, 0.103 mmol) was added and the mixture was further irradiated at 60° C. for 15 minutes. The mixture was then partitioned between water and dichloromethane. The organic phase was washed with brine, dried (Na₂SO₄) and concentrated under reduced pressure. The residue was purified by silica column chromatography (eluting with ethyl acetate in heptane, 25 to 40%) to afford cis-tert-butyl 6-methoxy-1,3,3a,9b-tetrahydroisochromeno[3,4-c]pyrrole-2(5H)-carboxylate (23% yield).

### 132.2 Preparation of cis-6-methoxy-1,2,3,3a,5,9b-hexahydroisochromeno[3,4-c]pyrrole hydrochloride

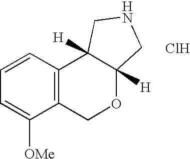

5 N HCl was added to a solution of cis-tert-butyl 6-methoxy-1,3,3a,9b-tetrahydroisochromeno[3,4-c]pyrrole-2(5H)-carboxylate (0.011 mmol, 3.5 mg) in dioxane/MeOH. The mixture was stirred at 100° C. for 0.5 h concentrated under reduced pressure. The residue was passed through an SCX cartridge and purified by basic prep-HPLC to give the desired product which was converted to the HCl salt with 2 M HCl in diethyl ether to afford cis-6-methoxy-1,2,3,3a,5,9b-hexahydroisochromeno[3,4-c]pyrrole hydrochloride (90% yield), EI-MS: m/z=206.4 [M+H]⁺.

### EXAMPLE 133

### cis-6-Ethoxy-3a-methyl-1,2,3,3a,5,9b-hexahydroisochromeno[3,4-c]pyrrole hydrochloride

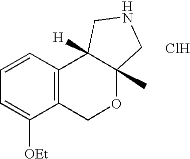

### 133.1 Preparation of cis-tert-butyl 6-ethoxy-3a-methyl-1,3,3a,9b-tetrahydroisochromeno[3,4-c]pyrrole-2(5H)-carboxylate

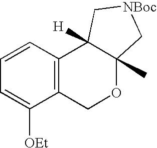

Sodium hydride (0.262 mmol, 10.48 mg) was added to a solution of cis-tert-butyl 6-hydroxy-3a-methyl-1,3,3a,9b-tetrahydroisochromeno[3,4-c]pyrrole-2(5H)-carboxylate (0.131 mmol, 40 mg) in DMF (3 ml). The mixture was stirred at room temperature for 10 min then iodoethane (0.262 mmol, 40.9 mg) was added. The mixture was stirred at room temperature for a further 1 h. The mixture was partitioned between water and dichloromethane. The organic phase was washed with brine, dried (Na₂SO₄) and concentrated under reduced pressure to afford cis-tert-butyl 6-ethoxy-3a-methyl-1,3,3a,9b-tetrahydroisochromeno[3,4-c]pyrrole-2(5H)-carboxylate (76% yield).

### 133.2 Preparation of cis-6-ethoxy-3a-methyl-1,2,3,3a,5,9b-hexahydroisochromeno[3,4-c]pyrrole hydrochloride

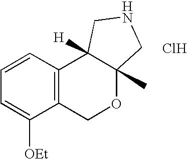

5 N HCl (0.198 ml) was added to a solution of cis-tert-butyl 6-ethoxy-3a-methyl-1,3,3a,9b-tetrahydroisochromeno[3,4-c]pyrrole-2(5H)-carboxylate (0.099 mmol, 33 mg) in dioxane/MeOH. The mixture was stirred at 70° C. for 1 h then concentrated under reduced pressure to afford cis-6-ethoxy-3a-methyl-1,2,3,3a,5,9b-hexahydroisochromeno[3,4-c]pyrrole hydrochloride (97% yield), EI-MS: m/z=234.4 [M+H]⁺.

### EXAMPLE 134

### cis-6-Ethoxy-1,2,3,3a,5,9b-hexahydroisochromeno[3,4-c]pyrrole hydrochloride

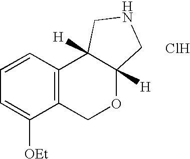

Similar procedures as described in Example 133 were employed, using cis-tert-butyl 6-hydroxy-1,3,3a,9b-tetrahydroisochromeno[3,4-c]pyrrole-2(5H)-carboxylate and iodoethane to afford cis-6-ethoxy-1,2,3,3a,5,9b-hexahydroisochromeno[3,4-c]pyrrole hydrochloride, EI-MS: m/z=220.6 [M+H]⁺.

### EXAMPLE 135

### trans-6-Isopropoxy-3a-methyl-1,2,3,3a,5,9b-hexahydroisochromeno[3,4-c]pyrrole hydrochloride

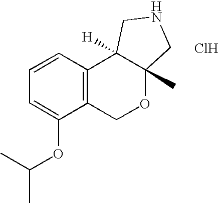

Similar procedures described in Example 133 were employed, using trans-tert-butyl 6-hydroxy-3a-methyl-1,3,3a,9b-tetrahydroisochromeno[3,4-c]pyrrole-2(5H)-carboxylate and 2-bromopropane to afford trans-6-isopropoxy-3a-methyl-1,2,3,3a,5,9b-hexahydroisochromeno[3,4-c]pyrrole hydrochloride, EI-MS: m/z=248.6 [M+H]⁺.

### EXAMPLE 136

### cis-6-Isopropoxy-1,2,3,3a,5,9b-hexahydroisochromeno[3,4-c]pyrrole hydrochloride

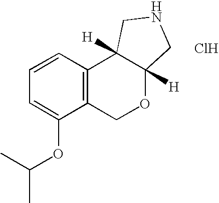

Similar procedures as described in Example 133 were employed, using cis-tert-butyl 6-hydroxy-1,3,3a,9b-tetrahydroisochromeno[3,4-c]pyrrole-2(5H)-carboxylate and 2-iodopropane to afford cis-6-isopropoxy-1,2,3,3a,5,9b-hexahydroisochromeno[3,4-c]pyrrole hydrochloride, EI-MS: m/z=234.1 [M+H]⁺.

### EXAMPLE 137

### trans-3a-Methyl-6-(prop-1-en-2-yl)-1,2,3,3a,5,9b-hexahydroisochromeno[3,4-c]pyrrole 2,2,2-trifluoroacetate

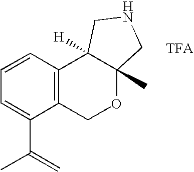

5 N HCl (0.041 ml) was added to a solution of trans-tert-butyl 3a-methyl-6-(prop-1-en-2-yl)-1,3,3a,9b-tetrahydroisochromeno[3,4-c]pyrrole-2(5H)-carboxylate (0.021 mmol, 6.8 mg) in dioxane/MeOH. The mixture was stirred at 70° C. for 1 h then concentrated under reduced pressure. The residue was purified by acidic prep-HPLC to afford trans-3a-methyl-6-(prop-1-en-2-yl)-1,2,3,3a,5,9b-hexahydroisochromeno[3,4-c]pyrrole 2,2,2-trifluoroacetate (49% yield), EI-MS: m/z=230.4 [M+H]⁺.

### EXAMPLE 138

### cis-3a-Methyl-6-(2-methylprop-1-enyl)-1,2,3,3a,5,9b-hexahydroisochromeno[3,4-c]pyrrole hydrochloride

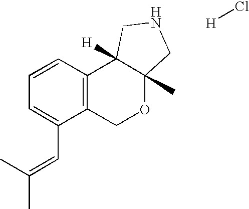

Similar procedures described in Example 137 were employed, using cis-tert-butyl 3a-methyl-6-(2-methylprop-1-enyl)-1,3,3a,9b-tetrahydroisochromeno[3,4-c]pyrrole-2(5H)-carboxylate to afford cis-3a-methyl-6-(2-methylprop-1-enyl)-1,2,3,3a,5,9b-hexahydroisochromeno[3,4-c]pyrrole hydrochloride, EI-MS: m/z=244.4 [M+H]⁺.

### EXAMPLE 139

### cis-3a-Methyl-6-((Z)-prop-1-enyl)-1,2,3,3a,5,9b-hexahydroisochromeno[3,4-c]pyrrole hydrochloride

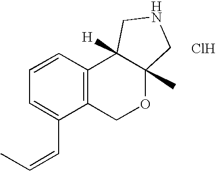

Similar procedures described in Example 137 were employed, using cis-tert-butyl 3a-methyl-6-((Z)-prop-1-enyl)-1,3,3a,9b-tetrahydroisochromeno[3,4-c]pyrrole-2(5H)-carboxylate to afford cis-3a-methyl-6-((Z)-prop-1-enyl)-1,2,3,3a,5,9b-hexahydroisochromeno[3,4-c]pyrrole hydrochloride, EI-MS: m/z=230.4 [M+H]⁺.

### EXAMPLE 140

### cis-6-Chloro-3a,5-dimethyl-1,2,3,3a,5,9b-hexahydroisochromeno[3,4-c]pyrrole

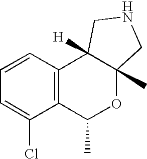

### 140.1 Preparation of cis-ethyl 6-chloro-3a-methyl-1,3,3a,9b-tetrahydroisochromeno[3,4-c]pyrrole-2(5H)-carboxylate

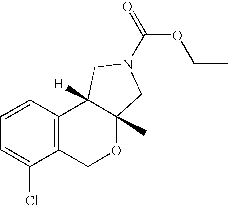

Sodium bicarbonate (40.2 mmol, 3.38 g) and ethyl chloroformate (9.66 mmol, 0.923 ml, 1.048 g) were added to a solution of cis-6-chloro-3a-methyl-1,2,3,3a,5,9b-hexahydroisochromeno[3,4-c]pyrrole (8.05 mmol, 1.8 g) in THF (20 ml) and water (20 ml). The mixture was stirred at room temperature for 16 hours and then quenched by the addition of aqueous HCl solution (1 M). The product was extracted with ethyl acetate (3×20 mL) and the combined extracts dried (MgSO₄) and concentrated under reduced pressure. The crude residue was purified by silica column chromatography (eluting with ethyl acetate in heptane, 10%) to afford cis-ethyl 6-chloro-3a-methyl-1,3,3a,9b-tetrahydroisochromeno[3,4-c]pyrrole-2(5H)-carboxylate (100% yield).

### 140.2 Preparation of cis-ethyl 6-chloro-3a-methyl-5-oxo-1,3,3a,9b-tetrahydroisochromeno[3,4-c]pyrrole-2(5H)-carboxylate

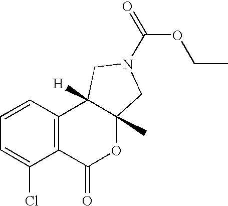

To a suspension of cis-ethyl 6-chloro-3a-methyl-1,3,3a,9b-tetrahydroisochromeno[3,4-c]pyrrole-2(5H)-carboxylate (3.13 mmol, 0.927 g) was added Jones chromic acid reagent (3.13 mmol, 2.85 ml, 0.370 g). The mixture was stirred for 2 h then water added and the mixture was extracted with DCM (×2). The combined organic extracts were washed with saturated aqueous NaHCO₃, dried (Na₂SO₄) and concentrated to afford a residue. Silica column chromatography of the residue (eluting with ethyl acetate in heptane, 10 to 50%) afforded cis-ethyl 6-chloro-3a-methyl-5-oxo-1,3,3a,9b-tetrahydroisochromeno[3,4-c]pyrrole-2(5H)-carboxylate (62% yield).

### 140.3 Preparation of cis-ethyl 6-chloro-3a,5-dimethyl-1,3,3a,9b-tetrahydroisochromeno[3,4-c]pyrrole-2(5H)-carboxylate

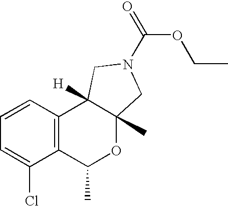

To a solution of cis-ethyl 6-chloro-3a-methyl-5-oxo-1,3,3a,9b-tetrahydroisochromeno[3,4-c]pyrrole-2(5H)-carboxylate (0.743 mmol, 0.230 g) in THF (3.71 ml) was added methyllithium (0.817 mmol, 0.510 ml). The mixture was stirred for 2 h at −78° C. then acetic acid (0.817 mmol, 0.046 ml) was added and the mixture was poured into ice-water. The aqueous layer was extracted with DCM. The combined organic extracts were dried (Na₂SO₄) then concentrated in vacuo to give crude hemiketal. The crude lactol was dissolved in DCM (25 ml), cooled to −78° C. then 2,2,2-trifluoroacetic acid (2.228 mmol, 0.165 ml, 0.254 g) was added and the mixture was stirred for 15 min. Triethylsilane (2.228 mmol, 0.360 ml, 0.259 g) was added and the mixture was stirred at −78° C. for 30 min, then allowed to warm to room temperature over 2 h. The solution was poured into ice/water and extracted with DCM. The organic extracts were dried (Na₂SO₄) and concentrated under reduced pressure. Silica column chromatography (eluting with ethyl acetate in heptane, 10 to 60%) afforded cis-ethyl 6-chloro-3a,5-dimethyl-1,3,3a,9b-tetrahydroisochromeno[3,4-c]pyrrole-2(5H)-carboxylate (70% yield), EI-MS: m/z=310.2 [M+H]⁺.

### 140.4 Preparation of cis-6-chloro-3a,5-dimethyl-1,2,3,3a,5,9b-hexahydroisochromeno[3,4-c]pyrrole

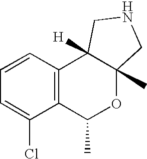

A solution of potassium hydroxide (4.84 mmol, 272 mg) in water (7595 μl) was added to a solution of cis-ethyl 6-chloro-3a,5-dimethyl-1,3,3a,9b-tetrahydroisochromeno[3,4-c]pyrrole-2(5H)-carboxylate (160 mg) in methanol (1899 μl) and the resultant solution was heated by microwave irradiation at 150° C. for 30 minutes. The mixture was cooled to room temperature then diluted with water and extracted with DCM. The combined organics were dried (Na₂SO₄) and concentrated under reduced pressure. The crude product was purified by ion exchange chromatography (SCX, 0.5 g) to afford cis-6-chloro-3a,5-dimethyl-1,2,3,3a,5,9b-hexahydroisochromeno[3,4-c]pyrrole (52% yield), EI-MS: m/z=238.1 [M+H]⁺.

### EXAMPLE 141

### cis-9-Bromo-6-chloro-3a-methyl-1,2,3,3a,5,9b-hexahydroisochromeno[3,4-c]pyrrole hydrochloride

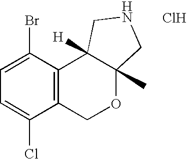

To a solution of cis-6-chloro-3a-methyl-1,2,3,3a,5,9b-hexahydroisochromeno[3,4-c]pyrrole (10.28 mmol, 2.3 g) in sulfuric acid (10.28 ml) was added N-bromosuccinimide (10.28 mmol, 1.830 g), and the mixture was stirred at room temperature for 16 h in dark. The reaction mixture was poured into ice-water (15 ml) and the mixture was washed with diethyl ether. The aqueous layer was basified with 4 N NaOH and extracted with diethyl ether. The organic extract was washed with brine, dried over Na₂SO₄ and concentrated under reduced pressure to afford cis-9-bromo-6-chloro-3a-methyl-1,2,3,3a,5,9b-hexahydroisochromeno[3,4-c]pyrrole (1.88 g, 60% yield), EI-MS: m/z=302.00, 304.00 [M+H]⁺. A sample of cis-9-bromo-6-chloro-3a-methyl-1,2,3,3a,5,9b-hexahydroisochromeno[3,4-c]pyrrole (0.066 mmol, 20 mg) was dissolved in MeOH (0.5 mL) and purified by prep-HPLC (acidic modifiers). Fractions from the peak of interest were combined, concentrated and passed through an SCX column to obtain the free base product that was readily converted to the HCl salt to afford cis-9-bromo-6-chloro-3a-methyl-1,2,3,3a,5,9b-hexahydroisochromeno[3,4-c]pyrrole hydrochloride (17 mg, 76% yield), EI-MS: m/z=302.00, 304.00 [M+H]⁺.

### EXAMPLE 142

### cis-6-Chloro-3a,9-dimethyl-1,2,3,3a,5,9b-hexahydroisochromeno[3,4-c]pyrrole hydrochloride

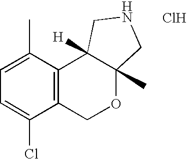

A mixture of cis-9-bromo-6-chloro-3a-methyl-1,2,3,3a,5,9b-hexahydroisochromeno[3,4-c]pyrrole (0.099 mmol, 30 mg), trimethylboroxine (0.198 mmol, 0.056 mL, 49.8 mg), tetrakis(triphenylphosphine)Pd(0) (9.91 μmol, 11.46 mg) and potassium carbonate (0.198 mmol, 27.4 mg) in degassed dioxane (2 mL) was subjected to microwave irradiation at 120° C. for 20 minutes. The reaction mixture was partitioned between EtOAc and water. The phases were separated and the aqueous was further extracted with EtOAc (2×). The combined organic extracts were dried over MgSO₄, filtered and concentrated under reduced pressure to afford the crude residue that was purified by prep-HPLC. Analysis by LCMS indicated a mixture of starting material and product—no separation on LCMS.

The sample was resubjected to the above reaction conditions to drive the reaction to completion and purified by prep-HPLC and the pure fractions were passed through an SCX cartridge and then converted to the HCl salt to afford cis-6-chloro-3a,9-dimethyl-1,2,3,3a,5,9b-hexahydroisochromeno[3,4-c]pyrrole hydrochloride (8.4 mg, 31% yield) as a white solid, EI-MS: m/z=238.00 [M+H]⁺.

### EXAMPLE 143

### cis-6-Chloro-3a-methyl-9-vinyl-1,2,3,3a,5,9b-hexahydrosochromeno[3,4-c]pyrrole

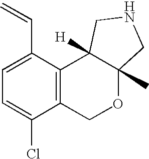

Tetrakis(triphenylphosphine)Pd(0) (8.26 μmol, 9.55 mg) was added in one portion to a mixture of cis-9-bromo-6-chloro-3a-methyl-1,2,3,3a,5,9b-hexahydroisochromeno[3,4-c]pyrrole (0.099 mmol, 50 mg), 2,4,6-trivinylcyclotroboroxane pyridine complex (0.198 mmol, 47.7 mg) and potassium carbonate (0.248 mmol, 34.3 mg) in 1,4-dioxane (2 mL) and water (0.2 mL). The mixture was subjected to microwave irradiation at 130° C. for 20 min. The reaction mixture was partitioned between DCM and water, and the organic phase was dried over Na₂SO₄ and concentrated under reduced pressure to give a crude residue that was passed through an SCX cartridge (2 g) to afford cis-6-chloro-3a-methyl-9-vinyl-1,2,3,3a,5,9b-hexahydrosochromeno[3,4-c]pyrrole (35 mg, 85%), EI-MS: m/z=250.20 [M+H]⁺.

### EXAMPLE 144

### cis-6-Chloro-9-ethyl-3a-methyl-1,2,3,3a,5,9b-hexahydroisochromeno[3,4-c]pyrrole hydrochloride

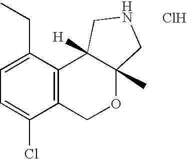

To cis-6-chloro-3a-methyl-9-vinyl-1,2,3,3a,5,9b-hexahydroisochromeno[3,4-c]pyrrole (0.140 mmol, 35.0 mg) was added ethanol (3.052 mL) and the resulting solution was degassed with nitrogen. 10% Palladium on carbon (7.01 μmol, 7.36 mg) was added and the mixture was stirred under hydrogen (balloon) for 2 h. The mixture was then filtered through Dicalite and the resulting filtrate was concentrated to afford a crude product that was purified by prep-HPLC and passed through an SCX cartridge to afford the free base product that was converted to the HCl salt affording cis-6-chloro-9-ethyl-3a-methyl-1,2,3,3a,5,9b-hexahydroisochromeno[3,4-c]pyrrole hydrochloride (15 mg, 37%), EI-MS: m/z=252.20 [M+H]⁺.

### EXAMPLE 145

### cis-6,9-Dichloro-3a-methyl-1,2,3,3a,5,9b-hexahydroisochromeno[3,4-c]pyrrole hydrochloride

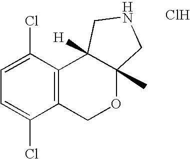

A mixture of cis-9-bromo-6-chloro-3a-methyl-1,2,3,3a,5,9b-hexahydroisochromeno[3,4-c]pyrrole (0.126 mmol, 38 mg), and nickel(II) chloride (0.502 mmol, 65.1 mg) in N-methyl-2-pyrrolidinone (2 ml) was subjected to microwave irradiation at 210° C. for 20 minutes. The reaction mixture was diluted with MeOH and passed through an SCX cartridge (2 g). The resulting filtrate was concentrated in vacuo and then dissolved in MeOH and purified by prep-HPLC. The purified fractions were concentrated in vacuo and passed through an SCX cartridge to convert to the free base product before converting to the HCl salt, affording cis-6,9-dichloro-3a-methyl-1,2,3,3a,5,9b-hexahydroisochromeno[3,4-c]pyrrole hydrochloride (17.8 mg, 48%), EI-MS: m/z=258.00 [M+H]⁺.

### EXAMPLE 146

### cis-6-Chloro-9-methoxy-3a-methyl-1,2,3,3a,5,9b-hexahydroisochromeno[3,4-c]pyrrole hydrochloride

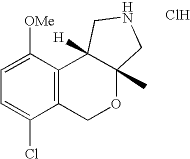

cis-9-Bromo-6-chloro-3a-methyl-1,2,3,3a,5,9b-hexahydroisochromeno[3,4-c]pyrrole (0.102 mmol, 31 mg) was transferred into a microwave vial, then treated with copper(I) bromide (0.05 mmol, 7 mg) and 25% sodium methoxide in MeOH (1 mL) and the mixture was irradiated for 30 min at 120° C. The reaction mixture was then dissolved in excess MeOH and passed through an SCX cartridge (2 g) and then purified by silica column chromatography (4 g, eluting with 5-10% MeOH in DCM) to afford the purified product that was converted to the HCl salt to afford cis-6-chloro-9-methoxy-3a-methyl-1,2,3,3a,5,9b-hexahydroisochromeno[3,4-c]pyrrole hydrochloride (6.2 mg, 21%), EI-MS: m/z=254.00 [M+H]⁺.

### EXAMPLE 147

### cis-6-(2-Fluoroethoxy)-3a-methyl-1,2,3,3a,5,9b-hexahydroisochromeno[3,4-c]pyrrole hydrochloride

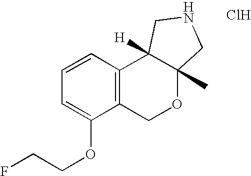

To a solution of cis-tert-butyl 6-hydroxy-3a-methyl-1,3,3a,9b-tetrahydroisochromeno[3,4-c]pyrrole-2(5H)-carboxylate (0.098 mmol, 30 mg) in DMF (3 ml) was added sodium hydride (0.196 mmol, 7.86 mg). The reaction mixture was stirred at room temperature for min then 1-fluoro-2-iodoethane (0.147 mmol, 25.6 mg) added. The mixture was stirred at room temperature for 1 h. The mixture was partitioned between water and dichloromethane. The organic extracts were washed with brine, dried over Na₂SO₄ and concentrated in vacuo. The residue was purified by prep-HPLC to afford cis-tert-butyl 6-(2-fluoroethoxy)-3a-methyl-1,3,3a,9b-tetrahydroisochromeno[3,4-c]pyrrole-2(5H)-carboxylate (28 mg, 81%). EI-MS: m/z=352.7 [M+H]⁺. 5 N HCl (0.154 ml) was added to a solution of cis-tert-butyl 6-(2-fluoroethoxy)-3a-methyl-1,3,3a,9b-tetrahydroisochromeno[3,4-c]pyrrole-2(5H)-carboxylate (0.077 mmol, 28 mg) in dioxane (2.0 ml)/MeOH (0.24 ml). The mixture was stirred at 70° C. for 1 h. The solvent was removed under reduced pressure to afford cis-6-(2-fluoroethoxy)-3a-methyl-1,2,3,3a,5,9b-hexahydroisochromeno[3,4-c]pyrrole hydrochloride (21 mg, 96%). EI-MS: m/z=252.2 [M+H]⁺.

### EXAMPLE 148

### cis-6-(Cyclopropylmethoxy)-3a-methyl-1,2,3,3a,5,9b-hexahydroisochromeno[3,4-c]pyrrole hydrochloride

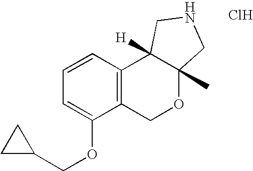

To a solution of cis-tert-butyl 6-hydroxy-3a-methyl-1,3,3a,9b-tetrahydroisochromeno[3,4-c]pyrrole-2(5H)-carboxylate (0.098 mmol, 30 mg) in DMF (3 ml) was added sodium hydride (0.196 mmol, 7.86 mg). The reaction mixture was stirred at room temperature for 10 min, then (iodomethyl)cyclopropane (0.147 mmol, 26.8 mg) added. The mixture was stirred at room temperature for 1 h. The mixture was partitioned between water and dichloromethane. The organic phase was washed with brine, dried over Na₂SO₄ and concentrated in vacuo. The residue was purified by prep-HPLC to afford cis-tert-butyl 6-(cyclopropylmethoxy)-3a-methyl-1,3,3a,9b-tetrahydroisochromeno[3,4-c]pyrrole-2(5H)-carboxylate (27.8 mg, 79%). EI-MS: m/z=360.3 [M+H]⁺. 5 N HCl (0.152 ml) was added to a solution of cis-tert-butyl 6-(cyclopropylmethoxy)-3a-methyl-1,3,3a,9b-tetrahydroisochromeno[3,4-c]pyrrole-2(5H)-carboxylate (0.076 mmol, 27.4 mg) in dioxane/MeOH. The mixture was stirred at 70° C. for 1 h. The solvent was then removed under reduced pressure to afford cis-6-(cyclopropylmethoxy)-3a-methyl-1,2,3,3a,5,9b-hexahydroisochromeno[3,4-c]pyrrole hydrochloride (19.7 mg, 87%). EI-MS: m/z=260.2 [M+H]⁺.

### EXAMPLE 149

### cis-3a-Methyl-6-(methylthio)-1,2,3,3a,5,9b-hexahydroisochromeno[3,4-c]pyrrole 2,2,2-trifluoroacetate

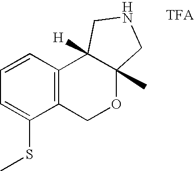

To a mixture of cis-tert-butyl 3a-methyl-6-(trifluoromethylsulfonyloxy)-1,3,3a,9b-tetrahydroisochromeno[3,4-c]pyrrole-2(5H)-carboxylate (0.046 mmol, 20 mg), BINAP (4.57 μmol, 2.85 mg) and palladium(II) acetate (4.57 μmol, 1.026 mg) in toluene (2 mL) was added sodium methanethiolate (0.091 mmol, 6.41 mg). The mixture was subjected to a microwave irradiation for 30 minutes at 120° C. Further BINAP (4.57 μmol, 2.85 mg), palladium(II) acetate (4.57 μmol, 1.026 mg) and sodium methanethiolate (0.091 mmol, 6.41 mg) were added to the mixture, and the mixture was subjected to a microwave irradiation for 60 minutes at 125° C. The mixture was partitioned between DCM and water. The aqueous layer was extracted with DCM and combined organic extracts were washed with brine, dried over Na₂SO₄ and concentrated under reduced pressure to give a residue, which was purified with prep-HPLC to afford cis-tert-butyl 3a-methyl-6-(methylthio)-1,3,3a,9b-tetrahydroisochromeno[3,4-c]pyrrole-2(5H)-carboxylate (7.9 mg, 51%). EI-MS: m/z=336.7 [M+H]⁺. 5 N HCl (0.047 ml) was added to a solution of cis-tert-butyl 3a-methyl-6-(methylthio)-1,3,3a,9b-tetrahydroisochromeno[3,4-c]pyrrole-2(5H)-carboxylate (0.024 mmol, 7.9 mg) in dioxane/MeOH. The mixture was stirred at 70° C. for 1 h. The solvent was removed and the resulting residue was purified by prep-HPLC (TFA modifier) to afford cis-3a-methyl-6-(methylthio)-1,2,3,3a,5,9b-hexahydroisochromeno[3,4-c]pyrrole 2,2,2-trifluoroacetate (4.3 mg, 52%). EI-MS: m/z=236.3 [M+H]⁺.

### EXAMPLE 150

### cis-N,N,3a-Trimethyl-1,2,3,3a,5,9b-hexahydroisochromeno[3,4-c]pyrrol-6-amine dihydrochloride

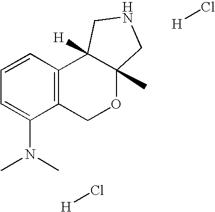

### 150.1 Preparation of cis-tert-butyl 6-bromo-3a-methyl-1,3,3a,9b-tetrahydroisochromeno[3,4-c]pyrrole-2(5H)-carboxylate

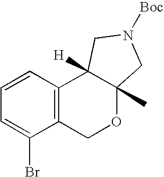

cis-6-Bromo-3a-methyl-1,2,3,3a,5,9b-hexahydroisochromeno[3,4-c]pyrrole (21.03 mmol, 5.64 g) was dissolved in DCM (200 ml). Triethylamine (42.1 mmol, 5.85 ml, 4.26 g) and di-tert-butyl dicarbonate (31.5 mmol, 6.89 g) were added and the reaction mixture was stirred overnight and then washed with washed with 1 N HCl (2×40 ml). The organic extract was dried over Na₂SO₄ and concentrated in vacuo to afford a brown oil that was purified by silica column chromatography (eluting with EtOAc in heptane, 20-30%) to afford cis-tert-butyl 6-bromo-3a-methyl-1,3,3a,9b-tetrahydroisochromeno[3,4-c]pyrrole-2(5H)-carboxylate as a colourless oil.

### 150.2 Preparation of cis-N,N,3a-trimethyl-1,2,3,3a,5,9b-hexahydroisochromeno[3,4-c]pyrrol-6-amine dihydrochloride

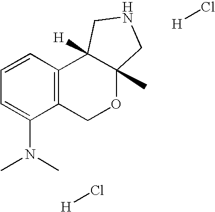

cis-tert-Butyl 6-bromo-3a-methyl-1,3,3a,9b-tetrahydroisochromeno[3,4-c]pyrrole-2(5H)-carboxylate (80 mg, 0.2 mmol), Pd₂(dba)₃ (3 mg, 3.3 μmol), (±)-BINAP (6 mg, 9.8 μmol), sodium tert-butoxide (32 mg, 0.3 mmol) and dimethylamine (49 mg, 1.1 mmol) were dissolved in toluene (5 ml) and the resulting mixture was heated in a microwave reactor at 150° C. for 10 minutes. The reaction mixture was then diluted with DCM (2 ml) and acetonitrile (1 ml) and TFA (1 ml) was added. The reaction mixture was stirred overnight and then passed through an SCX cartridge (0.5 g). The resulting filtrate was concentrated in vacuo and then purified by prep-HPLC (basic). The purified fractions were passed through an SCX cartridge (0.5 g) and the resulting filtrate was concentrated in vacuo before redissolving in 2 N HCl in MeOH and then reconcentrating in vacuo to give cis-N,N,3a-trimethyl-1,2,3,3a,5,9b-hexahydroisochromeno[3,4-c]pyrrol-6-amine dihydrochloride as a white solid, ¹H-NMR (400 MHz, MeOD) ppm 7.72 (1H, d, Ar—H), 7.55-7.50 (1H, m, Ar—H), 7.42 (1H, m, Ar—H), 5.23 (1H, d, CHHO), 5.05 (1H, d, CHHO), 3.92-3.85 (1H, m, CHHN), 3.53-3.28 (10H) 1.45 (3H, s, CH₃).

### EXAMPLE 151

### cis-N-Ethyl-N,3a-dimethyl-1,2,3,3a,5,9b-hexahydroisochromeno[3,4-c]pyrrol-6-amine dihydrochloride

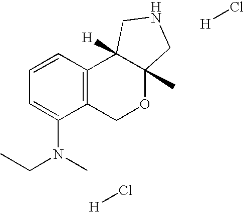

cis-tert-Butyl 6-bromo-3a-methyl-1,3,3a,9b-tetrahydroisochromeno[3,4-c]pyrrole-2(5H)-carboxylate (80 mg, 0.2 mmol), Pd₂(dba)₃ (3 mg, 3.3 μmol), (±)-BINAP (6 mg, 9.8 μmol), sodium tert-butoxide (32 mg, 0.3 mmol) and N-methylethanamine (64 mg, 1.1 mmol) were dissolved in toluene (5 ml) and the resulting mixture was heated in a microwave reactor at 150° C. for 10 minutes. The reaction mixture was then diluted with DCM (2 ml) and acetonitrile (1 ml) and TFA (1 ml) was added. The reaction mixture was stirred overnight and then passed through an SCX cartridge (0.5 g). The resulting filtrate was concentrated in vacuo and then purified by prep-HPLC (basic). The purified fractions were then passed through an SCX cartridge (0.5 g) and the resulting filtrate was concentrated in vacuo before redissolving in 2 N HCl in MeOH and then reconcentrating in vacuo to give cis-N-ethyl-N,3a-dimethyl-1,2,3,3a,5,9b-hexahydroisochromeno[3,4-c]pyrrol-6-amine dihydrochloride as a white solid, EI-MS: m/z=247.4 [M+H]⁺.

### EXAMPLE 152

### cis-N-Isopropyl-N,3a-dimethyl-1,2,3,3a,5,9b-hexahydroisochromeno[3,4-c]pyrrol-6-amine dihydrochloride

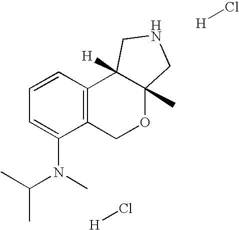

cis-tert-Butyl 6-bromo-3a-methyl-1,3,3a,9b-tetrahydroisochromeno[3,4-c]pyrrole-2(5H)-carboxylate (80 mg, 0.2 mmol), Pd₂(dba)₃ (3 mg, 3.3 μmol), (±)-BINAP (6 mg, 9.8 μmol), sodium tert-butoxide (32 mg, 0.3 mmol) and N-methylpropan-2-amine (64 mg, 1.1 mmol) were dissolved in toluene (5 ml) and the resulting mixture was heated in a microwave reactor at 150° C. for 10 minutes. The reaction mixture was then diluted with DCM (2 ml) and acetonitrile (1 ml) and TFA (1 ml) was added. The reaction mixture was stirred overnight and then passed through an SCX cartridge (0.5 g). The resulting filtrate was concentrated in vacuo and then purified by prep-HPLC (basic). The purified fractions were passed through an SCX cartridge (0.5 g) and the resulting filtrate was concentrated in vacuo before redissolving in 2 N HCl in MeOH and then reconcentrating in vacuo to give cis-N-isopropyl-N,3a-dimethyl-1,2,3,3a,5,9b-hexahydroisochromeno[3,4-c]pyrrol-6-amine dihydrochloride as a white solid, EI-MS: m/z=261.2 [M+H]⁺.

### EXAMPLE 153

### trans-7-Bromo-6-chloro-2,3a-dimethyl-1,2,3,3a,5,9b-hexahydroisochromeno[3,4-c]pyrrole

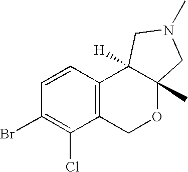

trans-7-Bromo-6-chloro-3a-methyl-1,2,3,3a,5,9b-hexahydroisochromeno[3,4-c]pyrrole (20 mg) was dissolved in DCM (5 mL). Formaldehyde (5.05 mmol, 140 μl, 152 mg), 5 drops of glacial acetic acid and sodium triacetoxyborohydride (0.193 mmol, 41 mg) was added and the reaction mixture was stirred at room temperature for 1 h before diluting with 5% aq. Na₂CO₃. The phases were separated and the organic extract was dried over Na₂SO₄ and concentrated in vacuo to give a crude oil that was purified by silica column chromatography (2 g silica, eluting with 2-10% MeOH in DCM) to afford trans-7-bromo-6-chloro-2,3a-dimethyl-1,2,3,3a,5,9b-hexahydroisochromeno[3,4-c]pyrrole (7 mg, 33%), EI-MS: m/z=318.0 [M+H]⁺.

### EXAMPLE 154

### cis-2-Benzyl-6-chloro-3a-methyl-7-(prop-1-en-2-yl)-1,2,3,3a,5,9b-hexahydroisochromeno[3,4-c]pyrrole

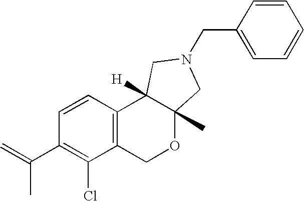

Tetrakis(triphenylphosphine)palladium(0) (8.91 μmol, 10.30 mg) was added in one portion to a mixture of cis-2-benzyl-7-bromo-6-chloro-3a-methyl-1,2,3,3a,5,9b-hexahydroisochromeno[3,4-c]pyrrole (0.178 mmol, 0.07 g), 2-isopropenyl-4,4,5,5-tetramethyl-1,3,2-dioxaborolane (0.214 mmol, 0.036 g), and potassium carbonate (0.267 mmol, 0.037 g) in a solution of degassed 1,4-dioxane (1.5 ml)/Water (0.3 ml). The mixture was subjected to microwave irradiation at 130° C. for 20 minutes. Further tetrakis(triphenylphosphine)palladium(0) (8.91 μmol, 10.30 mg), 2-isopropenyl-4,4,5,5-tetramethyl-1,3,2-dioxaborolane (0.214 mmol, 0.036 g), and potassium carbonate (0.267 mmol, 0.037 g) were added and the reaction mixture was again irradiated at 130° C. for 20 minutes. The reaction mixture was then diluted with water (10 ml) and 4 N NaOH (2 ml) and extracted with EtOAc (3×10 ml). The combined organic extracts were washed with brined, dried over Na₂SO₄ and concentrated in vacuo to afford a crude oil that was purified by silica column chromatography (4 g silica, eluting with 10-60% EtOAc in heptane) to afford cis-2-benzyl-6-chloro-3a-methyl-7-(prop-1-en-2-yl)-1,2,3,3a,5,9b-hexahydro isochromeno[3,4-c]pyrrole (6 mg, 10%), m/z=354.2 [M+H]⁺.

### EXAMPLE 155

### cis-2-Benzyl-6-chloro-3a,7-dimethyl-1,2,3,3a,5,9b-hexahydroisochromeno[3,4-c]pyrrole

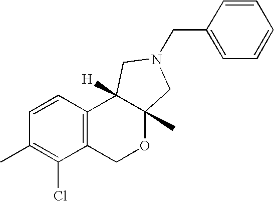

A solution of cis-2-benzyl-7-bromo-6-chloro-3a-methyl-1,2,3,3a,5,9b-hexahydroisochromeno[3,4-c]pyrrole (0.178 mmol, 0.07 g), tetrakis(triphenylphosphine)Pd(0) (8.91 μmol, 10.40 mg), potassium carbonate (0.267 mmol, 0.037 g) and trimethylboroxine (0.214 mmol, 0.060 ml, 0.054 g) in 1,4-dioxane (1.5 ml) and water (0.3 ml) was heated in a microwave reactor at 130° C. for 30 min. Further tetrakis(triphenylphosphine)Pd(0) (8.91 μmol, 10.40 mg), potassium carbonate (0.267 mmol, 0.037 g) and trimethylboroxine (0.214 mmol, 0.060 ml, 0.054 g) was added and the reaction was again irradiated for 30 min at 130° C. Further tetrakis(triphenylphosphine)Pd(0) (8.91 μmol, 10.40 mg), potassium carbonate (0.267 mmol, 0.037 g) and trimethylboroxine (0.214 mmol, 0.060 ml, 0.054 g) was added and the reaction was again irradiated for 30 min at 130° C. The solvent was then removed under reduced pressure and the resulting residue was diluted with 5% aq Na₂CO₃ soln. (10 ml) and extracted with EtOAc (2×10 ml). The combined organic extracts were dried over Na₂SO₄, filtered through Dicalite and concentrated in vacuo to give a crude oil that was purified by silica column chromatography (4 g silica, 10-60% EtOAc in heptane) to afford cis-2-benzyl-6-chloro-3a,7-dimethyl-1,2,3,3a,5,9b-hexahydroisochromeno[3,4-c]pyrrole (50 mg, 86%), m/z=328.3 [M+H]⁺.

### EXAMPLE 156

### cis-6-Chloro-3a,7-dimethyl-1,2,3,3a,5,9b-hexahydroisochromeno[3,4-c]pyrrole hydrochloride

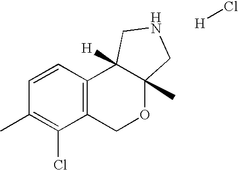

A solution of cis-2-benzyl-6-chloro-3a,7-dimethyl-1,2,3,3a,5,9b-hexahydroisochromeno[3,4-c]pyrrole (0.153 mmol, 0.05 g) in dry toluene (1.5 ml) was treated with 1-chloroethyl chloroformate (0.763 mmol, 0.082 ml, 0.109 g) and subjected to microwave irradiation at 160° C. for 20 minutes. Methanol (0.4 ml) was then added and the mixture was again subjected to microwave irradiation at 160° C. for 5 minutes. The reaction mixture was concentrated in vacuo and the resulting residue was passed through an SCX cartridge (0.5 g) then purified by silica column chromatography (2 g, silica, eluting with 2-25% MeOH in DCM). The purified fractions were concentrated in vacuo, then dissolved in 2 N HCl in MeOH and concentrated in vacuo to give cis-6-chloro-3a,7-dimethyl-1,2,3,3a,5,9b-hexahydroisochromeno[3,4-c]pyrrole hydrochloride (7 mg, 17%), m/z=238.0 [M+H]⁺.

### EXAMPLE 157

### Chiral Resolution

Selected compounds were chirally resolved by supercritical fluid chromatography (SFC): The majority of compounds run on a Chiralpak ADH column 25 cm×0.46 cm by SFC using conditions—4 ml/min, 220 nm, 35° C., 100 bar CO₂. A few were resolved using Chiralcel OJH column or Chiralpak ASH column. A summary of the column and conditions for each compound is shown in Table 1 below.

TABLE 1
Column and eluent for SFC chiral resolutions of selected compounds

| | | | | | Run |
|---|---|---|---|---|---|
| | | | % | | time |
| Compound | Column | Eluent | Composition | Resolution | (min) |
| 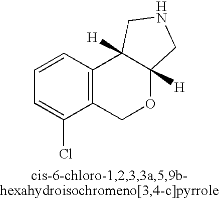 | ASH | Propanol/0.2% DEA | 20 | 0.9 | 3.5 |
| 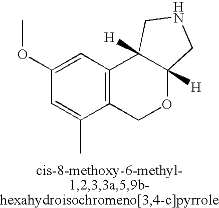 | ADH | Ethanol/0.1% DEA | 30 | 1.01 | 4 |
| 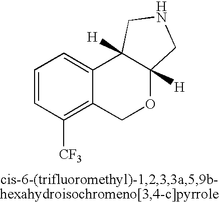 | ADH | Methanol/0.1% DEA | 30 | 2.92 | 3.5 |
| 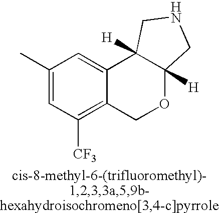 | ADH | Ethanol/0.1% ADH | 20 | 1.44 | 3 |
| 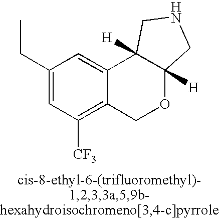 | ADH | Propanol/0.2% IPam | 20 | 1.49 | 3.5 |
| 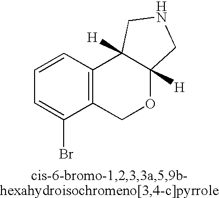 | ADH | Propanol/0.2% IPam | 40 | 1.95 | 3.5 |
| 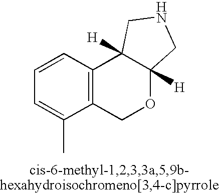 | ADH | Propanol/0.2% IPam | 30 | 2.86 | 4 |
| 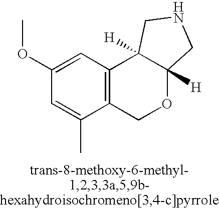 | ADH | Propanol/0.2% IPam | 25 | 0.93 | 4 |
| 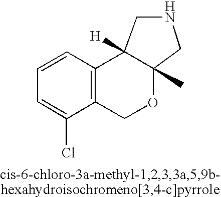 | ADH | Propanol/0.2% IPam | 25 | 1 | 4 |
| 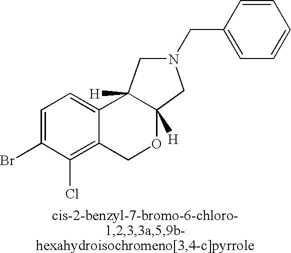 | OJH | Propanol/0.2% IPam | 30 | 1.52 | 5 |
| 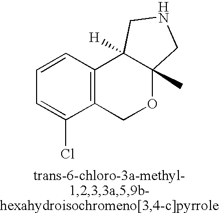 | ADH | Methanol/0.1% DEA | 25 | 1.01 | 4.5 |
| 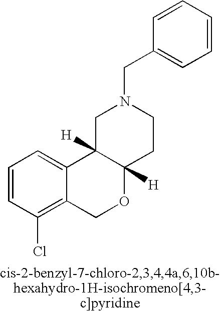 | ADH | Prpanol/0.2% IPam | 15 | 0.85 | 4 |
| 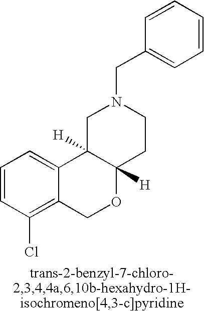 | ADH | Ethanol/0.1% DEA | 25 | 0.94 | 3.5 |
| 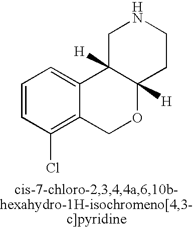 | ADH | Ethanol/0.1% DEA | 30 | 1.25 | 4 |
| 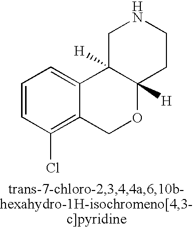 | ADH | Ethanol/0.1% DEA | 40 | 3.31 | 5 |
| 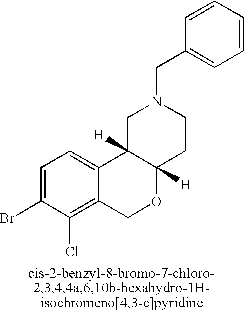 | OJH | Ethanol/0.1% DEA | 20 | 0.9 | 6 |
| 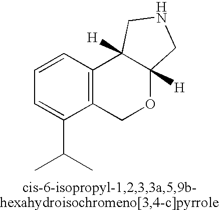 | ADH | Methanol/0.1% DEA | 30 | 2.77 | 4.5 |
| 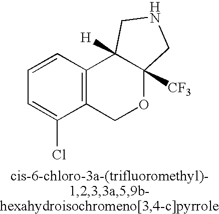 | ADH | Methanol/0.1% DEA | 25 | 2.4 | 4 |
| 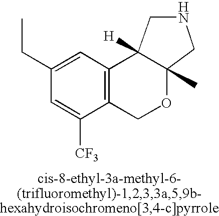 | ADH | Propanol/0.2% IPam | 10 | 1.02 | 6 |
| 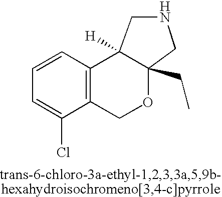 | ADH | Methanol/0.1% DEA | 20 | 1.13 | 4 |
| 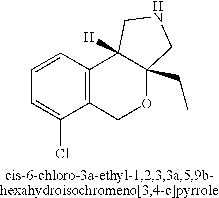 | ADH | Propanol/0.2% IPam | 25 | 0.91 | 4 |
| 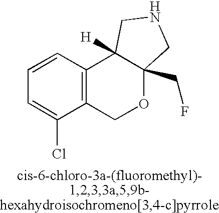 | ADH | Propanol/0.2% IPam | 25 | 1.07 | 4 |
| 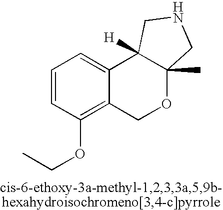 | ADH | Propanol/0.2% IPam | 25 | 0.95 | 3.5 |
| 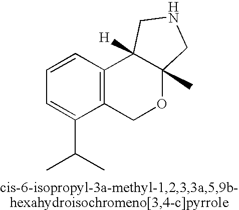 | ADH | Propanol/0.2% IPam | 30 | 1.12 | 2 |
| 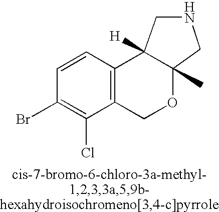 | ADH | Methanol/0.1% DEA | 35 | 1.86 | 4.5 |
| 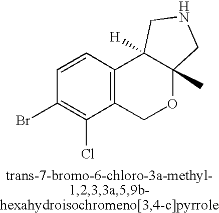 | ADH | Methanol/0.1% DEA | 35 | 2.1 | 4.5 |
| 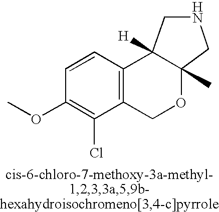 | ADH | Methanol/0.1% DEA | 25 | 1.69 | 4 |
| 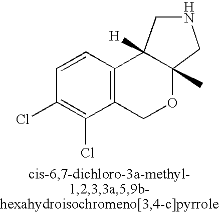 | ADH | Methanol/0.1% DEA | 30 | 1.7 | 4.5 |
| 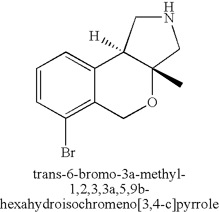 | ADH | Methanol/0.1% DEA | 40 | 1.74 | 3.5 |
| 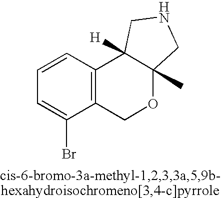 | ADH | Methanol/0.1% DEA | 35 | 1.42 | 3.5 |
| 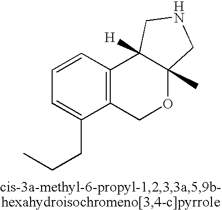 | ADH | Ethanol/0.1% DEA | 40 | 2 | 2.5 |
| 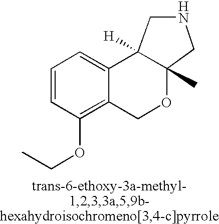 | ADH | Methanol/0.1% DEA | 25 | 2.95 | 4.3 |

### EXAMPLE 158

### In Vitro Functional Assay

The aim of this assay is to identify compounds that act as agonists at the human 5HT_{2C} (VSV) receptor, stably expressed in CHO cells, using the Fluorescent Imaging Plate Reader (FLIPR; Molecular Devices).

All handling of genetically modified CHO cells are carried out under Class II containment following the GM (contained use) regulations 2000.

Cells maintained in UltraCHO Medium (Biowhittaker), supplemented with 1% dialysed fetal bovine serum (Hyclone) and 0.4 mg/ml Geneticin (GIBCO), at 37° C. with 5% CO₂ in air, and 90% humidity. Cells are split between 2-4 days growth. Passage conditions optimised to ensure that the cell density does not exceed 90% confluence. For all experiments cells are seeded at a density of 6×10⁵/ml in plating medium (UltraCHO with 1% dialysed fetal bovine serum) then incubated at 37° C. with 5% CO2 in air, and 90% humidity for 20-24 hours prior to the assay.

Media aspirated from cells and washed once with wash buffer (1×D-PBS —CaCl₂—MgCl₂) prior to incubation with Calcium-3 dye solution (containing 2.5 mM probenecid) for 1 hour at room temperature. Compounds added from drug plate to cell plate on FLIPR prior to fluorescence intensity reading.

Data analysed using in-house programme. Increase in measured relative fluorescense units (RFU) by test compound expressed as percentage maximal response of cells (evoked by 10 μM 5HT). Concentration response curves constructed and analysed with appropriate non-linear regression 4 parameter logistic equation: y=A+((B−A)/(1+((C/x)̂D)));

where A=min Y, B=max Y, C=EC₅₀ and D=slope factor.

Exemplified compounds were found to have a pEC₅₀>6.0.

### EXAMPLE 159

### In-Vitro Radioligand Binding Assays

### 159.1 Saturation Binding Assays

Membrane homogenates from NIH-3T3 cells expressing human 5HT2C (INI) receptors are prepared prior to saturation and competition binding experiments.

Using 96 deep well plates the following is added: 100 μl DMSO for total binding (1% final concentration), 100 μl mianserin for non-specific binding (NSB, 1 μM final concentation) and 100 μl appropriate radioligand concentration. Following 1.5 hour incubation at room temperature, reaction is terminated by vacuum filtration through a cell harvester onto a pre-soaked (0.03% PEI in assay buffer (Tris HCl, pH 7.4)) Whatman GF/B filter plate. Counts per minute (cpm) determined by scintillation counter. Protein concentation of membrane determined from standard curve of known concentration of bovine serum albumin (BSA); optical density read at 595 nm. Linear regression fitted to standard curve and calculation of membrane sample protein concentation performed using GraphPad Prism 4.0 or equivalent.

### 1.2. Data Analysis

Using PRISM 4.0 or equivalent, free ligand concentration (nM) is plotted against the total, non-specific and specific binding. Non-linear regression and one site binding (hyperbola) used for calculation of ligand concentration, K_{D} (nM) and Bmax (pmol/mg protein) values:

$$y = \frac{{B}_{max} . x}{( {K}_{D} + x )}$$

### 159.2 Competition Binding Assays

The aim of these assays are to determine binding efficiency of a compound using inhibition of [3H] mesulergine (Amersham) binding to human 5HT2C (INI) receptors expressed in NIH-3T3 cells as membrane homogenates.

Clozapine used as a reference; total binding determined by 1% DMSO; and non-specific binding determined by 10 μM clozapine. Assay format uses 96 deep-well microtitre plates in a total volume of 500 μl, such that each well contains 395 μl membrane, 5 μl test compound concentation or DMSO or clozapine, and 100 μl of appropriate concentration of radioligand. Following 1.5 hour incubation at room temperature, assay terminated by vacuum filtration through a cell harvester onto pre-soaked (0.03% PEI in assay buffer) Whatman GF/B filter plates. Radiation (cpm) counted using scintillation counter.

### 159.3 Data Analysis

Results are expressed relative to the maximal clozapine binding. Percentage effect is calculated for each well by correlating the cpm value with the mean of the values of the MIN wells (0%) and with the mean of the values of the MAX wells (100%) obtained from the same plate with the following formula:

$$\% Effect = \frac{( value - MIN )}{( MAX - MIN )} \times 100 \%$$

The individual effects at each concentration are used to fit the following four-parameter curve:

$$y = A + \frac{( B - A )}{( 1 + {( \frac{{10}^{C}}{x} )}^{D} )}$$

Where A=min, B=max, C=inflection point (log₁₀ (EC₅₀)=−pEC₅₀) and D=hill slope.

Calculation of pKi, negative logarithm of the equilibrium dissociation constant, Ki

$$Ki = \frac{EC 50}{( 1 + ( [ L ] / {K}_{D} ) )}$$

Where EC₅₀=Concentration at point of inflection, [L]=radioligand concentration and K_{D}=equilibrium dissociation constant for the radioligand (expressed in the appropriate units of concentration).

### EXAMPLE 160

### Penile Erection/Head Shake Protocol

### 160.1 Introduction

Administration of 5-HT_{2C} agonists induces penile erections in rats. This phenomenon is known to be mediated by 5-HT_{2C} receptors since it can be reversed by treatment with a selective 5-HT_{2C} antagonist. Activation of the 5-HT_{2A} receptor induces head shakes, and this effect can be reversed by selective 5-HT_{2A} antagonists. The test is used to evaluate a test compound for its activity at 5-HT_{2C} and/or 5-HT_{2A} receptors (Berendsen HHG, Jenck F, Broekkamp CLE. Psychopharmacology 1990; 101: 57-61).

### 160.2 Materials and Methods

Group housed male Wistar rats (Harlan Olac Ltd., Bicester, UK) weighing 200 g+ are housed in standard conditions with food and water ad-lib.

The test is carried out in a transparent perspex observation chamber (W: 10 cm, D: 10 cm, H20 cm). The test is videoed, 2 cameras are placed in front of the chambers and 2 below the chambers enabling all round observation of the rats.

Each experiment consists of a control group and n (usually 3) groups receiving test compound.

### 160.3 Procedure

Animals are habituated to the observation chambers on at least 3 occasions prior to the experiment.

On the day of the experiment each rat is weighed and identified (usually by tail marking). The test compound or vehicle is administered. Following the pre-treatment time the rats are placed individually into the observation chambers and video recording commences.

PE and HS are usually recorded for 30 minutes.

PE and HS are considered to have occurred when the following behaviours are observed:

PE—An upright sitting position with repeated pelvic thrusts and an erect, engorged penis which the rat grooms.

HS—Sudden shaking of the head or whole body.

### 160.4 Evaluation of Responses

The mean number of PE and HS is calculated for each experimental group and statistical analysis is carried out using a one-way ANOVA followed by a Dunnetts test.

1-16. (canceled)

17. A tricyclic heterocyclic derivative of Formula I 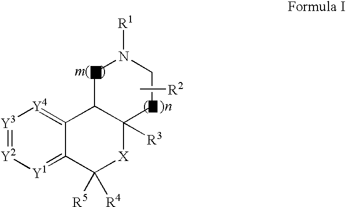
wherein,
m is 1 or 2;
n is 0 or 1;
R¹ is H, C₁₋₆alkyl, C₂₋₆alkenyl, C₂₋₆alkynyl, C₃₋₇cycloalkyl, C₃₋₇cycloalkylC₁₋₂alkyl, C₁₋₄alkyloxyC₂₋₃alkyl or C₆₋₁₀arylC₁₋₂alkyl, said C₁₋₆alkyl, C₂₋₆alkenyl, C₂₋₆alkynyl, C₃₋₇cycloalkyl, C₃₋₇cycloalkylC₁₋₂alkyl, C₁₋₄alkyloxyC₂₋₃alkyl and C₆₋₁₀arylC₁₋₂alkyl being optionally substituted with one or more halogens;
R² is H, C₁₋₆alkyl, C₃₋₇cycloalkyl or C₃₋₇cycloalkylC₁₋₂alkyl, said C₁₋₆alkyl, C₃₋₇cycloalkyl and C₃₋₇cycloalkylC₁₋₂alkyl being optionally substituted with one or more halogens;
R³ is H, C₁₋₆alkyl, C₃₋₇cycloalkyl, C₃₋₇cycloalkylC₁₋₂alkyl or C₁₋₄alkyloxyC₁₋₂alkyl, said C₁₋₆alkyl, C₃₋₇cycloalkyl, C₃₋₇cycloalkylC₁₋₂alkyl and C₁₋₄alkyloxyC₁₋₂alkyl being optionally substituted with one or more halogens;
R⁴ and R⁵ are each independently H, C₁₋₆alkyl, C₃₋₇cycloalkyl, C₃₋₇cycloalkylC₁₋₂alkyl or C₁₋₄alkyloxyC₁₋₂alkyl said C₁₋₆alkyl, C₃₋₇cycloalkyl, C₃₋₇cycloalkylC₁₋₂alkyl and C₁₋₄alkyloxyC₁₋₂alkyl being optionally independently substituted with one or more halogens or R⁴ and R⁵ together with the carbon to which they are bonded form a 3-6 membered carbocyclic ring optionally comprising a further heteroatom selected from O and S;
X is O, S, SO, SO₂, OCR^{4′}R^{5′} or CR^{4′}R^{5′}O;
R^{4′} and R^{5′} are each independently H, C₁₋₆alkyl, C₃₋₇cycloalkyl or C₃₋₇cycloalkylC₁₋₂alkyl, said C₁₋₆alkyl, C₃₋₇cycloalkyl and C₃₋₇cycloalkylC₁₋₂alkyl being optionally independently substituted with one or more halogens;
Y¹ is N or CR⁶;
Y² is N or CR⁷;
Y³ is N or CR⁸;
Y⁴ is N or CR⁹ with the proviso that no more than one of Y¹-Y⁴ can be N simultaneously;
R⁶, R⁷ and R⁸ are each independently selected from H, C₁₋₆alkyl, C₂₋₆alkenyl, C₂₋₆alkynyl, C₃₋₇cycloalkyl, C₃₋₇cycloalkylC₁₋₂alkyloxy, C₁₋₆alkyloxy, C₁₋₄alkyloxyC₁₋₂alkyl, C₁₋₆alkylSC₁₋₂alkyl, C₁₋₆alkylSO₂C₁₋₂alkyl, SC₁₋₆alkyl, SOC₁₋₆alkyl, SO₂C₁₋₆alkyl, CO₂R¹², NR¹³SO₂R¹⁴, CONR¹⁵R¹⁶, SO₂NR¹⁷R¹⁸, C₆₋₁₀aryl, C₆₋₁₀arylC₁₋₂alkyloxy, CN, halogen and a 5-6 membered saturated or unsaturated heterocyclic ring system comprising 1-2 heteroatoms independently selected from N, O and S, wherein said C₁₋₆alkyl, C₃₋₇cycloalkyl, C₃₋₇cycloalkylC₁₋₂alkyloxy and C₁₋₆alkyloxy are optionally independently substituted with one or more halogens and wherein said C₆₋₁₀aryl, C₆₋₁₀arylC₁₋₂alkyloxy and 5-6 membered saturated or unsaturated heterocyclic ring system comprising 1-2 heteroatoms independently selected from N, O and S are optionally independently substituted with one or more substituents selected from methyl, halogen and methoxy or
R⁶ and R⁷ or R⁷ and R⁸ together with the atoms to which they are bonded form a 5-7 membered unsaturated carbocyclic ring optionally comprising 1-2 heteroatoms selected from N, O and S and optionally substituted with methyl or halogen;
R⁹ is H, C₁₋₆alkyl, C₁₋₆alkyloxy, C₃₋₇cycloalkyl, CN or halogen said C₁₋₆alkyl, C₁₋₆alkyloxy and C₃₋₇cycloalkyl being optionally independently substituted with one or more halogens;
R¹⁰ and R¹¹ are each independently H, C₁₋₆alkyl, C₃₋₇cycloalkyl or COC₁₋₆alkyl said C₁₋₆alkyl being optionally substituted with one or more halogens;
R¹² is C₁₋₆alkyl;
R¹³ is H or C₁₋₆alkyl;
R¹⁴ is C₁₋₆alkyl;
R¹⁵ and R¹⁶ are each independently H or C₁₋₆alkyl and
R¹⁷ and R¹⁸ are each independently H or C₁₋₆alkyl;
with the proviso that when R⁶ and R⁹ are H, R⁷ and R⁸ cannot independently or together be H, hydroxy, methoxy or benzyloxy,
or a pharmaceutically acceptable salt thereof.

18. The tricyclic heterocyclic derivative according to claim 17, wherein m is 1 and n is 0.

19. The tricyclic heterocyclic derivative according to claim 17, wherein R¹ is H.

20. The tricyclic heterocyclic derivative according to claim 17, wherein R² is H.

21. The tricyclic heterocyclic derivative according to claim 17, wherein R³ is H, methyl, fluoromethyl, trifluoromethyl or ethyl.

22. The tricyclic heterocyclic derivative according to claim 17, wherein R⁴ and R⁵ are each independently H or methyl.

23. The tricyclic heterocyclic derivative according to claim 17, wherein X is O.

24. The tricyclic heterocyclic derivative according to claim 17, wherein Y¹ is CR⁶, Y² is CR⁷, Y³ is CR⁸ and Y⁴ is CR⁹.

25. The tricyclic heterocyclic derivative according to claim 17, wherein R⁶ is H, chloro, bromo, methyl, trifluoromethyl, ethyl, isopropenyl, (Z)-2-propenyl, n-propyl, isopropyl, cyclopropyl, 2-methylpropyl, cyclopentyl, N-methyl-N-ethylamino, N-methyl-N-isopropylamino, methoxy, ethoxy, isopropyloxy, phenyl, methylthio or N,N-dimethylamino.

26. The tricyclic heterocyclic derivative according to claim 17, wherein R⁷ is H, methyl, trifluoromethyl, ethyl, cyclopropyl, 2-methylpropyl, methoxy, bromo or chloro.

27. The tricyclic heterocyclic derivative according to claim 17, wherein R⁸ is H, methyl, trifluoromethyl, ethyl, cyclopropyl or N,N-dimethylamino.

28. The tricyclic heterocyclic derivative according to claim 17, wherein R⁹ is H, methyl, ethyl, methoxy, bromo or chloro.

29. A tricyclic heterocyclic derivative selected from: 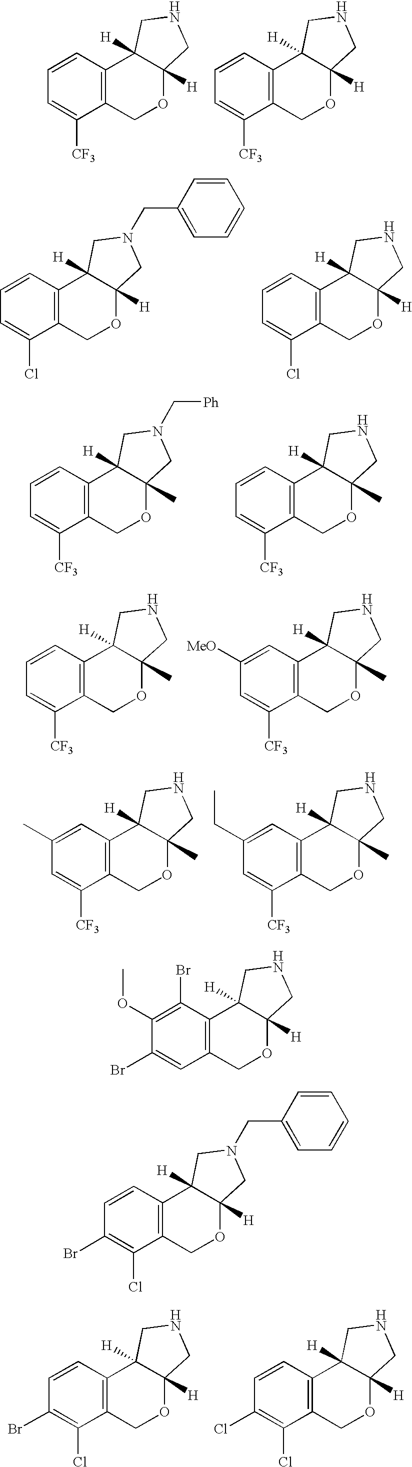 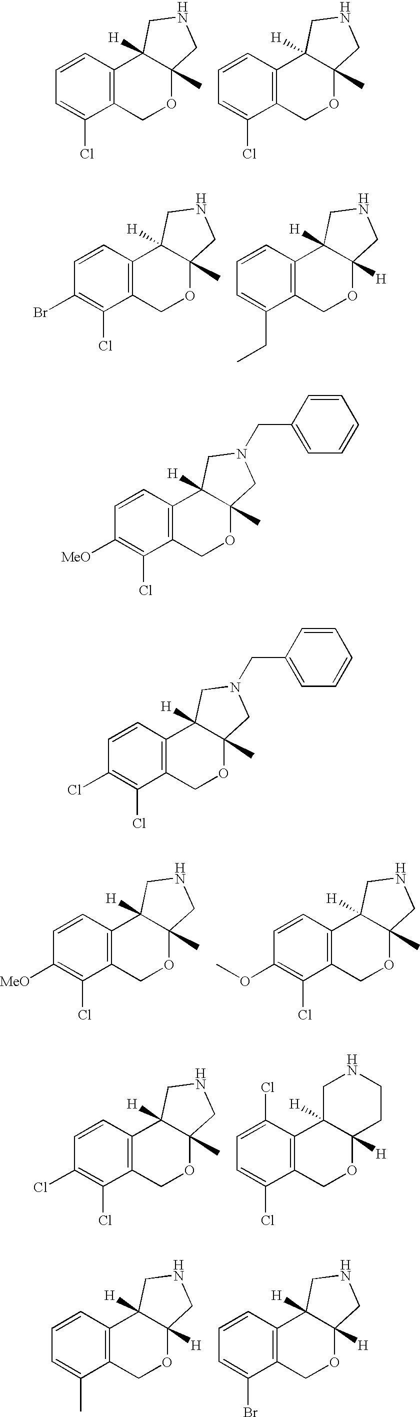 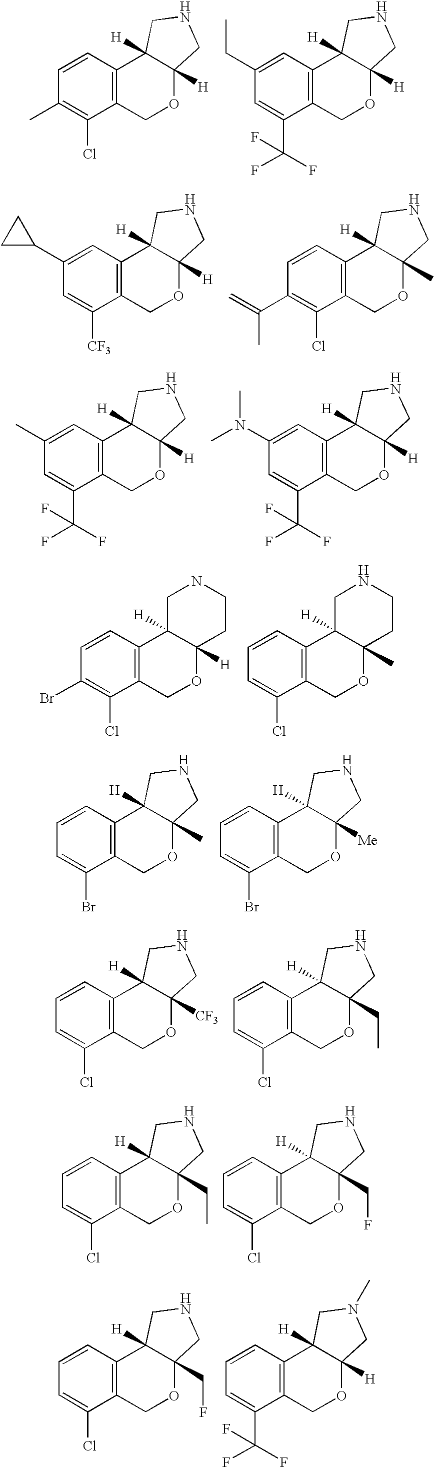 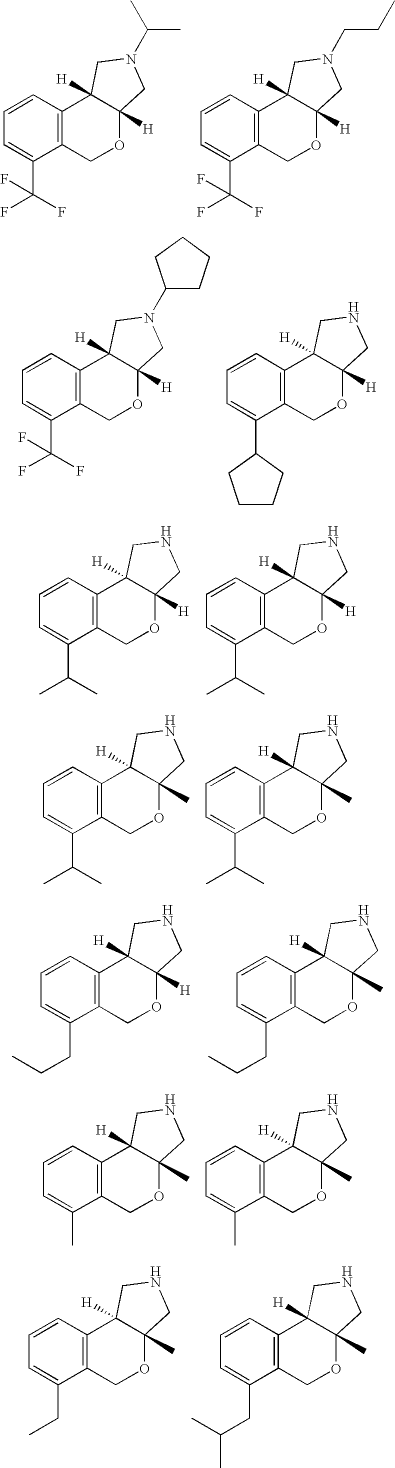 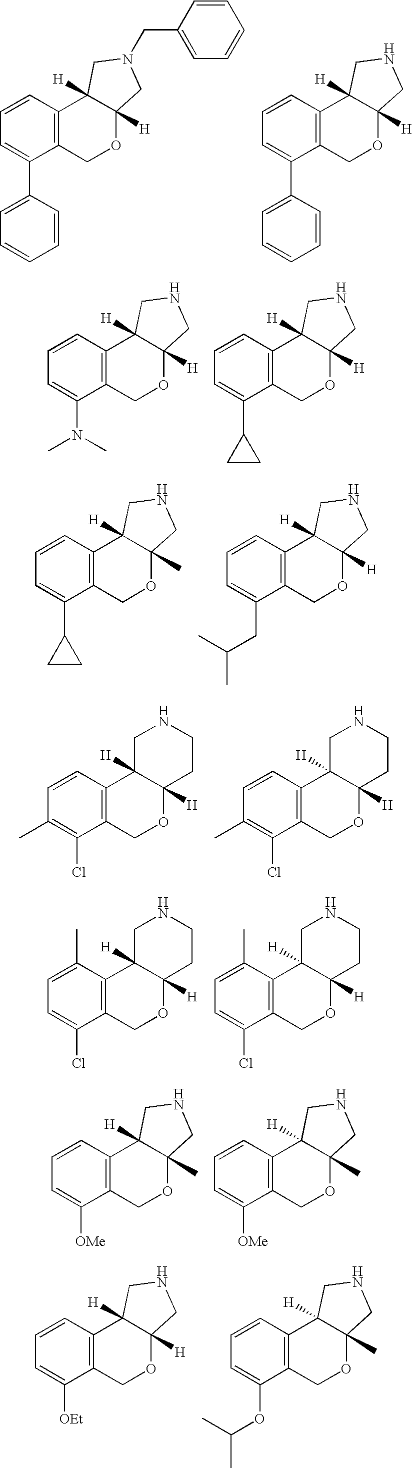 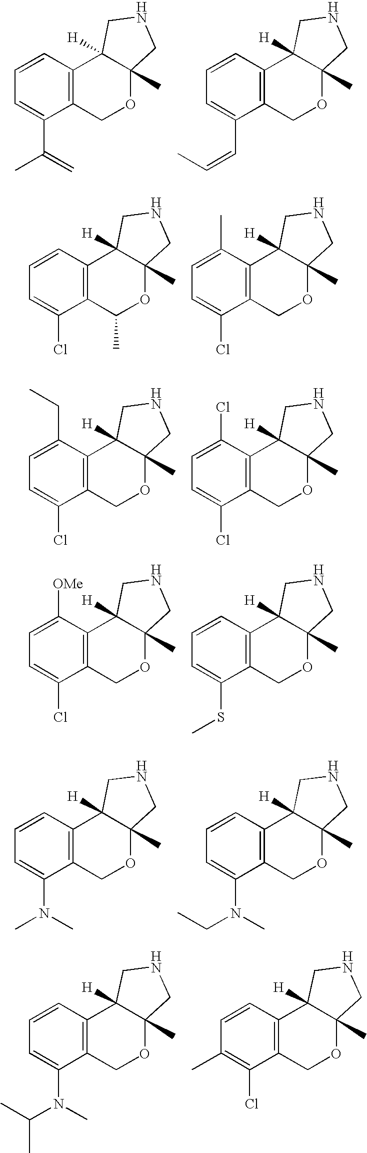
or a pharmaceutically acceptable salt thereof.

30. A pharmaceutical composition comprising a tricyclic heterocyclic derivative according to claim 17 or a pharmaceutically acceptable salt thereof in admixture with one or more pharmaceutically acceptable excipients.

31. A pharmaceutical composition comprising a tricyclic heterocyclic derivative according to claim 29 or a pharmaceutically acceptable salt thereof in admixture with one or more pharmaceutically acceptable excipients.

32. A method for the treatment in a mammal of a serotonin-mediated disorder selected from the group consisting of obesity, schizophrenia and cognitive dysfunction, the method comprising administering a therapeutically effective amount of a heterocyclic derivative according to claim 17 or a pharmaceutically acceptable salt thereof.

33. A method for the treatment in a mammal of a serotonin-mediated disorder selected from the group consisting of obesity, schizophrenia and cognitive dysfunction, the method comprising administering a therapeutically effective amount of a heterocyclic derivative according to claim 29 or a pharmaceutically acceptable salt thereof.
